(19) United States
(12) Patent Application Publication
(10) Pub. No.: US 2012/0108654 A1
(43) Pub. Date: May 3, 2012

(54) COMPOSITIONS AND METHODS FOR THE TREATMENT OF OCULAR OXIDATIVE STRESS AND RETINITIS PIGMENTOSA

(75) Inventor: Peter A. Campochiaro, Baltimore, MD (US)

(73) Assignee: The Johns Hopkins University, Baltimore, MD (US)

(21) Appl. No.: 13/002,243

(22) PCT Filed: Jun. 30, 2009
(86) PCT No.: PCT/US09/03925

§ 371 Date: Mar. 31, 2011

Related U.S. Application Data
(60) Provisional application No. 61/133,500, filed on Jun. 30, 2008.
(60) Provisional application No. 61/220,852, filed on Jun. 26, 2009.

(51) Int. Cl. A61K 31/7088; A61P 27/02; A61P 9/10; A61P 39/06; A61P 9/04; A61P 25/28; A61P 3/10; A61P 9/00; C12N 15/63; A61P 25/16

(52) U.S. Cl. 514/44 R; 435/320.1

(57) ABSTRACT

Oxidative damage contributes to cone cell death in retinitis pigmentosa and death of rods, cones, and retinal pigmented epithelial (RPE) cells in ocular oxidative stress related diseases including age-related macular degeneration and retinitis pigmentosa. Oral antioxidants may provide modest benefits, but more efficient ways of preventing oxidative damage are needed. Compositions and methods are provided herein for the prevention, amelioration, and/or treatment of early or late stage ocular disease by increasing the expression or activity of one or more peroxidases in cells of the eye, particularly retinal cells, and further optionally increasing the expression or activity of one or more superoxide dismuatases in the same cells.

### REFERENCE TO RELATED APPLICATIONS

This application is related to U.S. Provisional Patent Application Ser. Nos. 61/133,500 and 61/220,852; filed Jun. 30, 2008 and Jun. 26, 2009, respectively, and which are incorporated herein by reference in their entirety.

### GOVERNMENT SUPPORT

This work was supported by NEI Grants EY05951 and P30EY1765 from the National Institutes of Health. The Government has certain rights in this application.

### BACKGROUND

Retinal photoreceptors are packed with mitochondria and have extremely high metabolic activity and oxygen consumption. Since run-off from the electron transport chain is a major source of oxidative stress, photoreceptors are challenged under normal circumstances. In patients with retinitis pigmentosa (RP), one of a number of different mutations causes death of rods which drastically reduces oxygen consumption and elevates oxygen levels in the outer retina. Prolonged exposure to high levels of oxygen causes progressive oxidative damage to cones (Shen et al., 2005. J. Cell Physiol. 203:457-464), and their gradual death results in progressive constriction of visual fields and eventual blindness. Antioxidants significantly slow cone cell death in several models of RP; therefore, clinical trials investigating the effects of antioxidants in patients with RP are being planned.

Oxidative damage has also been implicated in another highly prevalent eye disease, age-related macular degeneration (AMD). One of the first hints came from epidemiologic studies that showed a negative correlation between the presence of AMD and consumption of a diet rich in antioxidants. This led to the Age-Related Eye Disease Study in which it was shown that antioxidant vitamins and/or zinc reduced the risk of progression to advanced AMD and severe loss of vision (Group, 2001. Arch. Ophthalmol. 119:1417-1436). The protective effects of AREDS formulation is clinically meaningful and it is now part of standard care in AMD patients with phenotypic characteristics associated with a high risk of progression; however, despite its use there are still large number of patients that develop advanced AMD.

### SUMMARY OF THE INVENTION

The invention provides compositions and methods for the prevention, amelioration, and/or treatment of ocular diseases associated with oxidative stress. The invention further provides for the use of the compounds of the invention for the preparation of medicaments for the prevention, amelioration, and/or treatment of ocular diseases associated with oxidative stress.

The invention provides methods for the prevention, amelioration, or treatment of a disease or condition associated with oxidative stress in a subject by administration of a therapeutically effective amount of a compound to the subject to increase the expression or activity of a at least an active fragment of a peroxididase in the subject. The methods include delivery of the compound to an organ, tissue, or cell undergoing oxidative stress. In certain embodiments, the compound is delivered to the eye, for example, to the retina of the eye. Examples of active fragment of the peroxidase include, but are not limited to, the active fragment of a peroxidase selected from the group consisting of glutathione peroxidase (Gpx) 1, Gpx2, Gpx3, Gpx4, Gpx5, Gpx6, Gpx7, Gpx8, and catalase. In certain embodiments, the methods further include administration of a compound to the subject, for example to the eye of the subject, to increase the expression or activity of at least an active fragment of an active oxygen species metabolizing enzyme. Examples of active oxygen species metabolizing enzyme fragment of an active oxygen species metabolizing enzyme include, but are not limited to, superoxide dismutase (SOD) 1, SOD 2, and SOD3.

Methods provided by the invention to increase the expression or activity of the peroxide metabolizing enzyme include delivery of an expression construct to a cell, preferably a retinal cell, for expression of the at least the active fragment of a peroxide metabolizing enzyme operably linked to a promoter sequence. Methods provided by the invention to increase the expression or activity of the active fragment of an active oxygen species metabolizing enzyme include deliver of an expression construct to a cell, preferably a retinal cell, preferably a cell including an expression construct for expression of a peroxidase, for expression of the at least the active fragment of the active oxygen species metabolizing enzyme operably linked to a promoter sequence. The methods provided by the invention include the expression of an active fragment of the peroxidase and an active fragment of the active oxygen species metabolizing enzyme are targeted to a single cellular compartment, such as the cytoplasm, mitochondria, endoplasmic reticulum, or nucleus. In certain embodiments, a first active fragment of the peroxidase is targeted to the cytoplasm of a cell and a first active fragment of the active oxygen species metabolizing enzyme is targeted to a first cellular compartment; and a second active fragment of the peroxidase is targeted to the mitochondria of the cell and the second active fragment of the active oxygen species metabolizing enzyme are targeted to a second cellular compartment. In certain embodiments, the first cellular compartment the mitochondria and the second cellular compartment is the cytoplasm.

The invention provides for expression of various delivery and expression of various proteins in various cellular compartments. For example, the invention provides for expression of the following pairs of proteins in the mitochondria: SOD2 and a mitochondrially targeted catalase, SOD2 and a mitochondrially targeted glutathione peroxidase (any of Gpx1-8), SOD2 and a mitochondrially targeted Gpx4, and SOD2 and a mitochondirally targeted Gpx1; and the following pairs of proteins in the cytosol: SOD1 and catalase, SOD1 and a mitochondirally targeted Gpx; SOD1 and Gpx1; SOD1 and Gpx4. The invention also provides for the expression of any pair of mitochondrially targeted proteins in a cell with any pair of cytoplasmically targeted proteins.

The methods provided by the invention further include the expression of glial cell line-derived neurotrophic factor (GDNF) in a cell, preferably a retinal cell, with one or more of the proteins above. The GDNF can be targeted to the same cellular compartment or a different cellular compartment than the other proteins for expression in the method.

Methods for delivery of the expression constructs of the invention include the use of any viral or non-viral methods known. For example, in the methods of the invention, the expression construct can be provided to the cell in a viral vector selected from the group consisting of an adenoviral (Ad) vector, an adeno-associated viral vector (AAV), a lentiviral vector, and a herpes simplex viral (HSV) vector. Adenoviral associate viral vectors for use in the invention include, but are not limited to, AAV2 viral vectors, hybrid AAV2/4 viral vectors, and hybrid AAV2/5 viral vectors. In certain embodiments, the AAV viral vector is self-complementary. In certain embodiments, the viral vector is replication competent. In certain embodiments, the viral vector is replication incompetent.

The invention provides methods for delivery of the coding sequences for expression of the fragment of one or more active peroxidases and the active fragment of one or more active oxygen species metabolizing enzymes are incorporated into a single expression vector (i.e., polycystronic expression vector). In certain embodiments, methods can include the use of two polycystronic expression vectors each including the coding sequences for two active fragments of enzymes. Such an expression vector can further include an expression construct for GDNF. The invention also provides methods for the delivery of the coding sequences for expression of the active fragment of one or more peroxide metabolizing enzymes and the active fragment of one or more active oxygen species metabolizing enzymes are incorporated into separate expression vectors.

The methods provided by the invention include the use of tissue specific or non-tissue specific (e.g., ubiquitous) promoters. In certain embodiments, expression construct promoter sequence include, but are not limited to, interphotoreceptor retinoid-binding protein (IRBP) promoter, a cytomegalovirus (CMV) promoter, β-globin promoter, cone arrestin promoter, RPE65 promoter, cis-Retinaldehyde-binding protein (CRALBP) promoter is a retinal-pigment-epithelium (RPE)-specific promoter, chicken β-actin (CBA) promoter, and small chicken β-actin (smCBA) promoter.

The methods provided by the invention include methods for directing the proteins expressed by the expression construct to a specific subcellular compartment. The method provides for the preparation and use of active fragments of the peroxide metabolizing enzyme or the active fragments of the active oxygen species metabolizing enzyme, or both being independently operably linked to a signal sequence for targeting to a specific subcellular compartment including, but not limited to, mitochondrial signal sequence, endoplasmic reticulum signal sequence, and nuclear signal sequence. The methods of the invention also provide for the disruption or replacement of signal sequences present in the active fragments of the peroxide metabolizing enzyme or the active fragments of the active oxygen species metabolizing enzyme, or both, to redirect the targeting of the protein in the cell or to prevent the protein from being exported out of the cell.

The methods of the invention provide for ocular administration of the expression constructs of the invention. Preferred methods of delivery include, but are not limited to of subretinal injection and intravitreal injection, for example by using a cannula.

The invention provides methods including further administering one or more antioxidants to the subject. The antioxidant can be delivered locally, i.e., to the eye, or systemically, e.g., either enterally or parenterally, or both.

The methods of the invention may further include identifying a subject prone to or suffering from a disease or condition associated with oxidative stress, particularly oxidative stress in an eye. Methods of the invention may also include monitoring the subject for prevention, amelioration, or treatment of the disease or condition associated with oxidative stress, particularly diseases associated with oxidative stress in the eye. Diseases associated with oxidative stress be prevented, ameliorated, or treated by the methods of the invention include, but are not limited to oxidative stress is involved in many diseases, such as atherosclerosis, Parkinson's disease, heart failure, myocardial infarction, Alzheimer's disease, diabetes, chronic lung disease, diseases associated with mitochondrial dysfunction, and diseases associated with chronic inflammation. Diseases of the eye to be prevented, ameliorated, or treated by the methods of the invention include, but are not limited to retinitis pigmentosa, wet age related macular degeneration, dry age related macular degeneration, diabetic retinopathy, Lebers optic neuropathy, and optic neuritis. The methods of the invention can be used with a subject at essentially any state of disease provided that there are viable retinal cells available to which the expression vectors can be delivered.

Methods for monitoring a subject for prevention, amelioration, or treatment of a disease associated with oxidative stress will depend on the specific disease. Methods for monitoring the subject for prevention, amelioration, or treatment of the disease associated with oxidative stress in the eye include, but are not limited to, monitoring the subject by color vision assessment, ophthalmoscopy after pupil dilation, fluorescein angiography, intraocular pressure assessment, electroretinogram, pupil reflex response assessment, refraction test, retinal photography, visual field test, slit lamp examination, and visual acuity assessment.

The invention further provides compositions for practicing the methods including compounds to increase the expression or activity of a at least an active peroxide metabolizing fragment of a peroxide metabolizing enzyme in an organ, tissue, or cell of a subject, particularly in the eye of the subject. In certain embodiments, the active fragment of the peroxide metabolizing enzyme include, but are not limited to, glutathione peroxidase (Gpx) 1, Gpx2, Gpx3, Gpx4, Gpx5, Gpx6, Gpx7, Gpx8, and catalase.

The invention further comprises compounds to increase the expression or activity of at least an active fragment of an active oxygen species metabolizing enzyme in an organ, tissue, or cell of a subject, particularly in the eye of a subject. In certain embodiments, the active oxygen species metabolizing enzymes include, but are not limited to, superoxide dismutase (SOD) 1, SOD 2, and SOD3. In certain embodiments, a compound to increase the expression or activity of at least an active fragment of an active oxygen species metabolizing enzyme can be combined with a compound to increase the expression or activity of at least an active fragment of a peroxide metabolizing enzyme. In certain embodiments, a compound to increase the expression or activity of at least an active fragment of an active oxygen species metabolizing enzyme can be the same compound as a compound to increase the expression or activity of at least an active fragment of a peroxide metabolizing enzyme

In certain embodiments the compound that increases the expression or activity of the peroxide metabolizing enzyme is an expression construct for expression of the at least the active fragment of a peroxide metabolizing enzyme operably linked to a promoter sequence. In certain embodiments, the agent that increases the expression or activity of the active fragment of an active oxygen species metabolizing enzyme comprises an expression construct for expression of the at least the active fragment of the active oxygen species metabolizing enzyme operably linked to a promoter sequence.

Compositions provided by the invention include expression constructs using of any viral or non-viral methods known. For example, in the methods of the invention, the expression construct can be provided to the cell in a viral vector selected from the group consisting of an adenoviral (Ad) vector, an adeno-associated viral vector (AAV), a lentiviral vector, and a herpes simplex viral (HSV) vector. Adenoviral associate viral vectors for use in the invention include, but are not limited to, AAV2 viral vectors, hybrid AAV2/4 viral vectors, and hybrid AAV2/5 viral vectors. Methods for selection of appropriate vectors depending on the specific cell type(s) that the virus is to be delivered to are well known to those of skill in the art. In certain embodiments, the AAV viral vector is self-complementary. In certain embodiments, the viral vector is replication competent. In certain embodiments, the viral vector is replication incompetent.

The invention provides expression constructs including any known promoter sequence that can promote transcription of a nucleic acid sequence in the specific cell or cell types of choice, for example in an eye cell, preferably a retinal cell. In certain embodiments, promoters for use in the invention include, but are not limited to, interphotoreceptor retinoid-binding protein (IRBP) promoter, a cytomegalovirus (CMV) promoter, a β-globin promoter, cone arrestin promoter, RPE65 promoter, cis-Retinaldehyde-binding protein (CRALBP) promoter is a retinal-pigment-epithelium (RPE)-specific promoter, chicken β-actin (CBA) promoter, and small chicken β-actin (smCBA) promoter.

The compositions of the invention include active fragments of enzymes including signal sequences for directing the proteins expressed by the expression construct to a specific subcellular compartment. The invention provides expression constructs for the expression of active fragments of the peroxide metabolizing enzyme or the active fragments of the active oxygen species metabolizing enzyme, or both being independently operably linked to a signal sequence for targeting to a specific subcellular compartment including, but not limited to, mitochondrial signal sequence, endoplasmic reticulum signal sequence, and nuclear signal sequence. Compositions provided by the invention also include expression construct with an active fragment of an enzyme including a disrupted or replaced of signal sequences present on the active fragments of the peroxide metabolizing enzyme or the active fragments of the active oxygen species metabolizing enzyme, or both, to redirect the targeting of the protein in the cell or to prevent the protein from being exported out of the cell.

The invention provides compositions for delivery of the coding sequences for expression of the fragment of one or more active peroxidases and the active fragment of one or more active oxygen species metabolizing enzymes are incorporated into a single expression vector (i.e., polycystronic expression vector). In certain embodiments, compositions can include the use of two polycystronic expression vectors each including the coding sequences for two active fragments of enzymes. Such an expression vector can further include an expression construct for GDNF. The invention also provides compositions for the delivery of the coding sequences for expression of the active fragment of one or more peroxide metabolizing enzymes and the active fragment of one or more active oxygen species metabolizing enzymes are incorporated into separate expression vectors.

The invention provides for pharmaceutical compositions for intraocular administration including one or more compositions of the invention.

The invention further provides the compositions of the invention including an antioxidant.

The invention provides for the use of any composition of the invention for the preparation of a medicament for the prevention, amelioration, or treatment of a disease or condition associated with oxidative stress, particularly oxidative stress of the eye. Particularly when the disease or condition is associated with oxidative stress of the eye is selected from the group consisting of retinitis pigmentosa, age related macular degeneration, diabetic retinopathy, Lebers optic neuropathy, and optic neuritis.

### BRIEF DESCRIPTION OF THE FIGURES

FIGS. 1A-B. Increased oxidative damage and reduced viability in retinal pigmented epithelial (RPE) cells overexpressing superoxide dimustase 1 (SOD1) or SOD2. Untransfected ARPE 19 cells (control) or those transfected with empty plasmid or plasmid containing an expression construct for glutathione peroxidase 1 (Gpx1), (Gpx4), SOD1, or SOD2 were scraped into lysis buffer 48 hours after transfection. Protein carbonyl content was measured by ELISA and cell viability was measured by MU. There was no significant difference in protein carbonyl content in cells overexpressing Gpx1 or Gpx4 compared to control cells, but it was significantly elevated in cells overexpressing SOD1 or SOD2 (A). Cell viability was not different in cells overexpressing Gpx1 or Gpx4 compared to untransfected cells, but it was significantly reduced in cells overexpressing SOD1 or SOD2 (B). The bars represent the mean (±SEM) calculated from 4 experimental values. *p<0.05; **p<0.01 for difference from untransfected cells by ANOVA with Dunnett's correction for multiple comparisons

FIGS. 2A-B. Glutathione peroxidase 1 (Gpx1) and Gpx4 protect RPE cells from oxidative stress. Twenty-four hours after transfection with an expression construct for glutathione peroxidase 1 (Gpx1), Gpx4, SOD1, or SOD2, RPE cells were treated with 7 mM paraquat, H₂O₂, or hyperoxia for 24 hours. Untranfected RPE cells were treated in the same way to serve as controls. Cell lysates were used to measure protein carbonyl content by ELISA (A) and cell viability by MTT (B). The bars represent the mean (±SEM) calculated from 4 experimental values. Compared to control cells treated with paraquat, cells overexpressing Gpx4 had significantly less protein carbonyl content (A) and greater cell survival (B). Cells overexpressing Gpx1 had greater survival, but no significant difference in carbonyl content. Cells overexpressing SOD1 or SOD2 had significantly more carbonyl content, but no difference in viability. Compared to control cells treated with H₂O₂, cells overexpressing Gpx1 or Gpx4 had significantly less carbonyl content (A) and better viability (B), and cells overexpressing SOD1 or SOD2 had higher carbonyl content and no difference in viability. Compared to control cells treated with hyperoxia, cells Gpx1 or Gpx4 had significantly reduced carbonyl content, but no difference in viability, whereas cells overexpressing SOD1 or SOD2 had increased carbonyl and reduced viability. *p<0.05; **p<0.01 for difference from untransfected control cells by ANOVA with Dunnett's correction for multiple comparisons

FIG. 3. Transgenic mice with doxycycline-inducible expression of glutathione peroxidase 4 (Gpx4). Tetracycline response element (TRE)/Gpx4 mice were generated as described in Methods and crossed with opsin/rtTA transgenic mice to generate Tet/opsin/Gpx4 double transgenic mice. Adult Tet/opsin/Gpx4 mice or littermates lacking one of the transgenes were given drinking water containing (+) or lacking (−) 2 mg/ml doxycycline. After 2 weeks, mice were euthanized and retinal homogenates were assayed for protein concentration; samples containing 50 μg were run in immunoblots for Gpx4. The blots were stripped and reprobed for actin. There was an increase in Gpx4 in the retinas of Tet/opsin/Gpx4 mice treated with doxycycline.

FIGS. 4A-B. Doxycycline-induced expression of Gpx4 in Tet/opsin/Gpx4 double transgenics reduces oxidative damage in the retina. Tet/opsin/Gpx4 double transgenic mice or littermates lacking one of the transgenes (controls) were given drinking water containing or lacking 2 mg/ml of doxycycline for two weeks and then assessed for effects of paraquat (A) or hyperoxia (B) on carbonyl content in the retina. (A) Mice were given an intravitreous injection of 1 μl of PBS containing 0.75 mM paraquat in one eye and 1 μl of PBS in the other eye and after 24 hours the protein carbonyl content in the retina was measured by ELISA. The bars represent the mean (+SEM) calculated from 6 mice in each group. For paraquat-injected eyes, the carbonyl content was significantly less (*p<0.05 by ANOVA with Dunnett's correction) in the retinas of Tet/opsin/Gpx4 mice that received doxycycline compared to retinas of Tet/opsin/Gpx4 mice that did not receive doxycycline or retinas of control mice either treated with doxycycline or not (**p<0.005). Paraquat-injected eyes had greater carbonyl content in the retina than fellow eyes-injected with PBS. (B) Mice were placed in 75% oxygen for weeks and then carbonyl content was measured in the retina. The bars represent the mean (±SEM) calculated from 5 mice in each group. Retinal carbonyl content was significantly less in Tet/opsin/Gpx4 mice treated with doxycycline (*p<0.05) compared to Tet/opsin/Gpx4 mice that did not receive doxycycline or control mice whether or not they received doxycycline (tp<).

FIGS. 5A-E. Induced expression of Gpx4 reduces paraquat-induced thinning of the outer nuclear layer (ONL) of the retina. Tet/opsin/Gpx4 double transgenic mice received drinking water containing or lacking 2 mg/ml of doxycycline and littermate control mice were given normal drinking water. After two weeks, the mice were given an intraocular injection of 1 μl of 0.75 mM paraquat in the left eye and 1 μl of PBS in right eye. After another two weeks of water containing or lacking doxycycline, the mice were euthanized and outer nuclear layer (ONL) thickness was measured as described in Methods. The bars represent the mean (±SEM) calculated from 5 mice in each group. Compared to identical regions of the retina in eyes of control mice injected with PBS (A), those from paraquat-injected eyes of doxycycline-treated Tet/opsin/Gpx4 mice appeared to have a slightly thinner outer nuclear layer and this was confirmed by image analysis (E, *p<0.05, ** by ANOVA with Dunnett's correction for multiple comparisons), but significantly thicker than the ONL from paraquat-injected eyes of Tet/opsin/Gpx4 mice that were not treated with doxycycline (C, **p<0.001) or paraquat-injected eyes of control mice (D, ** p<0.001).

FIGS. 6A-E. Induced expression of Gpx4 reduces hyperoxia-induced thinning of the outer nuclear layer (ONL) of the retina. Tet/opsin/Gpx4 double transgenic mice were placed in 75% O₂ and given drinking water containing or lacking 2 mg/ml of doxycycline. Littermate controls were also placed in 75% oxygen or left in room air. After 2 weeks, the mice were euthanized, 10 μm ocular frozen sections were stained with hematoxylin and eosin, and the ONL thickness was measured as described in Methods. Compared to control mice that remained in room air (A, n=5), the ONL of the same region of the retina from eyes of hyperoxia-treated Tet/opsin/Gpx4 mice (B, n=5) appeared somewhat thinner which was confirmed by image analysis (E, *p<0.05 by ANOVA with Dunnett's correction), but was significantly thicker than the ONL of hyperoxia-exposed Tet/opsin/Gpx4 mice that did not receive doxycycline (C, n=5, **p<0.002) or hyperoxia-treated control mice (D, n=5, **p<0.002).

FIGS. 7A-D. Induced expression of Gpx4 prevents loss of retinal function assessed by electroretinograms (ERGs) after intraocular injection of paraquat. Tet/opsin/Gpx4 double transgenic or littermate control mice were given water containing or lacking 2 mg/ml of doxycycline and after 2 weeks received an intraocular injection of 1 pl of 0.75 mM paraquat in one eye and PBS in the contralateral eye. Scotopic ERGs were performed at 2 and 8 days after injection. At 2 days after injection, all eyes injected with paraquat showed a significant reduction in a-wave (A) and b-wave (C) amplitude compared to eyes injected with PBS. However, at 8 days after injection, paraquat-injected eyes of Tet/opsin/Gpx4 mice that received doxycycline showed a-wave (B) and b-wave (D) amplitudes that were essentially identical to those of PBS-injected eyes, and significantly greater than all other paraquat-injected eyes.

FIGS. 8A-D. Induced expression of Gpx4 prevents hyperoxia-induced loss of retinal function assessed by electroretinograms (ERGs). Tet/opsin/Gpx4 double transgenic or littermate control mice were given water containing or lacking 2 mg/ml of doxycycline and after 2 weeks were placed in 75% oxygen. After another 2 weeks, scotopic ERGs (the points represent the mean±SEM calculated from 6 mice in each group) showed that eyes of Tet/opsin/Gpx4 mice exposed to hyperoxia had significantly greater a-wave (A, C) and b-wave (C, D) amplitudes than Tet/opsin/Gpx4 that did not receive doxycycline or control mice that received water containing or lacking doxycycline.

FIGS. 9A-E. Superoxide dismutase 1 (SOD1) overexpression significantly decreases cone function and cone cell number in rd1^{+/+} mice. Transgenic mice in which the actin promoter drives expression of human SOD1 were crossed with rd1^{+/+} mice and offspring were crossed to obtain rd1^{+/+} mice that carried the Sod1 transgene (Sod1-rd1^{+/+} mice). (A) At postnatal day (P) 25, rd1^{+/+}, and Sod1-rd1^{+/+} mice were euthanized and retinal homogenates were run in western blots using an antibody directed against human SOD1. Immunoblots (Ms) showed strong expression of human SOD1 in Sod1-rd1^{+/+} and no detectable expression in rd1^{+/+} mice. Stripping and reprobing of Ms with an antibody directed against β-actin showed that loading was equivalent. (B) At P25, the mean (±SEM) number of carbonyl adducts determined by enzyme-linked immunosorbent assay of retinal homogenates showed a significant increase in oxidized proteins in Sod1-rd1+/+ mice (n=6) compared to rd1 mice (n=9;*P<5.0×10⁻⁴ by unpaired Student's t-test). (C) At P35, compared to rd1 mice, Sod1-rd1^{+/+} mice appeared to show lower cone density in all four quadrants of the retina by confocal microscopy of peanut agglutinin-stained retinal flat mounts (scale bar=50 μm) and this was confirmed by image analysis (D; *p<2.0×10−4, **p<0.02, ***p<0.002, ****p<0.01 by unpaired Student's t-test). (E) Representative wave forms from photopic electroretinograms (ERGs) done in low background illumination at P25 showed lower b-waves for Sod1-rd1^{+/+} mice than rd1^{+/+} mice and measurements confirmed a significant reduction in mean (±SEM) b-wave amplitude Sod1-rd1^{+/+} mice (*P<0.05 by unpaired Welch's t-test).

FIGS. 10A-C. Rd10^{+/+} mice with inducible increased expression of superoxide dismutase 2 (SOD2) and Catalase in the mitochondria of photoreceptors. (A) Schematic diagram of the TRE/Sod2 and TRE/Catalase transgenes are shown. The tetracycline response element (TRE) was coupled to the full-length cDNA for mouse-Sod2. The ornithine transcarbamylase (OTC) leader sequence, which mediates mitochondrial localization, was ligated to the N terminus cDNA for human Catalase and the peroxisomal localization signal (PLS) was deleted from the C terminus prior to coupling to the TRE. Using these constructs, TRE/Sod2 and TRE/Catalase transgenic mice were generated. (B) Multiple crosses were done to generate TRE/Sod2(+/−)-TRE/Catalase(+/−)-rd10^{+/+} mice and homozygous interphotoreceptor retinol-binding protein promoter/reverse tetracycline transactivator-rd10^{+/+} mice (IRBP/rtTA (+/+)-rd10^{+/+} mice). These two types of mice were crossed to yield four groups of offspring, null-rd10^{+/+}, Sod2-rd10^{+/+}, Catalase-rd10^{+/+}, and Sod2/Catalase-rd10^{+/+} mice for which the genotypes are shown. (C) Nullrd10^{+/+}, Sod2-rd10^{+/+}, Catalase-rd10^{+/+}, and Sod2/Catalase-rd10^{+/+} mice were given normal drinking water or water supplemented with 2 mg/ml of doxycycline between postnatal day (P) 10 and P25. Mice were euthanized and the mitochondrial fractions of retinal homogenates were run in immunoblots using antibodies specific for murine SOD2, human Catalase, and murine cyclooxygenase 4 (COX4), which is known to localize to mitochondria. Background levels of murine SOD2 were seen in retinal mitochondria of all mice, but when treated with doxycycline, only Sod2-rd10^{+/+} and Sod2/Catalase-rd10^{+/+} mice showed a substantial increase in SOD2. Likewise, when treated with doxycycline Catalase-rd10^{+/+} and Sod2/Catalase-rd10^{+/+} showed strong bands for Catalase. Strong bands for COX4 were seen in the retinal mitochondria of all mice.

FIGS. 11A-I. Co-overexpression of superoxide dismutase 2 (SOD2) and Catalase in mitochondria reduce superoxide radicals in the retinas of rd10^{+/+} mice. At postnatal day (P) 35, hydroethidine was injected intraperitoneally into wild-type mice (n=4), null-rd10^{+/+} mice treated with doxycycline between P10 and P35 as described in Materials and Methods (n=4), or Sod2/Catalase-rd10^{+/+} mice treated with doxycycline between P10 and P35 (n=4) and after 18 hours the mice were euthanized and ocular sections were examined by confocal microscopy. Representative sections showed minimal fluorescence in the retinas of wild-type mice (A-C), strong fluorescence primarily in the remaining outer nuclear layer (ONL) and outer plexiform layer of the retinas of null-rd10^{+/+} mice (D-F), and minimal fluorescence in the retinas of Sod2/Catalase-rd10^{+/+} mice (G-I). This demonstrates a marked increase in superoxide radicals in the outer retina of mice after degeneration of rods that is reduced by coexpression of SOD2 and Catalase. Scale bar=20 μm. GCL, ganglion cell layer; INL, inner nuclear layer.

FIG. 12A-B. Increased expression of Catalase and superoxide dismutase 2 (SOD2) significantly reduce carbonyl content in the retinas of postnatal day (P) 50 rd10^{+/+} mice. Starting at P10, the mothers of nullrd10^{+/+}, Sod2-rd10^{+/+}, Catalase-rd10^{+/+}, and Sod2/Catalase-rd10^{+/+} mice and after weaning the mice themselves were treated with doxycycline. Mice were euthanized at P35 or P50 and protein carbonyl content was measured by enzyme-linked immunosorbent assay of retinal homogenates. At P35, the mean (±SEM) carbonyl content per mg retinal protein was significantly greater in Sod2-rd10^{+/+} mice than null-rd10^{+/+}, Catalase-rd10^{+/+}, or Sod2/Catalase-rd10^{+/+} mice (A; *P<0.05; **P<0.01 by Tukey-Kramer test). At P50, the mean (±SEM) carbonyl content per mg retinal protein was significantly less in Sod2/Catalase-rd10+/+ mice compared to null-rd10^{+/+}, Sod2-rd10^{+/+}, or Catalase-rd10^{+/+} mice (B; **P<0.01 by Tukey-Kramer test).

FIGS. 13A-D. Increased expression of superoxide dismutase 2 (SOD2) and Catalase in mitochondria of photoreceptors decreases cone cell death in rd10^{+/+} mice. (A) Fluorescence confocal microscopy of peanut agglutinin (PNA)-stained retinal flat mounts showed little difference in cone cell density in 0.0529 mm² bins 0.5 mm superior to the center of the optic nerve in rd10^{+/+} mice at postnatal day (P) 18 or 35 compared to wild-type mice at P18, but by P50 there was an obvious reduction in cone density in rd10^{+/+} mice. At P18, outer segments were seen in wild-type and rd10^{+/+} mice, but at P35 and P50, rd10^{+/+} mice had flattened inner segments and no outer segments. Scale bar=50 μm. (B) Starting at P10, the mothers of null-rd10^{+/+}, Sod2-rd10^{+/+}, Catalase-rd10^{+/+}, and Sod2/Catalase-rd10^{+/+} mice were treated with doxycycline in their drinking water. After weaning, the mice themselves were treated with doxycycline. At P50, mice were euthanized and fluorescence microscopy of PNA-stained retinal flat mounts in 0.0529 mm² bins 0.5 mm superior, inferior, temporal, and nasal to the center of the optic nerve are shown. Sod2/Catalase-rd10^{+/+} mice appeared to have greater cone density in all four regions of the retina compared to null-rd10^{+/+}, Sod2-rd10^{+/+}, and Catalase-rd10^{+/+} mice. Sod2-rd10^{+/+} mice appeared to have the lowest cone density. Scale bar=50 μm. (C) Quantification of cone density by image analysis in each of the four 0.0529 mm² bins showed that Sod2/Catalase-rd10^{+/+} mice had significantly greater mean (±SEM) cone density than Sod2-rd10^{+/+} mice in the superior, inferior, and nasal quadrants of the retina (*P<0.05, **P<0.01 by Tukey-Kramer test). Sod2/Catalase-rd10^{+/+} mice had significantly greater cone density than null-rd10^{+/+} mice in the inferior and nasal quadrants. Sod2/Catalase-rd10^{+/+} mice had significantly greater cone density than Catalase-rd10 mice in the nasal quadrant. Scale bar=50 μm. (D) Cone density measurements from each of the four quadrants in each mouse were consolidated to provide a single cone density measurement per retina. The mean (±SEM) cone density per retina was significantly greater in P₅₀ Sod2/Catalase-rd10^{+/+} mice compared to null-rd10^{+/+}, Sod2-rd10^{+/+}, or Catalase-rd10^{+/+} mice (**P<0.01 by Tukey-Kramer test).

FIGS. 14A-B. Overexpression of superoxide dismutase 2 (SOD2) and/or Catalase does not prevent rod cell death in rd10^{+/+} mice. Rod cell death leads to progressive thinning of the outer nuclear layer (ONL) in rd10^{+/+} mice. Measurement of ONL thickness of doxycycline-treated mice showed no significant differences by Tukey-Kramer test between null-rd10^{+/+}, Sod2-rd10^{+/+}, Catalase-rd10^{+/+}, and Sod2/Catalase-rd10^{+/+} mice at P25 (A) and P35 (B). The bars show the mean (±SD).

FIGS. 15A-B. Increased expression of superoxide dismutase 2 (SOD2) and Catalase preserves some cone cell function at postnatal day (P) 50 in rd10^{+/+} mice. (A) Scotopic electroretinograms (ERGs) were done at P35 in null-rd10^{+/+}, Sod2-rd10^{+/+}, Catalase-rd10^{+/+}, and Sod2/Catalase-rd10^{+/+} mice treated with doxycycline. The mean (±SEM) b-wave amplitude for four different stimulus intensities is plotted for each of four groups of mice and there were no significant differences. (B) Low background photopic ERGs were done as described in Materials and Methods at P50. Representative waveforms are shown for each of the four groups and illustrate a substantially better waveform in Sod2/Catalase-rd10^{+/+} mice compared to null-rd10^{+/+}, Sod2-rd10^{+/+}, or Catalase-rd10^{+/+} mice. The bars show mean (±SEM) photopic b-wave amplitude, which was significantly higher (**P<0.01 by Tukey-Kramer test) for Sod2/Catalase-rd10^{+/+} mice compared to the other three types of mice.

FIGS. 16A-C. Deficiency of superoxide dismutase 1 (SOD1) increases superoxide radicals in the retinas of rd10^{+/+} mice. (A) Heterozygous Sod1 knockout mice that carried two mutant rd10 alleles (Sod1^{+/-}-rd10^{+/+} mice) were crossed to generate rd10+/+ mice wild type at the Sod1 allele (Sod1^{+/+}-rd10^{+/+} mice), Sod1^{+/-}-rd10^{+/+} mice, and rd10^{+/+} mice deficient in SOD1 (Sod1^{-/-}-rd10^{+/+} mice). (B) Immunoblots of retinal homogenates from postnatal day (P) 25 Sod1^{+/+}-rd10^{+/+} and Sod1^{-/-}-rd10^{+/+} mice showed a strong band for SOD1 in the former and no detectable band for SOD1 in the latter. Stripping and reprobing the blots with an antibody directed against β-actin showed that loading was equivalent. (C) At P25, wild type mice (n=4), Sod1^{+/+}-rd10^{+/+} mice (n=4), and Sod1^{-/-}-rd10^{+/+} mice (n=4) were given two intraperitoneal injections of 20 mg/kg of hydroethidine and after 18 hours they were euthanized and ocular sections were examined by confocal microscopy as described in Methods. There was minimal fluorescence in the retinas of wild type mice (a-c), moderate fluorescence primarily in the remaining outer nuclear layer of the retinas of Sod1^{+/+}-rd10^{+/+} mice (d-f), and strong fluorescence in the retinas of Sod1^{-/-}-rd10^{+/+} mice (g-i). Without injection of hydroethidine, Sod1^{+/+}-rd10^{+/+} mice showed no fluorescence (j-l). Scale bar=50 μm

FIG. 17. Deficiency of superoxide dismutase 1 (SOD1) significantly increases protein carbonyl content in the retinas of postnatal day (P) 40 rd10^{+/+} mice. Sod1^{+/+}-rd10^{+/+} mice and Sod1^{-/-}-rd10^{+/+} mice were euthanized at P40 and protein carbonyl content was measured in retinal homogenates by ELISA. The mean (±SEM) carbonyl content per mg retinal protein was significantly greater in Sod1^{-/-}-rd10^{+/+} mice compared to Sod1^{+/+}-rd10^{+/+} mice (*p<0.05 by unpaired Student's t-test).

FIG. 18. Deficiency of superoxide dismutase 1 (SOD1) accelerates loss of cone cell function in rd10^{+/+} mice. At postnatal day (P) 40, low background photopic ERGs for Sod1^{+/+}-rd10^{+/+} mice and Sod1^{-/-}-rd10^{+/+} mice were done as described in Methods. Representative waveforms are shown for each group and illustrate a substantially better waveform for Sod1^{+/+}-rd10^{+/+} mice compared to Sod1^{-/-}-rd10^{+/+} mice. The bars show mean (±SEM) photopic b-wave amplitude, which was significantly higher for Sod1^{+/+}-rd10^{+/+} mice compared to Sod1^{-/-}-rd10^{+/+} mice (*p<0.005 by unpaired Student's t-test).

FIGS. 19A-B. Generation of rd10^{+/+} mice with increased expression of SOD1 and/or cytoplasmic Gpx4. (A) Transgenic mice carrying a β-actin promoter/human Sod1 transgene or murine cytoplasmic Gpx4 coupled to the tetracycline response element (TRE) were crossed with rd10+/+ mice as described in methods. Multiple crosses were done to generate Sod1(+/−)-TRE/Gpx4(+/−)-rd10^{+/+} mice and homozygous interphotoreceptor retinol binding protein promoter/reverse tetracycline transactivator-rd10^{+/+} mice (IRBP/rtTA(+/+)-rd10^{+/+} mice). These two types of mice were crossed to yield 4 groups of offspring, null-rd10, Sod1-rd10, Gpx4-rd10, and Sod1/Gpx4-rd10 mice for which the genotypes are shown. (B) Null-rd10, Sod1-rd10, Gpx4-rd10, Sod1/Gpx4-rd10 mice were given normal drinking water or water supplemented with 2 mg/ml of doxycycline between postnatal day (P) 10 and P25. Immunoblots (IB) of retinal homogenates showed strong expression of human SOD1 in Sod1-rd10 and Sod1/Gpx4-rd10 mice treated with and without doxycycline. Background levels of murine Gpx4 were seen in all mice, but when treated with doxycycline, only Gpx4-rd10^{+/+} and Sod1/Gpx4-rd10^{+/+} mice showed a substantial increase in Gpx4. Stripping and reprobing of IBs with an antibody directed against β-actin showed that loading was equivalent.

FIG. 20. Co-expression of SOD1 and cytoplasmic Gpx4 in photoreceptors significantly improves cone function at postnatal day (P) 40 in rd10^{+/+} mice. Low background photopic ERGs were done at P40 in doxycycline-treated null-rd10, Sod1-rd10, Gpx4-rd10 and Sod1/Gpx4-rd10 mice and representative waveforms were substantially better in Sod1/Gpx4-rd10 mice compared to null-rd10, Sod1-rd10, or Gpx4-rd10 mice. The bars show mean (±SEM) photopic b-wave amplitude, which was significantly higher for Sod1/Gpx4-rd10 mice compared to the other 3 types of mice, and was significantly lower for Sod1-rd10 mice compared to null-rd10 mice (*p<0.05, **p<0.01 by Tukey-Kramer test).

FIGS. 21A-C. Co-expression of SOD1 and mitochondrial Catalase in photoreceptors does not preserve cone cell function at postnatal day (P) 40 in rd10^{+/+} mice. (A) Transgenic mice carrying β-actin promoter/human Sod1 transgene or human Catalase targeted to mitochondria coupled to the tetracycline response element (TRE) were crossed with rd10^{+/+} mice. Multiple crosses were done to generate Sod1(+/−)-TRE/Catalase(+/−)-rd10^{+/+} mice and homozygous interphotoreceptor retinol binding protein promoter/reverse tetracycline transactivator-rd10^{+/+} mice (IRBP/rtTA(+/+)-rd10^{+/+} mice). These two types of mice were crossed to yield 4 groups of offspring, null-rd10, Sod1-rd10, Catalase-rd10, and Sod1/Catalase-rd10 mice for which the genotypes are shown. (B) Null-rd10, Sod1-rd10, Catalase-rd10, Sod1/Catalase -rd10 mice were given normal drinking water or water supplemented with 2 mg/ml of doxycycline between postnatal day (P) 10 and P25. Immunoblots (IB) of retinal homogenates showed strong expression of human SOD1 in Sod1-rd10 and Sod1/Catalase-rd10 mice treated with and without doxycycline. Catalase-rd10 and Sod1/Catalase-rd10 showed strong bands for Catalase when treated with doxycycline. Stripping and reprobing of IBs with an antibody directed against β-actin showed that loading was equivalent. In IBs of cytosolic and mitochondrial fractions of retinal homogenates, only the cytosolic fraction showed a substantial increase in SOD1 and only mitochondrial fraction showed a substantial increase in Catalase and COX4, which is known to localize to mitochondria. (C) Low background photopic ERGs were done at P40 and representative waveforms were substantially better in null-rd10 mice compared to Sod1-rd10 or Sod1/Catalase-rd10 mice. The mean (±SEM) photopic b-wave amplitude was significantly lower for Sod1-rd10 mice and Sod1/Catalase-rd10 mice compared to null-rd10 mice (*p<0.05, **p<0.01 by Tukey-Kramer test).

### DETAILED DESCRIPTION

### Definitions

“Active fragment” as in “active fragment of an enzyme” is understood as at least that portion of the enzyme that can catalyze the same reaction as the native, full length enzyme (e.g., inactivation of a peroxide, dismutation of superoxide into oxygen and hydrogen peroxide). In an embodiment, the active fragment of the enzyme has at least 50%, 60%, 70%, 80%, 90%, 100%, or more of the activity of the native full length enzyme. Activity can be determined by any of a number of enzyme kinetic parameters known to those of skill in the art, including, but not limited to, rate of product production by the active fragment as compared to the native, full length protein under the same conditions of substrate availability, temperature, etc. Methods to determine active fragments of enzymes is routine and well within the ability of those of skill in the art. Determination of active fragments can be performed initially using sequence alignments and other methods followed by routine enzyme kinetic experiments. Active fragments can include deletions of the amino acid sequence from the N-terminus or the C-terminus, or both. For example, an active fragment can have an N- and/or a C-terminal deletion of about 1, 2, 3, 4, 5, 6, 7, 8, 9, 10, 15, 20, 25, 30, 35, 40, 45, 50, 55, 60, 65, 70, 75, 80, 85, 90, 95, 100, or more amino acids. Active fragments can also include one or more internal deletions of the same exemplary lengths. Active fragments can also include one or more point mutations, particularly conservative point mutations, preferably outside of the catalytic center. At least an active fragment of an enzyme can include the full length, wild-type sequence of the enzyme.

As used herein, “active oxygen species” or “reactive oxygen species” are understood as understood as transfer of one or two electrons produces superoxide, an anion with the form O₂⁻, or peroxide anions, having the formula of O₂²⁻ or compounds containing an O—O single bond, for example hydrogen peroxides and lipid peroxides. Such superoxides and peroxides are highly reactive and can cause damage to cellular components including proteins, nucleic acids, and lipids.

An “agent” is understood herein to include a therapeutically active compound or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein “amelioration” or “treatment” is understood as meaning to lessen or decrease at least one sign, symptom, indication, or effect of a specific disease or condition. For example, amelioration or treatment of retinitis pigmentosa (RP) can be to reduce, delay, or eliminate one or more signs or symptoms of RP including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conjunction with other therapeutic agents and interventions. Amelioration or treatment does not require that the disease or condition be cured.

“Antioxidant” as used herein is understood as a molecule capable of slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an active oxygen species, protein carbonyl content) or a substance produced by a reporter construct (e.g, β-galactosidase or luciferase). Depending on the method used for detection the amount and measurement of the change can vary. Changed as compared to a control reference sample can also include a change in night vision, overall visual acuity, size of visual field, cone density in the retina, thickness of the retina, particularly the outer nuclear layer of the retina, and reduction in a- or b-wave amplitudes on scotopic or ERGs. Determination of statistical significance is within the ability of those skilled in the art.

“Co-administration” as used herein is understood as administration of one or more agents to a subject such that the agents are present and active in the subject at the same time. Co-administration does not require a preparation of an admixture of the agents or simultaneous administration of the agents.

A “conservative amino acid substitution” is one in which the amino acid residue is replaced with an amino acid residue having a similar side chain. For example, families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

“Contacting a cell” is understood herein as providing an agent to a test cell e.g., a cell to be treated in culture or in an animal, such that the agent or isolated cell can interact with the test cell or cell to be treated, potentially be taken up by the test cell or cell to be treated, and have an effect on the test cell or cell to be treated. The agent or isolated cell can be delivered to the cell directly (e.g., by addition of the agent to culture medium or by injection into the cell or tissue of interest), or by delivery to the organism by an enteral or parenteral route of administration for delivery to the cell by circulation, lymphatic, intraocular injection, intravitreal injection, subretinal injection , periocular injection or other means.

As used herein, “detecting”, “detection” and the like are understood that an assay performed for identification of a specific analyte in a sample, a product from a reporter construct or heterologous expression construct (e.g., viral vector) in a sample, or an activity of an agent in a sample. Detection can include the determination of oxidative damage in a cell or tissue, e.g., as determined by protein carbonyl content. Detection can include determination of cell density, particularly specific cell type cell density, cell viability/apoptosis, thickness of the retina, particularly the nuclear layer, photoreceptor function e.g, as determined by electroretinography, etc. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for other signs or symptoms of the disease, disorder, or condition.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the loss of night vision, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

“Expression construct” as used herein is understood as a nucleic acid sequence including a sequence for expression as a polypeptide or nucleic acid (e.g., siRNA, shRNA) operably linked to a promoter and other essential regulatory sequences to allow for the expression of the polypeptide in at least one cell type. In a preferred embodiment, the promoter and other regulatory sequences are selected based on the cell type in which the expression construct is to be used. Selection of promoter and other regulatory sequences for protein expression are well known to those of skill in the art. An expression construction preferably also includes sequences to allow for the replication of the expression construct, e.g., plasmid sequences, virus sequences, etc. For example, expression constructs can be incorporated into replication competent or replication deficient viral vectors including, but not limited to, adenoviral (Ad) vectors, adeno-associated viral (AAV) vectors of all serotypes, self-complementary AAV vectors, and self-complementary AAV vectors with hybrid serotypes, self-complementary AAV vectors with hybrid serotypes and altered amino acid sequences in the capsid that provide enhanced transduction efficiency, lentiviral vectors, or plasmids for bacterial expression.

As used herein, “glial cell line-derived neurotropic factor” or “GDNF” is a protein demonstrated to be effective in reducing oxidative stress in the eye (see, e.g., Dong et al., 2007. J. Neurochem. 103:1041-1052). At least six variants of human GDNF have been identified including GenBank Nos: NM_{—}001145453, NM_{—}145793; NM_{—}005264; NM_{—}199234; NM_{—}199231; and NM_{—}000514 (see also the sequence listing).

As used herein, “heterologous” as in “heterologous protein” is understood as a protein not natively expressed in the cell in which it is expressed, or a protein expressed from a nucleic acid that is not endogenous to the cell. For example, a heterologous protein is a protein expressed from a reporter construct, or a protein present in the cell that is expressed from an expression construct introduced into the cell, e.g. viral vector expression construct.

As used herein, the terms “identity” or “percent identity”, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10 are matched, the two sequences share 90% sequence identity. The identity between two sequences is a direct function of the number of matching or identical positions. Thus, if a portion of the reference sequence is deleted in a particular peptide, that deleted section is not counted for purposes of calculating sequence identity. Identity is often measured using sequence analysis software e.g., BLASTN or BLASTP (available at (www.ncbi.nih.gov/BLAST). The default parameters for comparing two sequences (e.g., “Blast”-ing two sequences against each other), by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1. Additional, computer programs for determining identity are known in the art.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention. For example, a kit can include an expression construct for expression of a peroxidase and/or an active oxygen species metabolizing enzyme in the eye and instructions for use, all in appropriate packaging. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity. Nucleic acid sequences can also be operably linked in tandem in an expression construct such that both polypeptide encoding sequences are transcribed from a single promoter sequence. Alternatively, each nucleic acid sequence encoding a polypeptide can be operably linked to a single promoter sequence.

“Oxidative stress related ocular disorders” as used herein include, but are not limited to, retinitis pigmentosa, macular degeneration including age related macular degeneration (AMD) both wet and dry, diabetic retinopathy, Lebers optic neuropathy, and optic neuritis.

“Peroxidases” or “a peroxide metabolizing enzyme” are a large family of enzymes that typically catalyze a reaction of the form:

ROOR′+electron donor (2e−)+2H+→ROH+R′OH

For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

The glutathione peroxidase family consists of 8 known human isoforms. Glutathione peroxidases use glutathione as an electron donor and are active with both hydrogen peroxide and organic hydroperoxide substrates. Gpx1, Gpx2, Gpx3, and Gpx4 have been shown to be selenium-containing enzymes, whereas Gpx6 is a selenoprotein in humans with cysteine-containing homologues in rodents. Gpx1, NM_{—}000581 and NM_{—}201397; Gpx2, NM_{—}002083; Gpx3, NM_{—}002084; GPx4, NM_{—}001039847.1, NM_{—}001039848.1, NM_{—}002085.3; Gpx5, NM_{—}001509.2, NM_{—}003996.3; Gpx6, NM_{—}182701.1; Gpx7, NM_{—}015696.3; and Gpx8, NM_{—}001008397.2. Each of the GenBank sequence accession numbers and sequences provided therein are incorporated herein by reference in their entirety. Multiple sequence alignments are provided for glutathione peroxidase in Bae et al. 2009, BMC Evolutionary Biology 9:72, incorporated herein by reference, which can be used to identify active fragments of Gpxes and other peroxidases.

Catalase (NM_{—}001752) is also a peroxidase that catalyzes the metabolism of two molecules of hydrogen peroxide to two molecules of water and one molecule of molecular oxygen (O₂). Active fragments of catalase can be determined by sequence alignments and by routine enzymatic testing methods.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop RP. The age of onset of one or more symptoms of the disease can sometimes be determined by the specific mutation. Prevention can include the delay of onset of one or more signs or symptoms of RP and need not be prevention of appearance of at least one sign or symptom of the disease throughout the lifetime of the subject. Prevention can require the administration of more than one dose of an agent or therapeutic.

“Retinitis pigmentosa” or “RP” is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case.

RP is a type of progressive retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

The diagnosis of retinitis pigmentosa relies upon documentation of progressive loss in photoreceptor function by electroretinography (ERG) and visual field testing. The mode of inheritance of RP is determined by family history. At least 35 different genes or loci are known to cause “nonsyndromic RP” (RP that is not the result of another disease or part of a wider syndrome). RP is commonly caused by a mutation in the opsin gene, but can be caused by mutations in a number of other genes expressed systemically or exclusively in the eye.

A “sample” as used herein refers to a biological material that is isolated from its environment (e.g., blood or tissue from an animal, cells, or conditioned media from tissue culture) and is suspected of containing, or known to contain an analyte, such as a virus, an antibody, or a product from a reporter construct. A sample can also be a partially purified fraction of a tissue or bodily fluid. A reference sample can be a “normal” sample, from a donor not having the disease or condition fluid, or from a normal tissue in a subject having the disease or condition (e.g., cells from a subject having a mutation that predisposes the subject to RP vs cells from a subject not having a mutation that predisposes the subject to RP). A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested.

A “signal sequence” or “signal peptide” as used herein is understood as a peptide sequences that direct proteins into appropriate cellular compartments. Signal sequence are present in proteins that are targeted to specific cellular compartments, or can be added onto proteins that are not targeted to the spe Signal sequences may or may not be removed from the peptide after translocation into the appropriate cellular compartment. Examples of signal sequences for translocation into or retention in various compartments include, but are not limited to:

ER import signal: H₃N-MMSFVSLLLVGILFWATEAEQLTKCEVFQ-
ER retention signal: -KDEL-COOH
Mitochondrial import signal: H₃N-MLSLRQSIRFFKPATRTLCSSRYLL-; or

### H₃N-MLFNLRILLNNAAFRNGHNFMVRNFRCGQPLQLGS-; or

H₃N-MVLPR LYTATSRAA-; or H₃N-MV[L,A]L[R]P[R,Q,L]R[K] LYT[R,K,I]A[V]T[I]S[R,G,C]RA[V,G]A[V]- with amino acids listed in [ ] are acceptable substitutions at the amino acid preceded by the [ ].
Nuclear import signal: -PPKKKRKV-
Membrane attachment signal sequence: H₃N-GSSKSKPK-
Other mitochondrial signal sequences are known and discussed, for example, in Giazo and Payne, 2003 (Mol. Ther. 7:720-730, incorporated herein by reference).

“Small molecule” as used herein is understood as a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

A “subject” as used herein refers to living organisms. In certain embodiments, the living organism is an animal. In certain preferred embodiments, the subject is a mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions such as RP and age-related macular degeneration (AMD) is within the ability of those in the art. Subjects suffering from, and suspected of suffering from, a specific disease, condition, or syndrome are not necessarily two distinct groups.

As used herein, “superoxide dismutase” is understood as an enzyme that dismutation of superoxide into oxygen and hydrogen peroxide. Examples include, but are not limited to SOD1, SOD2, and SOD3. SOD1 and SOD3 are two isoforms of Cu—Zn-containing superoxide dismutase enzymes exist in mammals. Cu—Zn-SOD or SOD1, is found in the intracellular space, and extracellular SOD (ECSOD or SOD3) predominantly is found in the extracellular matrix of most tissues. Both enzymes dismutate the superoxide anion into hydrogen peroxide and oxygen with diffusion-limited rate constants (>10⁹ M⁻¹ sec⁻¹), and both are inhibited by cyanide and azide. Human SOD1 is a homodimer with a molecular mass of 32 kDa, and human SOD3 is a tetramer of >135 kDa in vivo. The subunit of each isoform contains one Cu(II) and one Zn(II) atom. The central region of SOD3 (His-96 to Gly-193), which represents an active fragment of SOD3, is homologous to human SOD1 and contains all of the ligands essential for the coordination of the active site Cu(II) and Zn(II) ions. As many diseases have been associated with mutations in SOD genes, SOD proteins have been widely characterized to identify mutations and/or deletions that do or do not disrupt catalytic activity of the proteins. Exemplary SOD sequences are provided in the sequence listing. Further SOD sequences are provided in GenBank including, but not limited to, accession numbers SOD1, NM_{—}000454.4; SOD2, NM_{—}000636.2, NM_{—}001024465.1, NM_{—}001024466.1; and SOD3, NM_{—}003102.2. Each of the GenBank sequence accession numbers and sequences provided therein are incorporated herein by reference in their entirety.

“Therapeutically effective amount,” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the compounds of this invention are defined to include pharmaceutically acceptable derivatives thereof. A “pharmaceutically acceptable derivative” means any pharmaceutically acceptable salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄₊ salts. This invention also envisions the quaternization of any basic nitrogen-containing groups of the compounds disclosed herein. Water or oil-soluble or dispersible products may be obtained by such quaternization.

The compounds of the invention can, for example, be administered by injection, intraocularly, intravitreally, subretinal, intravenously, intraarterially, subdermally, intraperitoneally, intramuscularly, or subcutaneously; or orally, buccally, nasally, transmucosally, directly to a diseases organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose. For ocular administration, especially subretinal administration, the total volume for administration is of substantial concern with the preferred dosage being in the smallest volume possible for dosing. For administration of viral particles, dosages are typically provided by number of virus particles (or viral genomes) and effective dosages would range from about 10³ to 10¹² particles, 10⁵ to 10¹¹ particles, 10⁶ to 10¹⁰ particles, 10⁸ to 10¹¹ particles, or 10⁹ to 10¹⁰ particles. The effective dose can be the number of particles delivered for each expression construct to be delivered when different expression constructs encoding different genes are administered separately. In alternative embodiment, the effective dose can be the total number of particles administered, of one or more types. The methods herein contemplate administration of an effective amount of compound or compound composition to achieve the desired or stated effect.

Frequency of dosing will depend on the agent administered, the progression of the disease or condition in the subject, and other considerations known to those of skill in the art. For example, pharmacokinetic and pharmacodynamic considerations for compositions delivered to the eye, or even compartments within the eye, are different, e.g., clearance in the subretinal space is very low. Therefore, dosing can be as infrequent as once a month, once ever three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly. Dosing may be determined in conjunction with monitoring of one or more signs or symptoms of the disease, e.g., visual acuity, visual field, night visions, etc.

The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition or for prevention of infection, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms (e.g. reduced expression from expression construct).

The term “pharmaceutically acceptable carrier” refers to a carrier that can be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tween® or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, may also be advantageously used to enhance delivery of compounds of the formulae described herein.

The pharmaceutical compositions of this invention may be administered enterally for example by oral administration, parenterally, intraocularly, by inhalation spray, topically, nasally, buccally, or via an implanted reservoir, preferably by oral or vaginal administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases, or buffers to enhance the stability of the formulated compound or its delivery form. The term parenteral as used herein includes intraocular, subcutaneous, intracutaneous, intravenous, intramuscular, intraarticular, intraarterial, intrasynovial, intrasternal, intrathecal, intralesional, and intracranial injection or infusion techniques.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPANs® and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of the invention may be administered topically, e.g., in the form of eyedrops, particularly for administration of antioxidants in conjunction with administration of expression constructs. The pharmaceutical composition will be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Effective dosages of the expression constructs of the invention to be administered may be determined through procedures well known to those in the art which address such parameters as biological half-life, bioavailability, and toxicity.

### Gene Delivery

Compositions and methods for gene delivery to various organs and cell types in the body are known to those of skill in the art. Such compositions and methods are provided, for example in U.S. Pat. Nos. 7,459,153; 7,041,284; 6,849,454; 6,410,011; 6,027,721; and 5,705,151, all of which are incorporated herein by reference. Expression constructs provided in the listed patents and any other known expression constructs for gene delivery can be used in the compositions and methods of the invention.

### Gene Delivery to the Eye

The eye has unique advantages as a target organ for the development of novel therapies and is often regarded as a valuable model system for gene therapy. It is a relatively small target organ with highly compartmentalized anatomy in which it is possible to deliver small volumes of expression vectors for gene delivery, in the context of a viral particle, as nucleic acid alone, or nucleic acid complexed with other agents. It is possible to obtain precise, efficient, and stable transduction of a variety of ocular tissues with attenuated immune responses due to the immune privilege nature of the eye. The risks of systemic side effects for eye procedures are minimal. Further, if only one eye is treated, the untreated eye may serve as a useful control. Gene therapy offers a potentially powerful modality for the management of both rare and common complex acquired disorders (Banibridge, 2008. Gene Therapy 15:633-634, incorporated herein by reference).

Compositions and methods provided herein include the use of gene delivery to the eye for expression of a peroxidase, a superoxide dismutase, or both. In three stage I clinical trials for the treatment of ocular disease, specifically Leber Congenital Amaurosis, an incurable retinal degeneration that causes severe vision loss, gene delivery using an adenoassociated virus administered subretinally has been demonstrated to be safe. Moreover, as a secondary outcome, improvement in visual function was observed in seven of the first nine treated patients. (Bainbridge, 2008. N Engl J. Med. 358:2231-9; Maguire, 2008. N Engl J. Med. 358:2240-8; Miller, 2008. N Engl J. Med. 358:2282-4; Hauswirth, 2008. Hum Gene Ther. September 7; [Epub ahead of print], each incorporated herein by reference) These data demonstrate that gene delivery can be effective for the treatment of an otherwise incurable ocular disease.

The viral vectors used in each of the studies demonstrate that various gene therapy viral vector designs can be useful for gene deliver. Methods of viral vector design and generation are well known to those of skill in the art, and methods of preparation of viral vectors can be performed by any of a number of companies as demonstrated below. Expression constructs provided herein can be inserted into any of the exemplary viral vectors listed below. Alternatively, viral vectors can be generated base on the examples provided below.

For example, in the Bainbridge study, the tgAAG76 vector, a recombinant adeno-associated virus vector of serotype 2 was used for gene delivery. The vector contains the human RPE65 coding sequence driven by a 1400-bp fragment of the human RPE65 promoter and terminated by the bovine growth hormone polyadenylation site, as described elsewhere. The vector was produced by Targeted Genetics Corporation according to Good Manufacturing Practice guidelines with the use of a B50 packaging cell line, an adenovirus-adeno-associated virus hybrid shuttle vector containing the tgAAG76 vector genome, and an adenovirus 5 helper virus. The vector was filled in a buffered saline solution at a titer of 1×10¹¹ vector particles per milliliter and frozen in 1-ml aliquots at −70° C.

Maguire used the recombinant AAV2.hRPE65v2 viral vector which is a replication-deficient AAV vector containing RPE65 cDNA that has been documented to provide long-term, sustained (>7.5 years, with ongoing observation) restoration of visual function in a canine model of LCA2 after a single subretinal injection of AAV2.RPE65. The cis plasmid used to generate AAV2.RPE65 contains the kanamycin-resistance gene, and the transgene expression cassette contains a hybrid chicken β-actin (CBA) promoter comprising the cytomegalovirus immediate early enhancer (0.36 kb), the proximal CBA promoter (0.28 kb), and CBA exon 1 flanked by intron 1 sequences (0.997 kb). To include a Kozak consensus sequence at the translational start site, the sequence surrounding the initiation codon was modified from GCCGCATGT in the original vector to CCACCATGT. The virus was manufactured by The Center for Cellular and Molecular Therapeutics after triple transfection of HEK293 cells and was isolated and purified by microfluidization, filtration, cationexchange chromatography (POROS 50HS; GE Healthcare, Piscataway, N.J.), density gradient ultracentrifugation and diafiltration in PBS. This combination provides optimal purity of the AAV vector product, including efficient removal of empty capsids and residual cesium chloride. A portion of the product was supplemented with PF68 NF Prill Poloxamer 188 (PF68; BASF, Ludwigshafen, Germany) to prevent subsequent losses of vector to product contact surfaces. The purified virus, with or without PF68, was then passed through a 0.22-μm filter using a sterile 60-ml syringe and syringe filter, and stored frozen (−80° C.) in sterile tubes until use. An injection of 1.5×10¹⁰ vector genome of AAV2.hRPE65v2 in a volume of 150 μl of phosphate-buffered saline supplemented with Pluronic F-68 NF Prill Poloxamer 188 was administered into the subretinal space,

The viral vector used by Hauswirth was a recombinant adeno-associated virus serotype 2 (rAAV2) vector, altered to carry the human RPE65 gene (rAAV2-CB^{SB}-hRPE65), that had been previously demonstrated to restore vision in animal models with RPE65 deficiency. The viral vector includes, in order from 5′ to 3′, an inverted terminal repeat sequence (ITR), a CMV immediate early enhancer, a β-actin promoter, β-actin exon 1, β-actin intron 1, β-actin exon 3, wild-type human RPE65 sequence, SV40 poly(A) sequence, and an inverted terminal repeat. The RPE65-LCA viral vector was delivered by subretinal injection (5.96×10¹⁰ vector genomes in 150 μl).

Further AAV vectors are provided in the review by Rolling 2004 (Gene Therapy 11: S26-S32, incorporated herein by reference). Hybrid AAV viral vectors, including AAV 2/4 and AAV2/5 vectors are provided, for example, by U.S. Pat. No. 7,172,893 (incorporated herein by reference). Such hybrid virus particles include a parvovirus capsid and a nucleic acid having at least one adeno-associated virus (AAV) serotype 2 inverted terminal repeat packaged in the parvovirus capsid. However, the serotypes of the AAV capsid and said at least one of the AAV inverted terminal repeat are different. For example, a hybrid AAV2/5 virus in which a recombinant AAV2 genome (with AAV2 ITRs) is packaged within a AAV Type 5 capsid.

Self-complementary AAV (scAAV) vectors have been developed to circumvent rate-limiting second-strand synthesis in single-stranded AAV vector genomes and to facilitate robust transgene expression at a minimal dose (Yokoi, 2007. IOVS. 48:3324-3328, incorporated herein by reference). Self-complementary AAV-vectors were demonstrated to provide almost immediate and robust expression of the reporter gene inserted in the vector. Subretinal injection of 5×10⁸ viral particles (vp) of scAAV.CMV-GFP resulted in green fluorescent protein (GFP) expression in almost all retinal pigment epithelial (RPE) cells within the area of the small detachment caused by the injection by 3 days and strong, diffuse expression by 7 days. Expression was strong in all retinal cell layers by days 14 and 28. In contrast, 3 days after subretinal injection of 5×10⁸ vp of ssAAV.CMV-GFP, GFP expression was detectable in few RPE cells. Moreover, the ssAAV vector required 14 days for the attainment of expression levels comparable to those observed using scAAV at day 3. Expression in photoreceptors was not detectable until day 28 using the ssAAV vector. The use of the scAAV vector in the gene delivery methods of the invention can allow for prompt and robust expression from the expression construct. Moreover, the higher level of expression from the scAAV viral vectors can allow for delivery to of the viral particles intravitreally rather than subretinally.

Various recombinant AAV viral vectors have been designed including one or more mutations in capsid proteins or other viral proteins. It is understood that the use of such modified AAV viral vectors falls within the scope of the instant invention.

Adenoviral vectors have also been demonstrated to be useful for gene delivery. For example, Mori et al (2002. IOVS, 43:1610-1615, incorporated herein by reference) discloses the use of an adenoviral vector that is an E-1 deleted, partially E-3 deleted type 5 Ad in which the transgene (green fluorescent protein) is driven by a CMV promoter. Peak expression levels were demonstrated upon injection of 10⁷ to 10⁸ viral particles, with subretinal injection providing higher levels of expression than intravitreal injection.

Efficient non-viral ocular gene transfer was demonstrated by Farjo et al. (2006, PLoS 1:e38, incorporated herein by reference) who used compacted DNA nanoparticles as a system for non-viral gene transfer to ocular tissues. As a proof of concept, the pZEEGFP5.1 (5,147 bp) expression construct that encodes the enhanced green fluorescent protein (GFP) cDNA transcriptionally-controlled by the CMV immediate-early promoter and enhancer was used. DNA nanoparticles were formulated by mixing plasmid DNA with CK30PEG10K, a 30-mer lysine peptide with an N-terminal cysteine that is conjugated via a maleimide linkage to 10 kDa polyethylene glycol using known methods. Nanoparticles were concentrated up to 4 mg/ml of DNA in saline. The compacted DNA was delivered at a 0.6 μg dose to the vitreal cavity. GFP expression was observed in the lens, retina, and pigment epithelium/choroid/sclera by PCR and microscopy.

Further, a number of patents have been issued for methods of ocular gene transfer including, but not limited to, U.S. Pat. No. 7,144,870 which provides methods of hyaluronic acid mediated adenoviral transduction; U.S. Pat. Nos. 7,122,181 and 6,555,107 which provide lentiviral vectors and their use to mediate ocular gene delivery; U.S. Pat. No. 6,106,826 which provides herpes simplex viral vectors and their use to mediate ocular gene delivery; and U.S. Pat. No. 5,770,580 which provides DNA expression vectors and their use to mediate ocular gene delivery. Each of these patents is incorporated herein by reference.

### Self-Complementary Adenoviral Vectors

Under normal circumstances, AAV packages a single-stranded DNA molecule of up to 4800 nucleotides in length. Following infection of cells by the virus, the intrinsic molecular machinery of the cell is required for conversion of single-stranded DNA into double stranded form. The double-stranded form is then capable of being transcribed, thereby allowing expression of the delivered gene to commence. It has been shown in a number of cell and tissue types that second strand synthesis of DNA by the host cell is the rate-limiting step in expression. By virtue of already being packaged as a double stranded DNA molecule, self-complementary AAV (scAAV) bypasses this step, thereby greatly reducing the time to onset of gene expression.

Self-complementary AAV is generated through the use of vector plasmid with a mutation in one of the terminal resolution sequences of the AAV virus. This mutation leads to the packaging of a self-complementary, double-stranded DNA molecule covalently linked at one end. Vector genomes are required to be approximately half genome size (2.4 KB) in order to package effectively in the normal AAV capsid. Because of this size limitation, large promoters are unsuitable for use with scAAV. Most broad applications to date have used the cytomegalovirus immediate early promoter (CMV) alone for driving transgene expression. However, it has been shown by others that transgene expression with CMV markedly drops off in certain tissue types, such as eye and liver, sometimes as early as two weeks post-injection. A long acting, ubiquitous promoter of small size would be very useful in a scAAV system.

### Nucleic Acid Regulatory Sequences

The invention provides expression constructs that include any regulatory sequences that are functional in the cells in which protein expression is desired, e.g., retinal pigment epithelial (RPE) cells, rod cells, cone cells, etc. For example, cell and tissue specific promoters such as the interphotoreceptor retinoid binding protein (Fei, 1999, J. Biochem. 125:1189-1199, and Liou, 1991, BBRC. 181:159-165, both incorporated herein by reference), cone arrestin promoter (Pickrell, 2004. IOVS. 45:3877-3884, incorporated herein by reference), RPE65 promoter, and cis-Retinaldehyde-binding protein (CRALBP) promoter is a retinal-pigment-epithelium (RPE)-specific promoter (2,265 bp) when administered subretinally in a rAAV vector can be used in the expression constructs of the instant invention. Alternatively, non-tissue specific promoters including viral promoters such as cytomegalovirus (CMV) promoter, and β-actin promoter can be used such as the chicken β-actin (CBA) promoter.

The chimeric CMV-chicken [beta]-actin promoter (CBA) has been utilized extensively as a promoter that supports expression in a wide variety of cells when in rAAV vectors delivered to retina, including in the clinical trials discussed herein. In addition to broad tropism, the present inventors have observed that CBA also has the capacity to promote expression for long periods post infection (Acland, G. M. et al. MoI Then, 2005, 12:1072-1082, incorporated herein by reference). CBA is −1700 base pairs in length, too large in most cases to be used in conjunction with scAAV to deliver cDNAs (over 300 bps pairs in length). CBA is a ubiquitous strong promoter composed of a cytomegalovirus (CMV) immediate-early enhancer (381 bp) and a CBA promoter-exon1-intron1 element (1,352 bp) (Raisler Proc Natl Acad Sci USA. 2002 Jun. 25; 99(13): 8909-8914, incorporated herein by reference). A shortened CBA promoter sequence, the smCBA promoter sequence, has also been described in which the total size of smCBA is 953 bps versus 1714 bps for full length CBA. The smCBA promoter is described in Mah, et al. 2003 (Hum. Gene Ther. 14:143-152, incorporated herein by reference) and Haire, et al. 2006 (IOVS, 2006, 47:3745-3753, incorporated herein by reference).

Other regulatory sequences for inclusion in expression constructs include poly-A signal sequences, for example SV40 polyA signal sequences. The inclusion of a splice site (i.e., exon flanked by two introns) has been demonstrated to be useful to increase gene expression of proteins from expression constructs.

For viral sequences, the use of viral sequences including inverted terminal repeats, for example in AAV viral vectors can be useful. Certain viral genes can also be useful to assist the virus in evading the immune response after administration to the subject.

In certain embodiments of the invention, the viral vectors used are replication deficient, but contain some of the viral coding sequences to allow for replication of the virus in appropriate cell lines. The specific viral genes to be partially or fully deleted from the viral coding sequence is a matter of choice, as is the specific cell line in which the virus is propagated. Such considerations are well known to those of skill in the art.

### Peptide Signal Sequences

In order for proteins, either endogenously or heterologously expressed, to function properly must exist in the appropriate compartment of the cell. As demonstrated herein, the SOD must be co-expressed with a peroxidase in the same cellular compartment, for example either mitochondrial or cytosolic. Similarly, co-expression of a SOD with a peroxidase together in other cellular compartments, e.g., in the endoplasmic reticulum or the nucleus, would also be expected to provide the same benefits as co-expression of the two proteins in any other cellular compartment.

Proteins can be driven into the same compartment of the cell by any of a number of methods. First, proteins that are naturally targeted to the desired cellular compartment(s) can be selected for expression in a cell. Second, one or more proteins can be modified to include a heterologous signal sequence, in place of a native signal sequence or on a protein not having a signal sequence, appropriately attached to the protein, e.g., at the N-terminus of the protein, to direct the desired proteins to be expressed into the same compartment of the cell. Third, one or more proteins can be modified to remove or modify the native signal sequence to retarget the protein to the desired cellular compartment. It is understood that these methods can be used in combination to direct proteins to the appropriate compartment(s) in the cell.

Further, in certain embodiments of the invention the heterologously expressed proteins from the expression constructs can be targeted to various locations within the cell. For example, in an embodiment, the invention includes the delivery of multiple expression constructs to cells for the expression of at least an active fragment of one of each of a cytoplasmic peroxidase, a cytoplasmic superoxide dismutase, a mitochondrial peroxidase, and a mitochondrial superoxide dismutase. In certain embodiments, the expression construct would encode all four enzymes. In other embodiments, two expression constructs including one expressing the cytosolic enzymes and one expressing the mitochondrial enzymes. In yet another embodiment, each enzyme would be present in a separate expression construct. For example, the active fragments of the four enzymes could include the SOD1 and Gpx4 in the cytoplasm and SOD2 and a mitochondrially targeted catalase in the mitochondria. Other combinations are well within the ability of those of skill in the art.

In frame fusion of coding sequences, such as those provided above, to coding sequences for peptides such as active fragments of peroxidases or SODs is well within the ability of those of skill in the art.

### Codon Optimization

Expression construct design and generation can include the use of codon optimization. The degeneracy of the genetic code is well known with more than one nucleotide triplet coding for most of the amino acids, e.g., each leucine, arginine, and serine are encoded by five different codons each. It is possible to design multiple nucleotide sequences that encode a single amino acid sequence. Redesign of a nucleotide sequence without changing the sequence of the polypeptide encoded is well within the ability of those of skill in the art.

### Delivery or Glial Cell Line-Derived Neurotrophic Factor (GDNF)

The present invention also includes delivery of GDNF to the eye in conjunction with either one or more peroxidases, or one or more peroxidases and one or more superoxide dismutases. GDNF was demonstrated by Dong et al. (2007, J. Neurochem. 103:1041-1052) to provide significant preservation of retinal function in response to oxidative damage (e.g., paraquat, FeSO₄, hyperoxia) as compared to knockout mice not expressing GDNF as measured by a number of methods (e.g., electroretinograms, reduced thinning of retinal layers, and fewer apoptotic cells). GDNF can be delivered as a peptide. Alternatively, and preferably, GDNF is delivered by delivery of an expression construct, for example in the context of an expression vector such as a viral vector. The expression vector can be delivered to the eye using methods and doses such as those provided for the delivery of peroxidases and superoxide metabolizing enzymes of the invention.

### Kits

The present invention also encompasses a finished packaged and labeled pharmaceutical product or laboratory reagent. This article of manufacture includes the appropriate instructions for use in an appropriate vessel or container such as a glass vial or other container that is hermetically sealed. A pharmaceutical product may contain, for example, a compound of the invention in a unit dosage form in a first container, and in a second container, sterile water or adjuvant for injection. Alternatively, the unit dosage form may be a solid suitable for parenteral delivery, particularly intraocular delivery.

As with any pharmaceutical product, the packaging material and container are designed to protect the stability of the product during storage and shipment. Further, the products of the invention include instructions for use or other informational material that advise the physician, technician, or patient on how to appropriately prevent or treat the disease or disorder in question. In other words, the article of manufacture includes instructions indicating or suggesting a dosing regimen including, but not limited to, actual doses, monitoring procedures (e.g. visual acuity testing), and other monitoring information.

Specifically, the invention provides an article of manufacture including packaging material, such as a box, bottle, tube, vial, container, sprayer, needle for intraocular administration, envelope and the like; and at least one unit dosage form of a pharmaceutical agent contained within said packaging material, wherein said pharmaceutical agent comprises a compound of the invention, and wherein said packaging material includes instruction means which indicate that said compound can be used to prevent, manage, treat, and/or ameliorate one or more symptoms associated with oxidative stress associated ocular disease by administering specific doses and using specific dosing regimens as described herein.

### Co-Administration of Compounds

The compositions and methods of the invention can be combined with any other composition(s) and method(s) known or not yet known in the art for the prevention, amelioration, or treatment of diseases associated with oxidative stress.

For example, Li et al. (2008, Mol. Ther. 16:1688-1694, incorporated herein by reference) demonstrated that the A small-interfering RNA (siRNA) designed against p22phox efficiently reduced the expression of the protein in the eye when delivered by means of recombinant adeno-associated virus (AAV) vector. Vector treatment inhibited CNV in the mouse when delivered into the subretinal space where RPE cells were transduced, suggesting that NADPH oxidase-mediated ROS production in RPE cells may play an important role in the pathogenesis of neovascular AMD, and that this pathway may represent a new target for therapeutic intervention in AMD, an ocular disease associated with oxidative stress.

Gorbatyuk et al., (2007, Vision Res. 47: 1202-1208, incorporated herein by reference) also used an AAV vector to deliver an siRNA to treat an ocular disease associated with oxidative stress. An AAV-siRNA targeted to mouse rhodopsin delivered into the subretinal space of mice resulted in the reduction of retinal function caused by diminished RHO mRNA and protein content. This level of reduction was suggested to be useful to permit the replacement of endogenous mRNA with siRNA-resistant mRNA encoding wild-type RHO, and if made specific for dominant mutations in rhodopsin could be useful for the treatment of autosomal dominant RP.

Other strategies for uses of siRNA, shRNA, antisense, and other agents for the treatment of diseases related to oxidative stress can be envisioned.

Reactive oxygen species are continuously generated in different cellular compartments and rapidly interact with critical host macromolecules unless they are intercepted. Oral administration of antioxidants is a relatively inefficient way to counter the constant bombardment by ROS. A complementary strategy is to increase expression of components of the endogenous antioxidant defense system. But there are several components of the antioxidant defense system and it is difficult to know which component might be best for a particular application without systematic testing. We have previously demonstrated that superoxide dismutase 1 (SOD1) is an important component of the antioxidant defense system in the retina, because compared to the retinas of wild type mice, those from mice deficient in SOD1 show high basal levels of oxidative damage and more extensive retinal degeneration when challenged by exposure to oxidants (Dong, 2006 J. Cell Physiol. 208:516-526). Transgenic mice with increased expression of SOD1 driven by the β-actin promoter showed partial protection of the retina from severe oxidative stress compared to wild type mice, but also showed increased basal oxidative stress. This study provided proof-of-concept for the overall approach of bolstering the endogenous antioxidant defense system for treatment of oxidative damage-induced retinal degeneration, but left doubt as to whether SOD1 is the best transgene candidate.

The SODs convert superoxide radicals to hydrogen peroxide which is then metabolized by glutathione peroxidases (Gpx) and catalase. In this study, we compared the effects of overexpressing SOD1, SOD2, Gpx1, and Gpx4 in RPE cells exposed to various types of oxidative stress. Cells expressing Gpx4 were particularly well-protected against oxidative stress and therefore the effect of induced expression of Gpx4 in photoreceptors of the retina was also examined.

Retinitis pigmentosa (RP) is a group of diseases in which one of several different mutations results in death of rod photoreceptor cells. The loss of rods results in night blindness, but patients are still able to function well if illumination is adequate. However, once rods die, there is gradual loss of cones accompanied by constriction of visual fields and eventual blindness. If cone death could be prevented in patients with RP, blindness could be averted.

The outer portion of the retina consists solely of photoreceptors, and rods vastly outnumber cones. After rods die, oxygen utilization in the outer retina is reduced, but because choroidal vessels, unlike retinal vessels, are incapable of autoregulation to decrease blood flow when tissue oxygen levels are increased, the oxygen level in the outer retina becomes markedly elevated. (Yu, 2000. IOVS 41: 3999-4006; Yu, 2004. IOVS. 45: 2013-2019.) After rods are eliminated, there is progressive oxidative and nitrosative damage to cones, which are major contributors to their death (Shen, 2005. J Cell Physiol. 203: 457-464; Komeima, 2006. Proc Natl Acad Sci USA 103: 11300-11305). In several models of RP in which rods die from different mutations, exogenous antioxidants slow cone cell death, indicating a potential therapeutic approach in all RP patients despite tremendous heterogeneity in pathogenic mutations (Komeima, 2007. J Cell Physiol 213: 809-815). High levels of antioxidants have also been found useful in retarding the progression of age-related macular degeneration (AMD). However, delivery of antioxidants to the retina is limited by the blood retina barrier. Therefore, high doses of antioxidants are required to provide any result.

When free radicals are generated they interact with the first available acceptor they contact, and for antioxidants to prevent damage to critical molecules, they must be present in sufficiently high concentrations in correct cellular compartments to reduce chance meetings of radicals with those molecules. This is a difficult requirement for exogenous antioxidants that must penetrate into all cellular compartments and maintain high levels at all times. Herein compositions and methods are provided for bolstering the endogenous antioxidant defense system to provide a more efficient approach to be used alone or in a complimentary fashion to systemically or locally administered antioxidants. As demonstrated herein, increasing levels of certain components or combinations of components of the antioxidant defense system in photoreceptors can have positive effects on cone survival in models of RP.

Increased expression of components of the antioxidant defense system is an appealing strategy for treatment of a broad range of retinal degenerations in which oxidative damage plays an important role, e.g. RP, AMD, diabetic retinopathy, Lebers hereditary optic neuropathy, and optic neuritis . . . . By reducing or eliminating the molecules, e.g., superoxides and peroxides, that cause retinal damage rather than addressing the specific mutations that cause the oxidative stress related ocular diseases, diseases of various etilogies can be treated using the compositions and methods provided herein.

There are several components of the antioxidant defense system and the effects of increased expression of various components varies depending upon the cell type and the nature of the oxidative stress. We had previously demonstrated that transgenic mice with increased expression of SOD1 had reduced damage to photoreceptors when challenged with severe oxidative stress, but in unchallenged mice there was higher than normal constitutive oxidative stress resulting in mild reduction in retinal function (Dong, 2006). Herein, we compared the effects of increased expression of SOD1, SOD2, Gpx1, and Gpx4 in cultured RPE cells. Similar to the situation in vivo, increased expression of SOD1 or 2 in RPE cells enhanced oxidative damage in unchallenged cells, however exposure to oxidative stress resulted in greater increases in oxidative damage in cells over-expressing SOD1 or 2 than in control cells, further, overexpression of SOD1 in a RP mouse model rd1^{+/+} resulted in increased retinal damage as compared to untreated animals, demonstrating that the use of SOD1 or SOD2 did not alleviate oxidative stress in the eye. In contrast, RPE cells over-expressing Gpx1 or 4 showed no increase in constitutive oxidative damage and less oxidative damage than control cells when challenged. Further, as demonstrated herein, expression of SOD1 or SOD2 in combination with a peroxidase such as Gpx4 or catalase was found to be useful for the prevention and treatment of RP in mouse models.

Experiments on the effects of over-expressing Gpx4 in photoreceptors in mouse models of oxidative damage-induced retinal degeneration demonstrated an increased expression of Gpx4 in photoreceptors of double transgenic mice and provided strong protection against paraquat- and hyperoxia-induced damage indicated by reduced protein carbonyl content, preservation of retinal function assessed by ERGs, and reduced photoreceptor cell death. These data demonstrate that glutathione peroxidases, particularly Gpx1 and Gpx4 can be used as a therapeutic transgene for treatment of RP and AMD.

It is clear that SOD1 is an important component of the endogenous anti-oxidant defense system in the retina because mice that lack SOD1 are much more susceptible to oxidative stress (Dong, 2006), but that is a different issue than whether its over-expression can provide therapeutic benefits. Without wishing to be bound by mechanism, possible explanation for the paradoxical effects of over-expression of the SODs in RPE cells is that the benefits of reducing superoxide radicals may be negated by increased generation of hydrogen peroxide. There is a hint of this in transgenic mice with increased expression of SOD1, because they have mildly reduced retinal function when not challenged by oxidative stress (Dong, 2006). However, unlike RPE cells in which over-expression of SOD1 or 2 provides increased oxidative stress-induced damage, in the presence of severe oxidative stress retinal function was partially preserved in transgenic mice with increased expression of SOD1 compared to wild type mice. Therefore the effects of over-expressing components of the antioxidant defense system may vary depending upon the cell type and the level of oxidative stress.

Similar benefits were found from over-expressing Gpx1 and Gpx4 in RPE cells, but there are some theoretical advantages that may favor Gpx4. In addition to reducing hydrogen peroxide, alkyl peroxide, and fatty acid peroxide, it also reduces hydroperoxides in lipoproteins, complex lipids and phospholipids (Girotti et al., 1998. J. Lipid Res. 39:1529-1542). Therefore over-expression of Gpx4 can be particularly advantageous in tissues with high content of polyunsaturated fatty acids, such as the photoreceptors. Unlike over-expression of SOD1, which resulted in mild reduction of retinal function, there was no functional deficit in mice over-expressing Gpx4, and marked rescue of retinal function 8 days after intraocular injection of paraquat which is quite remarkable considering the severe insult incurred by intraocular injection of the paraquat (Cingolani, 2006. Free Radic. Biol. Med. 40:660-669). There was some paraquat- and hyperoxia-induced thinning of the ONL in mice over-expressing Gpx4. Therefore, in some subjects, administration of Gpx4, either alone or in combination with SOD1 or SOD2, can act as a therapeutic transgene for retinal degenerations.

The SODs are key defenders against assault from oxidative stress in many tissues, including the retina, where deficiency of SOD1 markedly increases vulnerability to oxidative stress (Dong, 2006). Therefore, we first tested the concept of utilizing the endogenous antioxidant defense system in RP by exploring the effect of increased expression of SOD1 in rd1^{+/+} mice. Rather than protecting cones in rd1^{+/+} mice, overexpression of SOD1 accelerated their loss of function and death. Similar toxic effects were seen when SOD1 or 2 were overexpressed in cultured retinal pigmented epithelial cells (Lu, 2008. epub ahead of print). Without wishing to be bound by mechanism, it appears that excess production of H₂O₂ contributes to the toxic effects of overexpression of the SODs, because coexpression of the cytosolic form of glutathione peroxidase 4 (cGpx4) with SOD1 eliminated its toxicity. Coexpression of cGpx4 with SOD2 did not eliminate SOD2's toxicity, suggesting that it may be necessary to express a peroxide-metabolizing enzyme in the same cellular compartment as an overexpressed SOD to maximize benefit and minimize risk.

Since oxidative stress is particularly severe in mitochondria in hyperoxic tissues and photoreceptors are packed with mitochondria, we decided to target this cellular compartment. In this study, we have demonstrated that increased expression of SOD2 and Catalase in the mitochondria of photoreceptors of rd10^{+/+} mice reduced superoxide radicals and oxidative damage in the retina, provide significant preservation of cone function, and reduced cone cell death. In contrast, overexpression of SOD2 or Catalase alone in the mitochondria of photoreceptors did not significantly reduce oxidative damage or cone cell death.

Various SODs have been overexpressed in other tissues in an attempt to reduce oxidative damage. Overexpression of SOD1 provides protection against oxidative stress in some situations (Przedborski1992. J Neuosci 12:1658-1667; Cadet, 1994. J Neurochem 62:380-383; Schwartz, 1998. Brain Res 789:32-39; Venugopal, 2007. Liver Int 27:1311-1322), but increases the vulnerability of some tissues to other types of oxidative stress. (Elroy-Stein, 1988. Cell 52: 259-267; Rader. 1989. Neurosci LetT. 99: 125-130). Without wishing to be bound by mechanism, tissues with low levels of glutathione peroxidase might be expected to be intolerant to overexpression of SOD1, because an imbalance between SOD1 and glutathione peroxidase can increase levels of H₂O₂ (de Haan, 1996. Hum Mol Genet. 5: 283-292). This may be part of the explanation for the deleterious effects of overexpression SOD1 in models of RP, but it appears that the nature and severity of the oxidative stress is also important, because overexpression of SOD1 reduced oxidative damage from severe oxidative stress (Dong, 2006).

In primary hippocampal neuron cultures, overexpression of SOD1 reduced cyanide toxicity, but increased toxicity from kainic acid or oxygen/glucose deprivation (Zemlyak, 2006. Brain Res 1088: 12-18; Komeima, 2008. Free Radic Biol Med 45: 905-912; Levine, 2002. Free Radic Biol Med 32: 790-796; Buss, 1997. Protein. Free Radical Biol Med 23: 361-366; Lu, 2006. J Cell Physiol 206: 119-125. Dong, 2006; Przedborski, 1992; Cadet, 1994). Interestingly, the combination of increased expression of SOD1 and cyanide induced increased levels of glutathione peroxidase, whereas increased SOD1 and kainic acid did not. Without being bound by mechanism, it appears that the tissue, the type of oxidative stress, and its severity may all influence the impact of overexpression of SOD1.

In mice with experimental allergic encephalomyelitis and optic neuritis and also mice in which the NADH-ubiquinone oxidoreductase complex I of the respiratory chain has been knocked down in retinal ganglion cells, overexpression of SOD2 in ganglion cells reduced ganglion cell death and optic nerve degeneration (Qi, X, 2004. Ann Neurol 56: 182-191; Qi, 2007. IOVS 48: 681-691). This differs from the situation in cones subjected to hyperoxia after death of rods in which we found that overexpression of SOD2 alone increased oxidative damage and failed to improve cone function or survival.

In other studies, mice deficient in SOD3, but not those deficient in SOD1, show increased susceptibility to lung damage from hyperoxia (Yu, 2004. IOVS 45: 2013-2019) and brain damage from ischemia/reperfusion (Sheng, 1999. Neurosci Lett 267: 13-16). Overexpression of SOD3 protected lungs from several types of injury, and it has been postulated that many insults lead to high levels of reactive oxygen species in the interstitial space of lungs, which could best be neutralized by SOD3, which is secreted (Bowler, 2002. Am J Physiol Lung Cell Mol Physiol 282: L719-L726; Rabbani 2005. BMC Cancer 5: 59; Auten, 2006. Am J Physiol Lung Cell Mol. Physiol. 290: L32-L40). Similarly, high levels of reactive oxygen species have been demonstrated in the extracellular space in association with ischemia-reperfusion, and overexpression of SOD3 has provided benefit. However, deficiency of SOD3 does not increase susceptibility of the retina to paraquat or hyperoxia (A. Dong and P.A. Campochiaro, unpublished results), whereas deficiency of SOD1 markedly increases retinal susceptibility to those sources of oxidative stress. However, the ability of any particular SOD or peroxidase isoform to be useful in the methods of the invention may be dependent on the location of the SOD or peroxidase within the cell. Therefore, a retargeted SOD3 may be useful in the compositions and methods of the invention.

However, Sod3 gene transfer may have some potential usefulness for chronic inflammatory conditions affecting the inner retina; while overexpression of SOD3 alone had no significant effect on ganglion cell or axon loss in mice with chronic experimental allergic encephalomyelitis, when combined with overexpression of Catalase, the effects were greater than the effects of overexpression of Catalase alone (Qi, 2007. IOVS 48: 5360-5370). Thus, it appears that the effects of overexpressing SODs can vary considerably depending upon the situation. Our data indicate that overexpression of SOD1 or 2 alone in photoreceptors can exacerbate oxidative damage in cones after rods have degenerated and accelerate retinal degeneration. However, coexpression of SOD2 and Catalase in the mitochondria of photoreceptors strongly promotes cone survival and maintenance of cone function in a model of RP. This suggests that antioxidant gene therapy is a good therapeutic approach for ocular diseases related to oxidative stress including RP, AMD, and diabetic retinopathy, but must be designed and tested carefully before testing in clinical trials

The following examples are provided merely as illustrative of various aspects of the invention and shall not be construed to limit the invention in any way.

### EXAMPLES

### Example 1

### Materials and Methods

### Construction of Expression Plasmids

The pIRES2-EGFP vector (BD Biosciences Clontech, Mountain View, Calif.) was used as the expression vector in RPE cells. The primers for construction were mouse Gpx1: forward: 5′ GCCTCGAGATGTGTGCTGCTCGGCTCTC 3′, reverse: 5′ GCGGATCCTTAGGAGTTGCCAGACTGCT 3′, mouse Gpx4: forward: 5′ GCCTCGAGATGTGTGCATCCCGCGATGA 3′, reverse: 5′ GCGGATCCCTAGAGATAGCACGGCAGGT 3′, mouse Sod1: forward, ATGGCGATGAAAGCGGTGTGC, reverse: 5′ TTACTGCGCAATCCCAATCAC 3′, mouse Sod2, forward: 5′ ATGTTGTGTCGGGCGGCGTGC 3′, reverse; 5′ TCACTTCTTGCAAGCTGTGTA 3′. Fragments of DNA containing full-length murine Gpx1, Gpx4, Sod1 or Sod2 were subcloned into pGEM-T vector (Promega, Madison, Wis.). Each construct was sequenced to confirm the correct sequence and then excised from pGEM-T and ligated into pIRES2-EGFP expression vector. The expression vectors were used in transient transfections in ARPE19 cells (American Type Culture Collection, Manassas, Va.) using Lipofectamin (Invitrogen Corp., Carlsbad, Calif.). Control cells were prepared by transfection with pIRES2-EGFP vector that did not contain an insert.

### Cell Culture

Transfected and control cells were grown in Dulbecco's Modified Eagles's Medium (DMEM) supplemented with 10% fetal bovine serum (FBS), 100 U/ml penicillin and 100 pg/ml streptomycin (all from Invitrogen Corp, Carlsbad, Calif.) at 37° C. and 5% CO₂. Confluent cells were washed and placed in growth medium supplemented with or without 7 mM paraquat (Aldrich, Wilwaukee, Wis.), or 0.5 mM H₂O₂ (Sigma, St. Louis, Mo.) for one day. To expose cells to hyperoxia, cells were grown to confluence in a 25 cm flask, which was filled with 100% oxygen for 1 minute, then the cap was loosened and the flask was returned to the 5% CO₂ incubator. This was repeated twice a day until the cells were scraped into lysis buffer and collected as described previously (Lu, 2006. J Cell Physiol 206: 119-125, incorporated herein by reference).

### Cell Viability

Cells were plated (50,000 cells per well) in 96-well plates and after attachment they were transiently transfected with one of the experimental or control expression vector. The following day the transfected cells were incubated with 7 mM paraquat or 0.5 mM H₂O₂ for 24 hours. The medium was then replaced with normal growth medium. The number of viable cells was determined with the methylthiazoletetrazolium (MTT) cell viability assay kit (American Type Culture Collection, Manassas, Va.), which determines the number of viable cells by bioreduction of MTT into a colored formazan product which is detected by absorbance at 590 nm with a 96 well plate reader.

### ELISA for Protein Carbonyl Content

Cells were scraped into lysis buffer (10 mM Tris-HCl, pH 7.2, 50 mM NaCl, 1 mM EDTA 0.5% Triton X-100). One proteinase inhibitor cocktail tablet (Roche, Indianapolis, Ind.) was added to each 10 ml of lysis buffer. Mouse retina was dissected and placed into lysis buffer. Cells or retinas were vortexed and freeze-thawed three times, centrifuged at 16,000×g for 10 minutes at 4° C., and supernatants were collected and protein concentrations were determined using the BCA protein assay kit (BioRad, Hercules, Calif.). Protein concentrations were adjusted to 4 mg/ml by dilution with TBS and protein carbonyl content was measured by ELISA as previously described (Lu, 2006; Davies, 2001. Free Radic. Biol. Med. 31:181-190, both incorporated herein by reference). Briefly, cell or retinal lysates (15 μl of 4 mg/ml) were incubated with 45 μl of 10 mM 2,4-dinitrophenylhydrazine (DNPH, Sigma, St. Louis, Mo.) in 6 M guanidine-HCl, 0.5 M potassium phosphate, pH 2.5 for 45 minutes at room temperature mixing every 15 minutes. Five μl of each sample was then added to 995 μl of PBS and 200 μl aliquots were added to triplicate wells of a 96-well plate with a MaxiShorp surface (Nalgene Nunc International, Rochester, N.Y.), and incubated overnight at 4° C. Dilutions of oxidized bovine serum albumin (BSA) were also added to triplicate wells to generate a standard curve. Oxidized BSA was prepared and determined as described (Davies, 2001; Levine, 1990. Methods Enzymol. 186:464-478, each incorporated herein by reference). Unbound protein was washed away with PBS (5×300 μl) and nonspecific sites were blocked for 2 hours at 37° C. with 250 μl per well of 0.1% reduced BSA in PBS. After 5 washes with 400 of PBS, the wells were incubated with 200 μl of anti-DNPH mouse monoclonal IgE (1:1000 dilution in PBS with 0.1% reduced BSA and 0.1% TWEEN® 20; Sigma, St. Louis, Mo.) at room temperature for 1 hour with shaking. After 3 washes with PBS, 200 μl of rat anti-mouse monoclonal IgE conjugated to alkaline phosphatase (1:2000 dilution in PBS with 0.1% reduced BSA and 0.1% TWEEN® 20; Southern Biotechnology Associates. Inc, Birmingham, Ala.) was added to each well and incubated at room temperature for 1 hour. After 3 washes with PBS and 3 washes with alkaline phosphatase buffer (100 mM NaCl, 5 mM MgCl₂, 100 mM Tris-HCl, pH 9.5), 200 μl of paranitrophenyl phosphate (pNPP, Sigma, St. Louis, Mo., 2 mg/ml in alkaline phosphatase buffer) was added to each well and incubated at 37° C. for 30 minutes. The absorbance was measured at 405 nm using a 96 well plate reader. The carbonyl content (nmol/mg protein) of cell lysates was calculated using the oxidized BSA standard curve.

Construction of Double Transgenic Mice with Inducible Expression of Gpx4

A 529 by BamHI and Hind III fragment containing full-length murine Gpx4 cDNA was subcloned into pGEM-T vector (Promega, Madison, Wis.) and then excised and ligated into pTRE2 (Clontech, Mountain View, Calif.) containing the tetracycline response element (TRE). After transformation, a clone with correct orientation of the Gpx4 fragment was identified by DNA sequencing. Purified DNA was linearized with Aat II and Spa1 yielding a 2437 by TRE2/Gpx4/13-globin poly A fusion gene. The fusion gene was purified and transgenic mice were generated by Johns Hopkins Transgenic Mouse Core Laboratory. Mice were screened by polymerise chain reaction (PCR) of tail DNA using an upstream primer in the TRE domain (5′ CACGCTGT TTTGACCTCC 3′) and a downstream primer in the Gpx4 domain (5′ GTCTGGCAACTCCTAA 3′). Tail DNA was obtained by digestion of a 1 cm tail segment in 0.4 ml of 50 mM Tris-HCl, pH 7.5. 400 mM NaCl, 20 mM EDTA, and 0.1% sodium dodecyl sulfate with 5 μl of 20 mg/ml proteinase K, at 55° C. Founders of transgenic TRE2/Gpx4 mice were crossed with C57BL/6 mice to obtain independent lines of TRE2/Gpx4 transgenic mice and crossed with homozygous opsin promoter/reverse tetracycline transactivator (opsin/rtTA) transgenic mice that have been previously described (Chang, 2000. IOVS 41:4281-4287; Ohno-Matsui, 2002. Am. J. Pathol. 160:711-719) to yield opsin/rtTA-TRE/Gpx4 (Tet/opsin/Gpx4) double transgenic mice. The expression level of Gpx4 was assessed by Western blots after treatment with 2 mg/ml of doxycycline in drinking water for 2 weeks.

### Western Blots

Retinal lysates containing 50 μg of protein were subjected to SDS-PAGE using 12% polyacrylamide resolving gel (BioRad, Hercules, Calif., USA). After electrophoresis, the slab gel was transferred onto a nitrocellulose membrane (Amersham, Piscataway, N.J., USA). The membrane was incubated with rabbit anti-Gpx4 polyclonal antibody (1:1000, Cayman, Ann Arbor, Mich., USA), followed by incubation with horseradish peroxidase conjugated to goat anti-rabbit IgG (1:2000, Sigma, St. Louis, Mo., USA). Chemiluminescence reaction product was detected using the ECL kit (Amersham, Piscataway, N.J., USA). To assess loading levels of protein, blots were incubated with rabbit anti-actin polyclonal antibody (1:1000, Sigma, St. Louis, Mo., USA), followed by incubation with horseradish peroxidase conjugated to goat anti-rabbit IgG (1: 2000, Sigma, St. Louis, Mo., USA),

### Paraquat Model of Oxidative Damage-Induced Retinal Degeneration

Tet/opsin/Gpx4 mice were tested in the paraquat model of oxidative damage-induced retinal degeneration (Cingolani, 2006) using techniques similar to those previously described (Dong, 2006). Briefly, double hemizygous transgenic mice were given unsupplemented drinking water (controls) or water containing 2 mg/ml of doxycycline and after 2 weeks a 1 μl intraocular injection of 0.75 mM paraquat (Sigma, St Louis, Mo.) was done in the left eye and 1 of PBS was injected in the right eye. Electroretinograms (ERGS) were done 1 and 8 days after injection. After 2 weeks the mice were euthanized and protein carbonyl content was measured in the retinas of some mice while outer nuclear layer thickness was measured in others.

### Hyperoxia-Induced Oxidative Damage

Tet/opsin/Gpx4 mice were tested in a model of hyperoxia-induced retinal degeneration {Yamada, 2001. J. Am. Pathol. 159:1113-1120; Okoye, 2003. J. Neurosci. 23:4164-4172; Dong, 2006). Double hemizygous Tet/opsin/Gpx4 mice from the same litters received unsupplemented water or water containing 2 mg/ml of doxycycline. As an additional control, wild type C57BL/6 mice. All were exposed to 75% oxygen for 2 weeks and then had ERGs and were euthanized for measurement of carbonyl protein content and measurement of outer nuclear layer (ONL) thickness.

### Recording of ERGs

Scotopic ERGs were recorded (Espion ERG; Diagnosys LLL, Littleton, Mass.), as previously described (Okoye, 2003). Briefly, mice were dark adapted overnight and anesthetized with an intraperitoneal injection of ketamine and xylazine. Pupils were dilated with Midrin P consisting of 0.5% tropicamide and 0.5% phenylephrine hydrochloride (Santen Pharmaceutical Co., Osaka, Japan). The mice were placed on a pad heated to 39° C. and platinum loop electrodes were placed on each cornea after application of gonioscopic prism solution (Alcon Laboratories, Fort Worth, Tex.). A reference electrode was placed subcutaneously in the anterior scalp between the eyes, and a ground electrode was inserted into the tail. The head of the mouse was held in a standardized position in a Ganzfeld bowl illuminator that ensured equal illumination of the eyes. Recordings for both eyes were made simultaneously with electrical impedance balanced. The a-wave was measured from the baseline to the negative peak and the b-wave was measured from peak to peak. An average was calculated from 6 measurements at 11 intensity levels of white light ranging from −3.00 to +1.40 log cd-s/m².

### Measurement of Outer Nuclear Layer Thickness

The ONL consists of the cell bodies of photoreceptors and its, thickness provides an assessment of photoreceptor survival. Thickness of the ONL was done as previously described (Okoye, 2003). Briefly, mice were killed and the eyes were removed and embedded in OCT compound. Ten pm frozen sections were cut parallel to 12:00 meridian through the optic nerve and fixed in 4% paraformaldehyde. The sections were stained with hematoxylin and eosin and examined with an Axioskop microscope (Zeiss, Thornwood, N.Y.). Images were digitalized using a three charge coupled device (CCD) color video camera (IK-TU40A, Toshiba, Tokyo, Japan) and a frame grabber. Image-Pro Plus software (Media Cybernetics, Silver Spring, Md.) was used to calculate the area of the ONL. The Images for display were captured with a Nikon microscope equipped with Nikon Digital Still Camera DXM1200.

### Generation of Transgenic Mice.

Mice were treated in accordance with the Association for Research in Vision and Ophthalmology Statement for the Use of Animals in Research and the US National Institutes of Health Guide for the Care and Use of Laboratory Animals. Mice carrying a β-actin promoter/human Sod1 transgene [C57BL/6-TgN(SOD1)₃Cje/J mice, Sod1(+/−) mice] were purchased from Jackson Laboratories (Bar Harbor, Me.) and crossed with rd1+/+ mice in a C57BL/6 background to obtain Sod1(+/−)-rd1+/+ mice. Full-length murine Sod2 cDNA was generated by reverse transcription—PCR of mouse retinal RNA, cloned into Topo TA cloning vector (Invitrogen, Carlsbad, Calif.), and sequenced. The BamHI and HindIII fragment was released from Topo TA vector and ligated into pTRE2 vector (Clontech, Mountain View, Calif.) containing the TRE. After sequencing, a fragment containing TRE, Sod2, and a 1.2 kb β-globin poly A signal was released from pTRE2 to provide the TRE/Sod2 construct that was used to generate transgenic mice in the Johns Hopkins University Transgenic Mouse Core Facility.

The MCAT plasmid, also known as poCAT, which contains human Catalase gene with the ornithine transcarbamylase leader sequence at its 5′ end and without the peroxisomal localization signal at its 3′ end to provide targeting to mitochondria; transgenic mice with ubiquitous expression Catalase in mitochondria have a long lifespan.34 The MCAT construct was ligated into pTRE2. After sequencing, a fragment containing TRE, MCAT, and a 1.2 kb β-globin poly A signal was released from pTRE2 to provide the TRE/Catalase construct that was used to generate transgenic mice in the Johns Hopkins University Transgenic Mouse Core Facility.

Founder mice were mated with C57BL/6 mice to generate founder lines. Mice from each line were crossed with mice from the IRBP/rtTA driver line to generate IRBP/rtTA-TRE/Sod2 and IRBP/rtTA-TRE/Catalase double transgenic mice. Mice from double transgenic lines were given 2 mg/ml in their drinking water and real-time PCR was done to identify IRBP/rtTA-TRE/Sod2 and IRBP/rtTA-TRE/Catalase lines with strong, inducible transgene expression.

### Genotyping of Mice.

Genotyping was done by PCR of tail DNA using the following primers: human Sod1 (forward:5′-CATCAGCCC TAATCCATCTGA-3′, reverse:5′-CGCGACTAACAATCAAAGTGA-3′); TRE/Sod2 (forward:5′-CACGCTGTTTTGACCTCC-3′, reverse:5′-GCTT GATAGCCTCCAGCAAC-3′); TRE/Catalase (forward:5′-TCTGGAGAA GTGCGGAGATT-3′, reverse:5′-AGTCAGGGTGGACCTCAGTG-3′), and IRBP/rtTA (forward:5′-GTTTACCGATGCCCTTGGAATTGACGAGT-3′, reverse:5′-GATGTGGCGAGATGCTCTTGAAGTCTGGTA-3′). To distinguish homozygous rd1, heterozygous rd1, and wild-type mice, the PCR fragment generated with forward, 5′-CATCCCACCT GAGCTCACAGAAAG-3′ and reverse, 5′-GCCTACAACAGAGGAGCTTCTAGC-3′ was digested with DdeI or BsaAI. To distinguish homozygous rd10, heterozygous rd10, and wild-type mice, the PCR fragment generated with forward, 5′-CTTTCTATTCTCTGTCAGCAAAGC-3′ and reverse, 51-CATGAGTAGGGTAAACATGGTCTG-3′ was digested with CfoI.

Mutant rd10 mice with inducible expression of SOD2, Catalase, or both. Rd10^{+/+} mice (Jackson Laboratories, Bar Harbor, Me.) were used in an elaborate mating scheme to generate TRE/Sod2(+/−)-TRE/Catalase(+/−) rd10+/+ mice and IRBP/rtTA(+/−)-rd10^{+/+} mice. These mice were crossed to generate -rd+/+ mice that did not carry either the TRE/Sod2 or TRE/Catalase transgenes, but that which carried only the TRE/Sod2 transgene, or only the TRE/Catalase transgene, or that which carried both the TRE/Sod2 and TRE/Catalase transgenes. Starting at P10, mothers of these mice were given 2 mg/ml of doxycycline in their drinking water. At P21, the mice were separated from their mothers and given drinking water containing 2 mg/ml of doxycycline. Transgene product was measured by immunoblots of retinal homogenates at P25.

### Immunoblots.

For Sod1(+/−)-rd1^{+/+} mice, whole retinas were dissected and placed in 50 μl of lysis buffer (10 mmol/l Tris, pH 7.2, 0.5% Triton X-100, 50 mmol/l NaCl, and 1 mmol/l EDTA) containing a proteinase inhibitor mixture tablet (Roche, Indianapolis, Ind.). After three freeze/thaw cycles and homogenization, samples were microfuged at 14,000 g for 5 minutes at 4° C. and the protein concentration of the supernatant was measured using a Bio-Rad Protein Assay Kit (Bio-Rad, Hercules, Calif.). For all of the other mice, a Mitochondrial Isolation Kit for Tissue (Pierce, Rockford, Ill.) was used according to the manufacturer's instructions to isolate retinal mitochondria. For each sample, 20 μg of protein was resolved by sodium dodecyl sulfate-polyacrylamide gel electrophoresis and transferred to a nitrocellulose membrane (Hybond-ECL; Amersham Biosciences, Piscataway, N.J.). Rabbit polyclonal antihuman SOD1 (1:1,000; Chemicon International, Temecula, Calif.), rabbit polyclonal anti-SOD2 (1:10,000; Abcam, Cambridge, Mass.), or rabbit polyclonal antihuman Catalase (1:2,000; Athens Research Technology, Athens, Ga.) were used as primary antibody. The secondary antibody was a horseradish peroxidase-coupled goat antirabbit IgG (1:1,000; Cell Signaling, Danvers, Mass.). Blots were incubated in SuperSignal Western Pico Lumino/Enhancer solution (Pierce, Rockford, Ill.) and exposed to X-ray film (Eastman-Kodak, Rochester, N.Y.). To assess loading levels of protein, SOD1 blots were stripped and incubated with polyclonal rabbit anti-β-actin antibody (1:5,000; Cell Signaling, Danvers, Mass.) followed by horseradish peroxidase-coupled goat antirabbit IgG and other blots were stripped and incubated with mouse monoclonal anti-COX4 (1:5,000; Abcam, Cambridge, Mass.) followed by horseradish peroxidase-coupled antimouse IgG (1:2,000; Cell Signaling, Danvers, Mass.).

Assessment of Superoxide Radicals with Hydroethidine.

As previously described (Komeima, 2008, Free Radic Biol Med 45: 905-912; and Behrens, 2008. Science 318: 1645-1647, both incorporated herein by reference) in situ production of superoxide radicals was evaluated using hydroethidine, which in the presence of superoxide radicals is converted to ethidium, which binds DNA and emits red fluorescence at ˜600 nm. Briefly, mice were given two 20-mg/kg intraperitoneal injections 30 minutes apart of freshly prepared hydroethidine (Invitrogen, Carlsbad, Calif.) and euthanized 18 hours after injection. Eyes were rapidly removed and 10-μm frozen sections were fixed in 4% paraformaldehyde for 20 minutes at room temperature, rinsed with phosphate-buffered saline (PBS), and counterstained for 5 minutes at room temperature with the nuclear dye Hoechst 33258 (1:10,000; Sigma, St Louis, Mo.). After rinsing in PBS, slides were mounted with Aquamount solution and evaluated for fluorescence (excitation: 543 nm, emission >590 nm) with a LSM 510 META confocal microscope. Images were captured using the same exposure time for each section.

ELISA for protein carbonyl content. Retinas were homogenized in lysis buffer and centrifuged at 16,000 g for 5 minutes at 4° C. and the protein concentration of the supernatant was measured using a Bio-Rad Protein Assay Kit (Bio-Rad). Samples were adjusted to 4 mg/ml by dilution with Trisbuffered saline, and protein carbonyl content was determined by ELISA, as previously described (Komeima, 2006. Proc Natil Acad Sci USA 103: 11300-11305; Lu, 2008 Antioxid Redox Signal, epub ahead of print).

### Measurement of Cone Cell Density.

Cone density was measured as previously described (Komeima, 2006. Proc Natil Acad Sci USA 103:11300-11305, incorporated herein by reference). Briefly, each mouse was euthanized, and eyes were carefully removed and were fixed in 4% paraformaldehyde for 3 hours or over night at 4° C. After washing with PBS, the cornea, iris, and lens were removed. A small triangle cut was made at 12:00 in the retina for future orientation and after four cuts equidistant around the circumference, the entire retina was carefully dissected from the eye cup and any adherent retinal pigmented epithelium was removed. Retinas were placed in 10% normal goat serum in PBS for 30 minutes at room temperature, incubated for 1 hour at room temperature in 1:100 rhodamine-conjugated peanut agglutinin (Vector Laboratories, Burlingame, Calif.) in PBS containing 1% normal goat serum, and flat mounted. The retinas were examined with a Zeiss LSM 510 META confocal microscope (Carl Zeiss, Oberkochen, Germany) with a Zeiss Plan-Apochromat 20×/0.75 NA objective using an excitation wavelength of 543 nm to detect rhodamine fluorescence. Images were acquired in the frame scan mode. The number of cones was determined by image analysis within four 230 mm×230 mm squares located 1 mm (rd1 mice) or 0.5 mm (wild-type and rd10 mice) superior, inferior, temporal, and nasal to the center of the optic nerve. The investigator was masked with respect to experimental group.

### Measurement of ONL Thickness.

ONL thickness was measured, as previously described (Komeima, 2007. J Cell Physiol 213:809-815). Mice were euthanized, a mark was placed at 12:00 at the corneal limbus, and eyes were removed and embedded in optimal cutting temperature compound. Ten-micrometer frozen sections were cut perpendicular to the 12:00 meridian through the optic nerve and fixed in 4% paraformaldehyde. The sections were stained with hematoxylin and eosin, examined with an Axioskop microscope (Zeiss, Thornwood, N.Y.), and images were digitalized using a three-charge-coupled device color video camera (IK-TU40A; Toshiba, Tokyo, Japan) and a frame grabber. Image-Pro Plus software (Media Cybernetics, Silver Spring, Md.) was used to outline the ONL. ONL thickness was measured at six locations, 25% (51), 50% (S2), and 75% (S3) of the distance between the superior pole and the optic nerve and 25% (I1), 50% (I2), and 75% (I3) of the distance between the inferior pole and the optic nerve.

### Recording of ERGs.

An Espion ERG Diagnosys machine (DiagnoSYS LLL, Littleton, Mass.) was used to record ERGs as previously described (Komeima, 2006. Proc Natil Acad Sci USA 103: 11300-11305; Komeima, 2007. J Cell Physiol 213: 809-815; Okoye, 2003. Neurosci 23: 4164-4172; Ueno, 2008. J Cell Physiol 217: 13-22). For scotopic recordings, mice were adapted to dark overnight, and for photopic recordings, mice were adapted to background white light at an intensity of 30 cd/m² for 10 minutes. The mice were anesthetized with an intraperitoneal injection of ketamine hydrochloride (100 mg/kg body weight) and xylazine (5 mg/kg body weight). Pupils were dilated with Midrin P containing of 0.5% tropicamide and 0.5% phenylephrine, hydrochloride (Santen Pharmaceutical, Osaka, Japan). The mice were placed on a pad heated to 39° C. and platinum loop electrodes were placed on each cornea after application of Gonioscopic prism solution (Alcon Labs, Fort Worth, Tex.). A reference electrode was placed subcutaneously in the anterior scalp between the eyes and a ground electrode was inserted into the tail. The head of the mouse was held in a standardized position in a ganzfeld bowl illuminator that ensured equal illumination of the eyes. Recordings for both eyes were made simultaneously with electrical impedance balanced. Scotopic ERGs were recorded at six intensity levels of white light ranging from −3.00 to 1.40 log cd-s/m². Six measurements were averaged at each flash intensity. Low background photopic ERGs were recorded at 1.48 log cd-s/m² under a background of 10 cd/m². Sixty photopic measurements were taken and the average value was recorded.

### Statistical Analysis

Statistical comparisons were done using ANOVA with Dunnett's test for multiple comparisons, or by using Tukey-Kramer's test for multiple comparisons and unpaired Student's t-test or Welch's t-test for two comparisons, as noted. Differences were judged statistically significant at P<0.05 or P<0.01, as noted.

### Example 2

### Increased Expression of Gpx1 or Gpx4 in RPE Cells Provides Superior Protection Against Oxidative Stress Compared to Increased Expression of SOD1 or SOD2

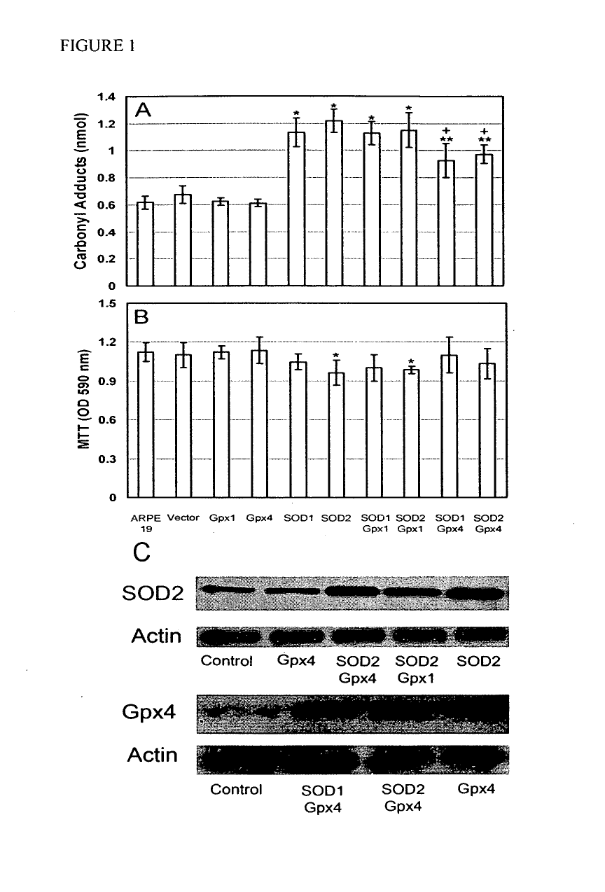

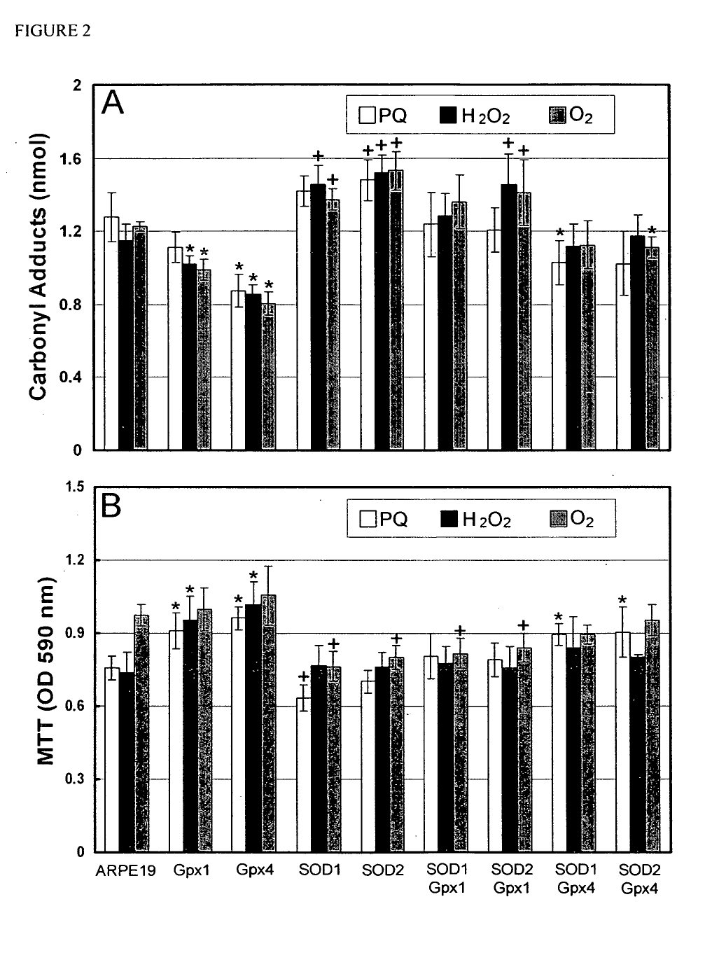

Measurement of the carbonyl content of proteins by ELISA provides a good quantitative assessment of oxidative damage. Compared to control RPE cells, those over-expressing Gpx1 or Gpx4 showed similar protein carbonyl content, but those over-expressing SOD1 or SOD2 showed a significant increase in carbonyl content and reduced viability (FIG. 1). This suggests that increased levels of SOD1 or SOD2 enhance constitutive oxidative damage and reduce cell survival in RPE cells. Control RPE cells that were challenged with paraquat, hydrogen peroxide, or hyperoxia had carbonyl levels in the range of 1.2 nM, compared to 0.6 nM in unchallenged cells. In the presence of all 3 types of oxidative stress, RPE cells over-expressing Gpx4 had significantly less carbonyl content than control RPE cells (FIG. 2). Cells over-expressing Gpx1 had significantly less carbonyl content than control cells in the presence of hydrogen peroxide or hyperoxia, but not paraquat. In contrast, cells over-expressing SOD1 or SOD2 showed increased carbonyl levels compared to control RPE when challenged with each of the 3 types of oxidative stress.

There was a rough, but not exact, correlation between level of oxidative damage assessed by carbonyl content and cell viability. Cells over-expressing Gpx1 or Gpx4 had increased viability compared to control RPE cells when exposed to paraquat or hydrogen peroxide, but not hyperoxia, while cells over-expressing SOD1 or 2 had a significant reduction in viability only in the presence of hyperoxia. These data demonstrate that increased levels of Gpx4 and Gpx1 in RPE cells bolster the antioxidant defense system, while increased levels of SOD1 and SOD2 do not.

### Example 3

### Increased Expression of Gpx4 in Photoreceptors Reduces Paraquat- and Hyperoxia-Induced Oxidative Damage

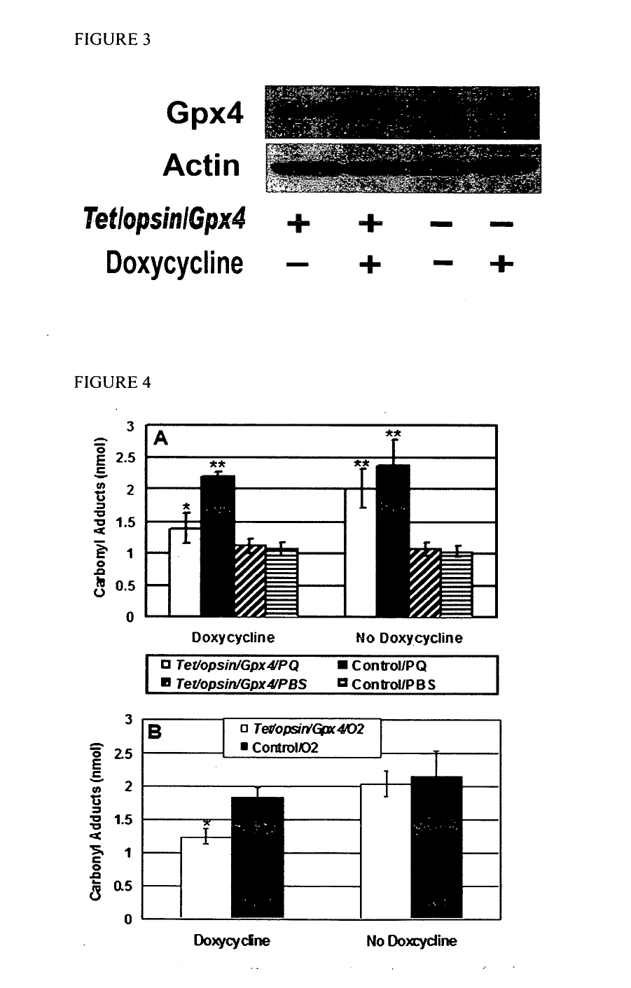

The protective effects of Gpx1 and Gpx4 were quite similar in RPE cells; therefore, it was decided to only investigate the effects of Gpx4 in vivo in photoreceptors. TRE/murine Gpx4 transgenic mice were generated and crossed with opsin/rtTA mice to generate opsin/rtTA-TRE/Gpx4 (Tet/opsin/Gpx4) double transgenic mice. When these mice were given drinking water containing 2 mg/ml of doxycycline for two weeks, immunoblots showed increased levels of Gpx4 in the retina (FIG. 3). When 1 μl of 0.75 mM paraquat was injected into the vitreous cavity of littermate control mice or doxycycline-treated Tet/opsin/Gpx4 mice the protein carbonyl content in the retina was increased compared to mice injected with PBS, but the latter had significantly lower levels than the former (FIG. 4A). In contrast, Tet/opsin/Gpx4 mice that were not treated with doxycycline had similar paraquat-induced elevation of protein carbonyl levels in the retina compared to littermate control mice. When placed in 75% hyperoxia for 2 weeks, Tet/opsin/Gpx4 mice that were treated with doxycycline had significantly lower protein carbonyl content in the retina than doxycycline-treated littermate control mice; however, Tet/opsin/Gpx4 mice that were not treated with doxycycline had similar hyperoxia-induced elevation of protein carbonyl levels in the retina compared to littermate control mice (FIG. 4B).

### Example 4

### Increased Expression of Gpx4 in Photoreceptors Reduces Paraquat- and Hyperoxia-Induced Thinning of the Outer Nuclear Layer (ONL)

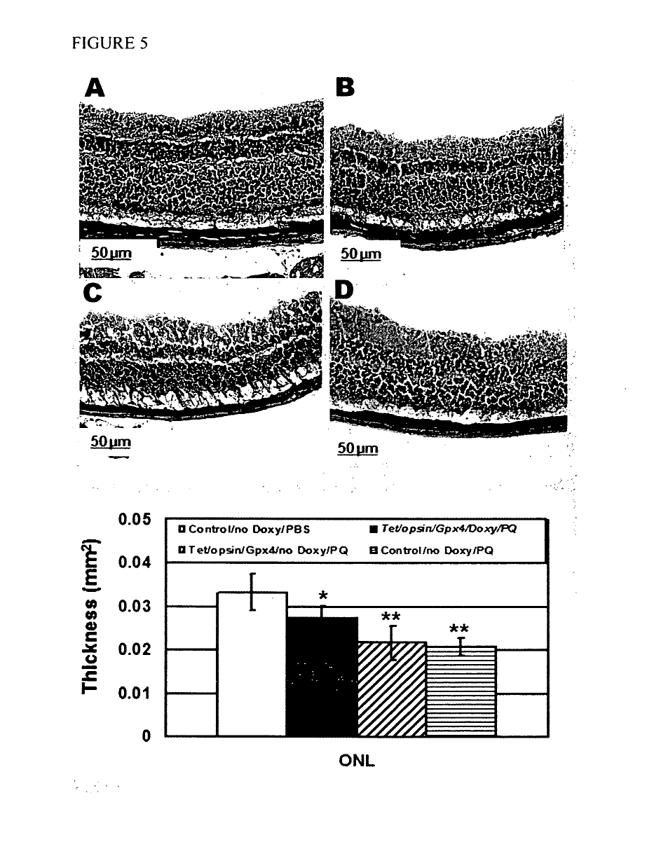

The ONL of the retina contains the cell bodies of the photoreceptors and death of photoreceptors results in thinning of the ONL. Two weeks after intraocular injection of 1 μl of 0.75 mM paraquat, Tet/opsin/Gpx4 mice that were treated with doxycycline had significantly thicker ONLs than Tet/opsin/Gpx4 mice that were not treated with doxycycline or doxycycline-treated littermate control mice (FIG. 5). The protection of photoreceptors by induced expression of Gpx4 was partial, because ONL thickness was significantly less in paraquat-injected Tet/opsin/Gpx4 mice that were treated with doxycycline than in PBS-injected littermate control mice.

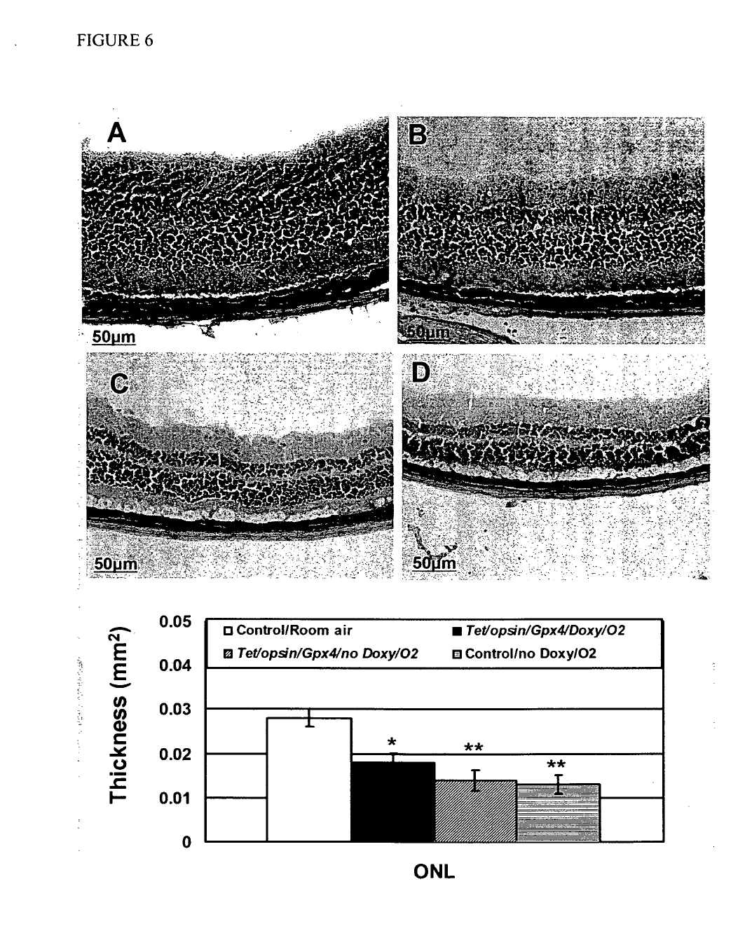

After 2 weeks in 75% oxygen, Tet/opsin/Gpx4 mice that were treated with doxycycline had significantly thicker ONLs than Tet/opsin/Gpx4 mice that were not treated with doxycycline or doxycycline-treated littermate control mice (FIG. 6). The protection of photoreceptors by induced expression of Gpx4 was partial, because ONL thickness was significantly less in hyperoxia-exposed Tet/opsin/Gpx4 mice that were treated with doxycycline than in littermate controls that were not exposed to hyperoxia.

### Example 5

### Increased Expression of Gpx4 in Photoreceptors Reduces Loss of Retinal Function after Injection of Paraquat or Exposure to Hyperoxia

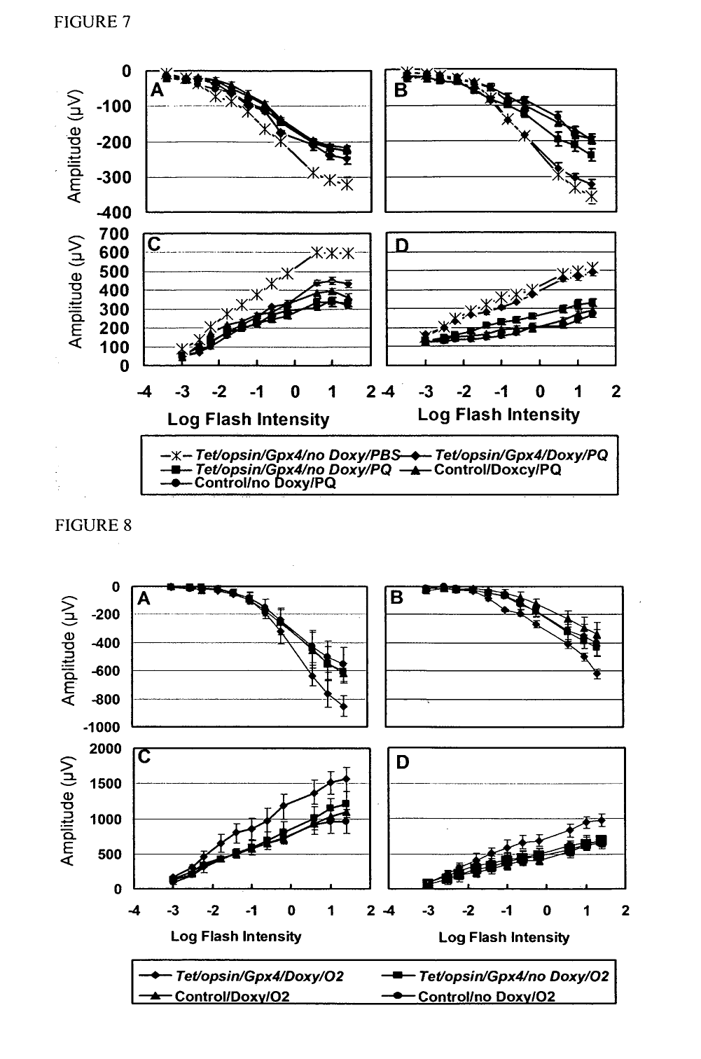

ERGs provide a global assessment of retinal functioning. One day after injection of 1 μl of 0.75 mM paraquat, all mice injected with paraquat showed significantly reduce ERG a- and b-wave amplitudes compared to mice injected with PBS (FIGS. 7A and C). However, 8 days after paraquat injection Tet/opsin/Gpx4 mice that were treated with doxycycline had a- and b-wave amplitudes that were significantly greater than those seen in littermate controls or Tet/opsin/Gpx4 mice that were not treated with doxycycline, and were no different from those seen in mice that had been injected with PBS (FIGS. 7B and D). After 2 weeks in 75% oxygen, Tet/opsin/Gpx4 mice that were treated with doxycycline had a- and b-wave amplitudes that were significantly greater than those seen in littermate controls or Tet/opsin/Gpx4 mice that were not treated with doxycycline (FIG. 8).

### Example 6

### Paradoxical Effect of Overexpression of SOD1 in rd1^{+/+} Mice

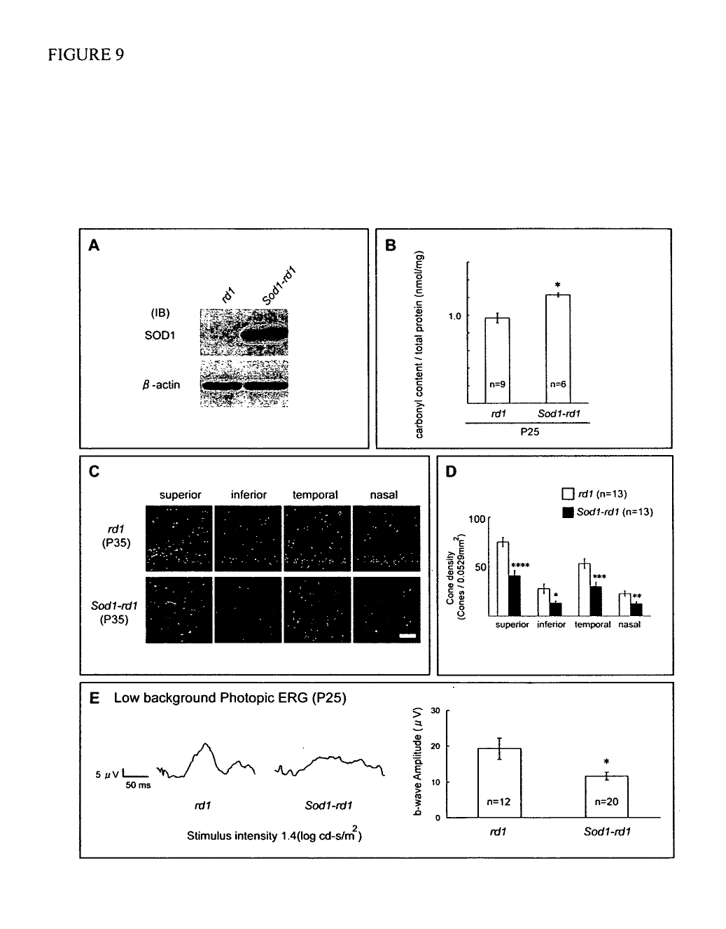

In order to determine if increased levels of superoxide dismutase 1 (SOD1) could slow or prevent cone cell death in a primary rod cell degeneration, transgenic mice in which the actin promoter drives expression of human SOD1 were crossed with rd1^{+/+} mice and offspring were crossed to obtain rd1^{+/+} mice that carry the Sod1 transgene (Sod1-rd1^{+/+} mice). At postnatal day (P) 25, there was strong expression of human SOD1 in Sod1-rd1^{+/+} mice and no detectable expression in rd1^{+/+} mice (FIG. 9a), but surprisingly Sod1-rd1^{+/+} mice showed significantly greater carbonyl adducts on proteins in the retina than did rd1^{+/+} mice, indicating increased rather than decreased oxidative damage (FIG. 9b). At P35, compared to rd1^{+/+} mice, Sod1-rd1^{+/+} mice showed reduced cone density in all four quadrants of the retina (FIG. 9c,d). There was also a reduction in mean photopic b-wave amplitude in P35 Sod1-rd1^{+/+} mice compared to rd1^{+/+} mice, indicating that loss of cone cell function was accelerated by overexpression of SOD1 in rd1^{+/+} mice.

### Example 7

### Generation of Transgenic Mice with Inducible Expression of SOD2, Catalase, or Both

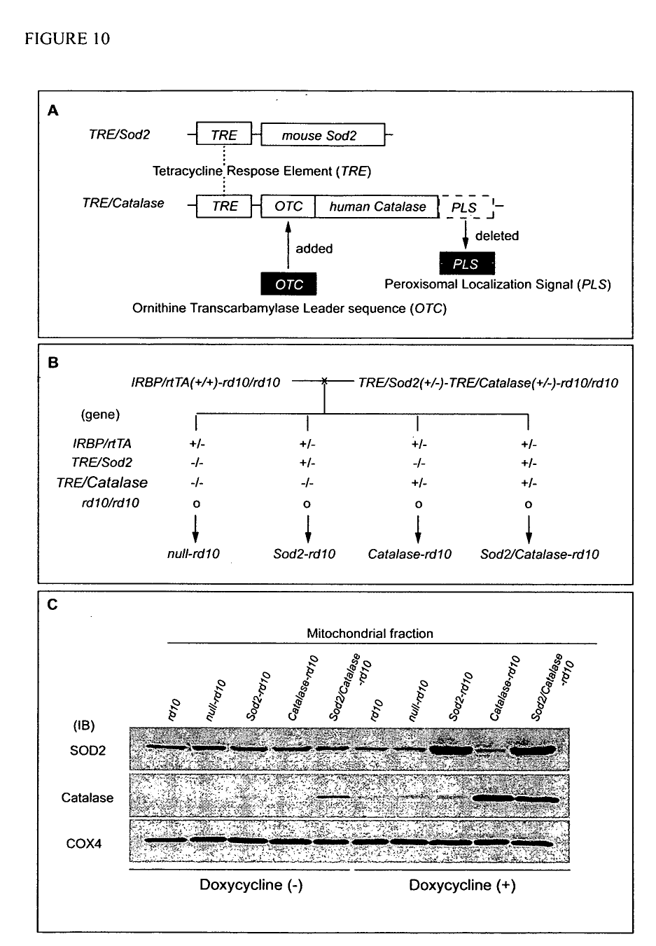

We have previously used the tet/on inducible system to test the effects of overexpressing many different proteins in photoreceptors (Ohno-Matsui, 2002. Am J Pathol 160: 711-719; Okoye, 2003. J Neurosci 23: 4164-4172; Oshima, 2005. FASEB J 19: 963-965; Dong, 2007. J Neurochem 103:1041-1052; Lu, 2008. Antioxid Redox Signal. epub ahead of print; each incorporated herein by reference). To explore the effects of overexpressing components of the antioxidant defense system, we generated tetracycline response element (TRE)/Sod2 mice and TRE/Catalase mice. The peroxisomal targeting signal was deleted from the Catalase transgene and an ornithine transcarbamylase signal sequence was added to direct the Catalase to mitochondria (FIG. 10a). The reverse tetracycline transactivator/interphotoreceptor retinol-binding protein promoter (rtTA/IRBP) was used as the driver line, because it directs expression in both rods and cones. Rd10^{+/+} mice were used for these experiments, because retinal degeneration occurs more slowly in rd10^{+/+} mice than rd1^{+/+} mice. Mice homozygous at both the rtTA/IRBP and rd10 alleles were generated and crossed with mice homozygous at the rd10 allele, but heterozygous at the TRE/Sod2 and TRE/Catalase alleles and the possible offspring are shown in FIG. 10b.

The offsprings were genotyped and after weaning they were given normal drinking water or drinking water containing 2 mg/ml of doxycycline, and then mitochondrial fractions of retinal homogenates were run in immunoblots. A fairly consistent baseline level of murine SOD2 was seen in all samples except those from doxycycline-treated mice that carried the TRE/Sod2 transgene (FIG. 10c). Likewise, strong bands for human Catalase were seen only in samples from doxycycline-treated mice that carried the TRE/Catalase transgene. All samples showed similar bands for COX4, which is expressed in mitochondria, indicating that roughly equivalent amounts of mitochondrial fractions had been loaded. These data demonstrate that mice with either inducible expression of SOD2, Catalase, or both in the mitochondria of photoreceptors had been generated.

### Example 8

### Rd10^{+/+} Mice with Induced Expression of Sod2 and Catalase in Photoreceptors Show Reduced Superoxide Radicals in the Retina

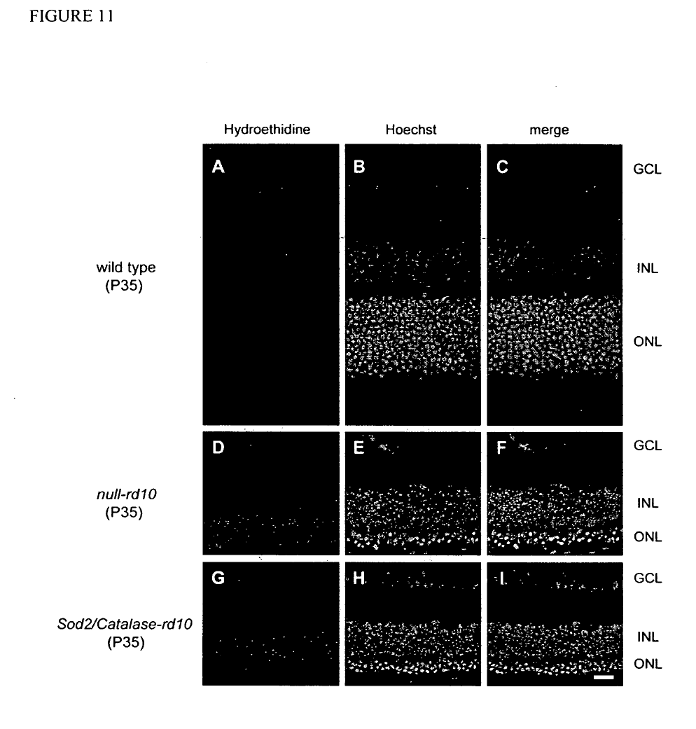

Hydroethidine is taken up into cells and in the presence of superoxide radicals is converted to ethidium, which binds DNA and emits red fluorescence providing a means to visualize production of superoxide radicals in situ (Pietch, 2003. Cardiovasc Res 57: 456-467). We previously utilized this technique to show that there is a striking increase in superoxide radicals in the outer retinas of P30 rd1+/+ mice in which rods have degenerated (Komeima, 2008. Free Radic Biol Med 45: 905-912). At P35, wild-type mice showed minimal fluorescence in the retina when hydroethidine had been injected prior to death (FIG. 11a) indicating low levels of superoxide radicals, but P35 rd10^{+/+} mice showed strong fluorescence in the outer retina indicating high levels of superoxide radicals (FIG. 11b). In contrast, P35 rd10^{+/+} mice with coexpression of SOD2 and Catalase in the mitochondria of photoreceptors showed little fluorescence in the retina when hydroethidine had been injected prior to death (FIG. 11c), indicating a large increase in the capacity to scavenge superoxide radicals.

### Example 9

### Increased Expression of Catalase and SOD2 Significantly Reduce Carbonyl Content in the Retinas of rd10^{+/+} Mice

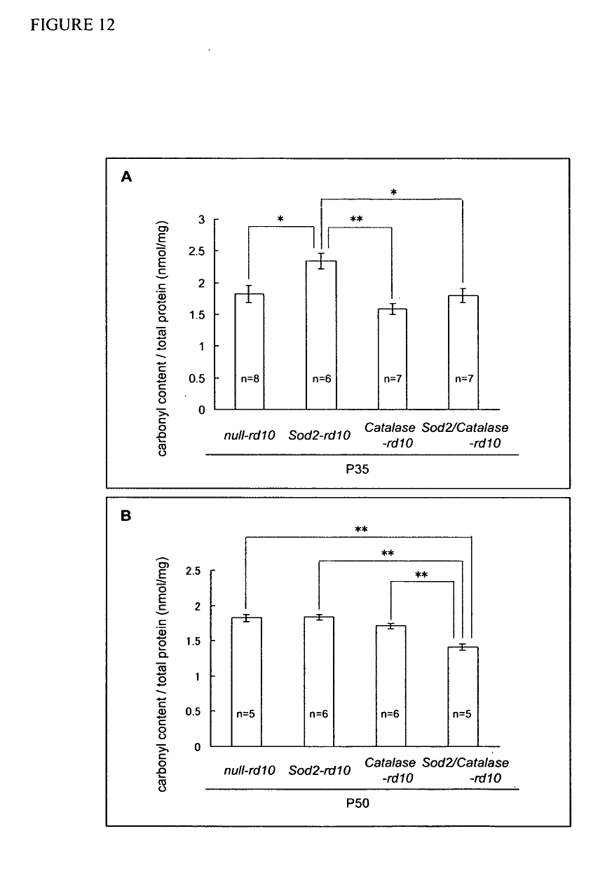

When proteins undergo oxidative damage, the most common modification is introduction of carbonyl groups into side chains (Levine, 2002. Free Radic Biol Med 32: 790-796, incorporated herein by reference), and enzyme-linked immunosorbent assay (ELISA) for carbonyl adducts provides a quantitative measure of oxidative damage (Buss, 1997. Free Radical Biol Med 23: 361-366; Lu, 2006. J Cell Physiol 206: 119-125, both incorporated herein by reference). To determine if the increased capacity to neutralize superoxide radicals translated into protection from oxidative damage we measured carbonyl levels in the retina by ELISA. At P35, a time point when rod degeneration is just being completed in rd10^{+/+} mice, there was no difference in carbonyl levels in the retinas of mice with increased expression of Catalase or both Catalase and SOD2 that did not have increased expression of an antioxidant enzyme (FIG. 12a). However, Sod2-rd10^{+/+} mice had significantly greater carbonyl content per mg retinal protein than null-rd10^{+/+}, Catalase-rd10^{+/+}, or Sod2/Catalase-rd10^{+/+} mice, indicating that increased production of SOD2 in photoreceptors increased oxidative damage in rd10^{+/+} mice. At P50, when cones have been present with no surrounding rods for ˜2 weeks, carbonyl content per mg retinal protein was significantly less in Sod2/Catalase-rd10+/+ mice compared to null-rd10^{+/+}, Sod2-rd10^{+/+}, or Catalase rd10^{+/+} mice (FIG. 12b). This indicates that coexpression of SOD2 and Catalase, but not expression of either of them alone reduces oxidative damage in cones after rods have degenerated.

### Example 10

### Increased Expression of SOD2 and Catalase in Mitochondria of Photoreceptors Decreases Cone Cell Death in rd10^{+/+} Mice

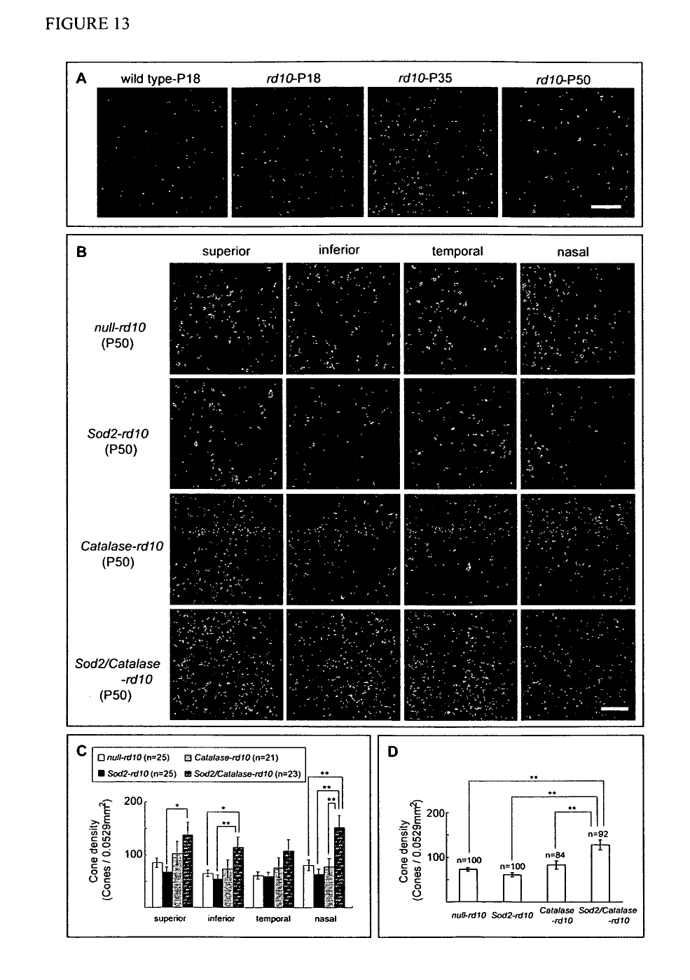

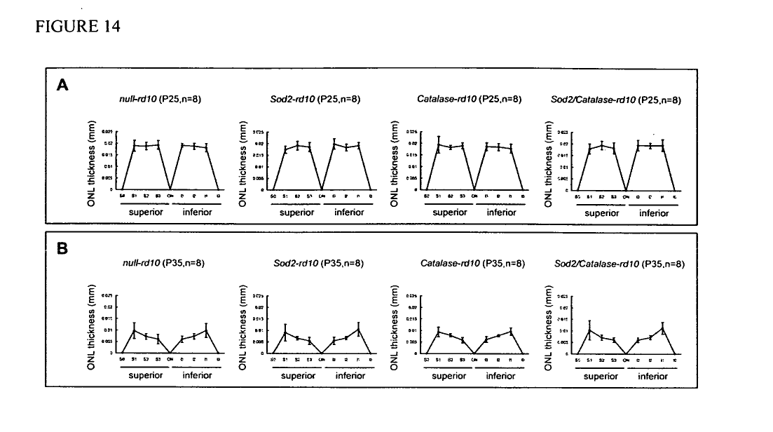

Fluorescence confocal microscopy of peanut agglutinin-stained retinal flat mounts provides a means of assessing cone cell density and, hence, cone survival, provided the same region of the retina is evaluated at different time points. Komeima, 2006. Proc Natil Acad Sci USA 103: 11300-11305). In comparison to P18 wildtype mice, there is no difference in cone density in P18 or P35 rd10 mice (FIG. 13a); however, between P35 and P50, there is substantial loss of cones. This is consistent with observations in multiple models of RP, indicating that cone density is relatively stable until rod degeneration is essentially complete, and then gradual loss of cones occurs (Komeima, 2006. Proc Natil Acad Sci USA 103:11300-11305; Komeima, 2007. J Cell Physiol 213:809-815). However, while the number of cones is similar in P18 and P35 rd10^{+/+} mice, cone morphology is abnormal at P35, because outer segments are missing and inner segments are flattened, indicating that cones are under considerable stress (FIG. 13a). When mice were treated with doxycycline starting at P18, cone density at P50 was significantly greater in Sod2/Catalase-rd10^{+/+} mice compared to null-rd10^{+/+}, Sod2-rd10^{+/+}, or Catalase-rd10^{+/+} (FIG. 13b-d). Cone density was not greater in Sod2-rd10^{+/+} or Catalase-rd10^{+/+} compared to null-rd10^{+/+} mice. This indicates that coexpression of SOD2 and Catalase in the mitochondria of cones, but not either alone, promotes cone survival after rods have degenerated in rd10^{+/+} mice. In contrast to this robust effect on cone survival, coexpression of SOD2 and Catalase, as well as expression of either alone, had no effect on rod survival in rd10^{+/+} mice as demonstrated by failure to prevent thinning of the outer nuclear layer (ONL) at P25 and P35 (FIG. 14).

### Example 11

### Increased Expression of SOD2 and Catalase Preserves Cone Cell Function in P50 rd10^{+/+} Mice

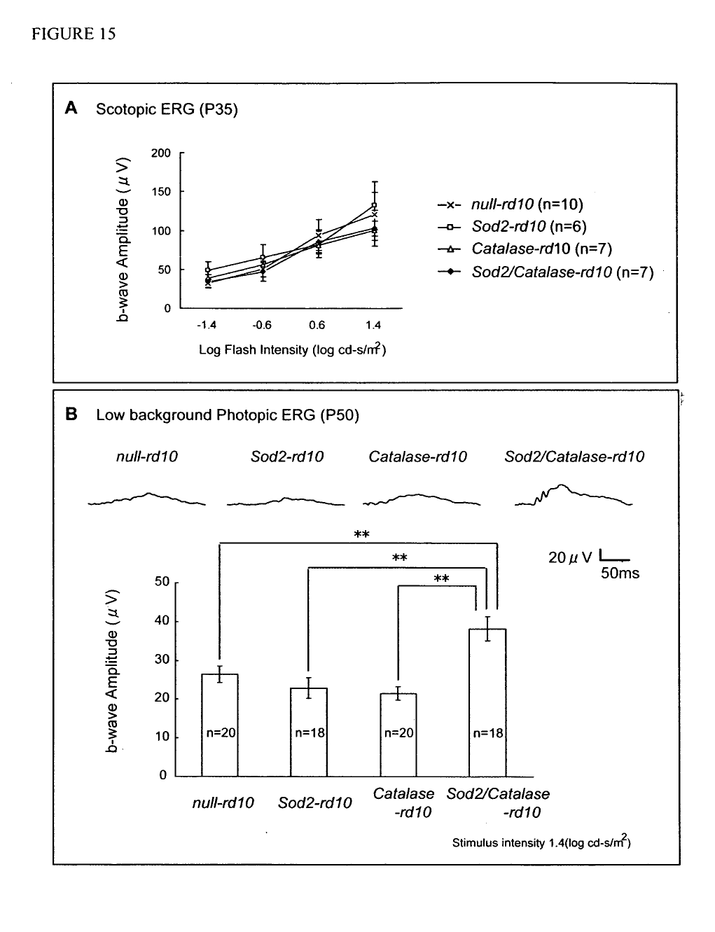

There was no difference in mean scotopic electroretinogram (ERG) b-wave amplitude at P35 in doxycycline-treated nullrd10+/+, Sod2-rd10^{+/+}, Catalase-rd10^{+/+}, and Sod2/Catalaserd10^{+/+} mice, indicating that expression of SOD2 and/or Catalase had no effect on rod function in rd10^{+/+} mice (FIG. 15a). At P50, low background photopic ERGs showed nearly flat waveforms in doxycycline-treated null-rd10^{+/+}, Sod2-rd10^{+/+}, and Catalase-rd10^{+/+} mice, but Sod2/Catalase-rd10^{+/+} mice showed a substantially better waveform and significantly greater mean photopic b-wave amplitude (FIG. 15b). This indicates that coexpression of SOD2 and Catalase in mitochondria of photoreceptors, but not expression of either of them alone, preserves cone cell function after rods have degenerated in rd10^{+/+} mice.

### Example 12

### Deficiency of Superoxide Dismutase 1 (Sod1) Increases Superoxide Radicals and Oxidative Damage in the Retinas of rd10+/+ Mice and Accelerates Loss of Cone Function

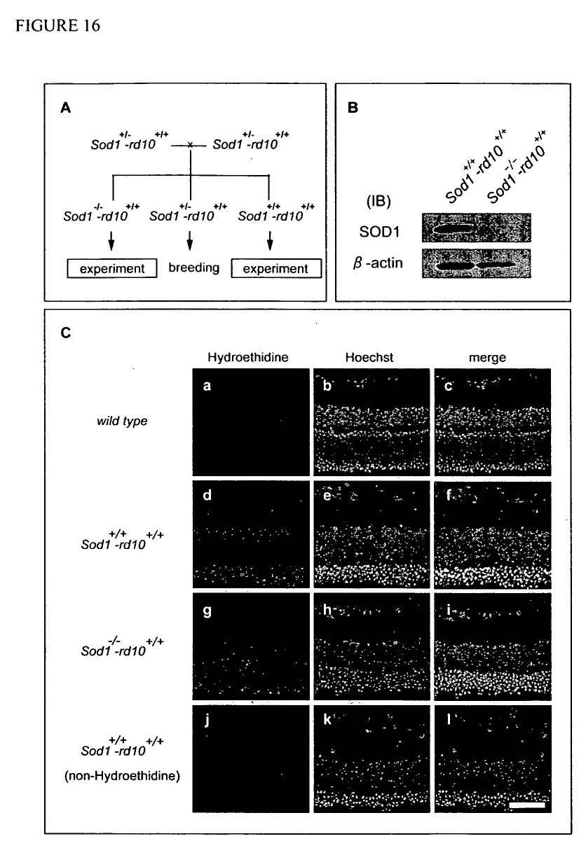

SOD1 is an important component of the antioxidant defense system in the retina because compared to wild type mice, mice deficient in SOD1 are more sensitive to the damaging effects of an intraocular injection of paraquat or exposure to hyperoxia (Dong, 2006). Rd10^{+/+} mice are homozygous for a mutation in rod phosphodiesterase that causes death of rod photoreceptors followed by gradual death of cones from oxidative damage. To determine the effect of deficiency of SOD1 in rd10^{+/+} mice, a mating scheme (FIG. 16A) was devised to generate rd10^{+/+} mice wild type at the Sod1 allele (Sod1^{+/+}-rd10^{+/+} mice), Sod1^{+/-}-rd10^{+/+} mice, and rd10^{+/+} mice deficient in SOD1 (Sod1^{-/-}-rd10^{+/+} mice). Immunoblots confirmed Sod1^{-/-}-rd10^{+/+} mice lacked SOD1 (FIG. 16B).

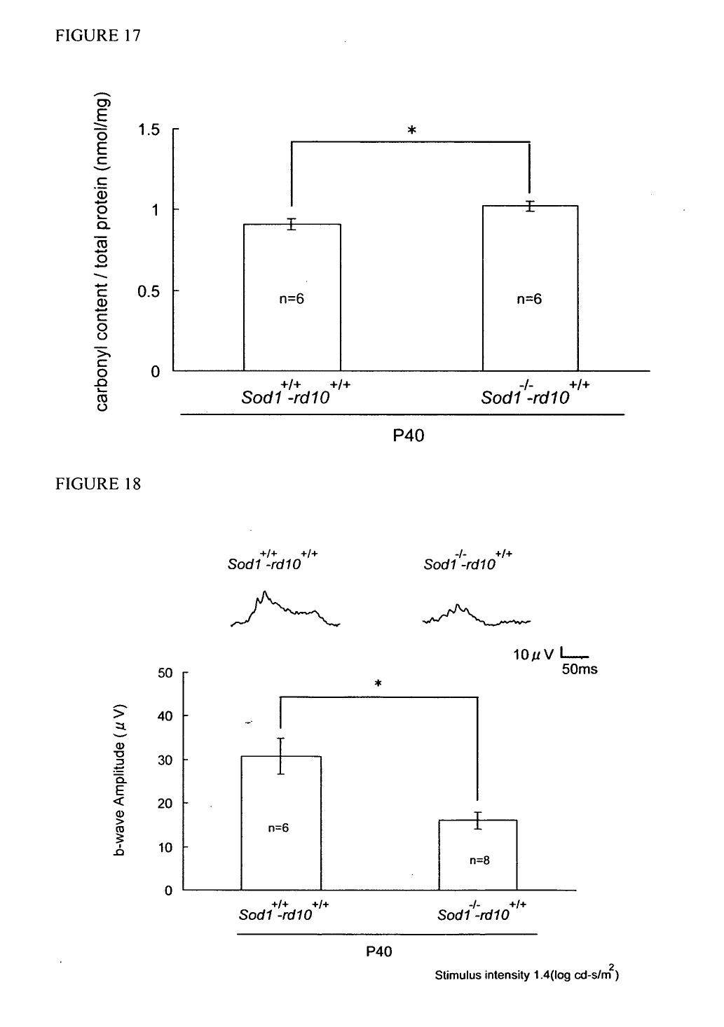

Hydroethidine allows visualization of superoxide radicals because in their presence it is converted to ethidium which binds DNA and fluoresces. Eighteen hours after intravenous injection of hydroethidine, there was minimal fluorescence in the retinas of wild type mice (FIG. 16C, panels a-c), moderate fluorescence primarily in the remaining outer nuclear layer of the retinas of Sod1^{+/+}-rd10^{+/+} mice (FIG. 16C, panels d-f), and strong fluorescence in the retinas of Sod1^{-/-}-rd10^{+/+} mice (FIG. 16C, panels g-I). Without injection of hydroethidine, Sod1^{+/+}-rd10^{+/+} mice showed no fluorescence (FIG. 16C, panels j-l). At P40, levels of carbonyl adducts on proteins were significantly higher in the retinas of Sod1^{-/-}-rd10^{+/+} mice compared to Sod1^{+/+}-rd10^{+/+} mice (FIG. 2). Low background photopic ERGs at P40 substantially better waveforms and significantly higher mean photopic b-wave amplitude for Sod1^{+/+}-rd10^{+/+} mice compared to Sod1^{-/-}-rd10^{+/+} mice (FIG. 17).

### Example 13

### Co-Expression of SOD1 and Cytoplasmic Gpx4 in Photoreceptors Significantly Reduces Retinal Carbonyl Content and Improves Cone Function in rd10^{+/+} Mice

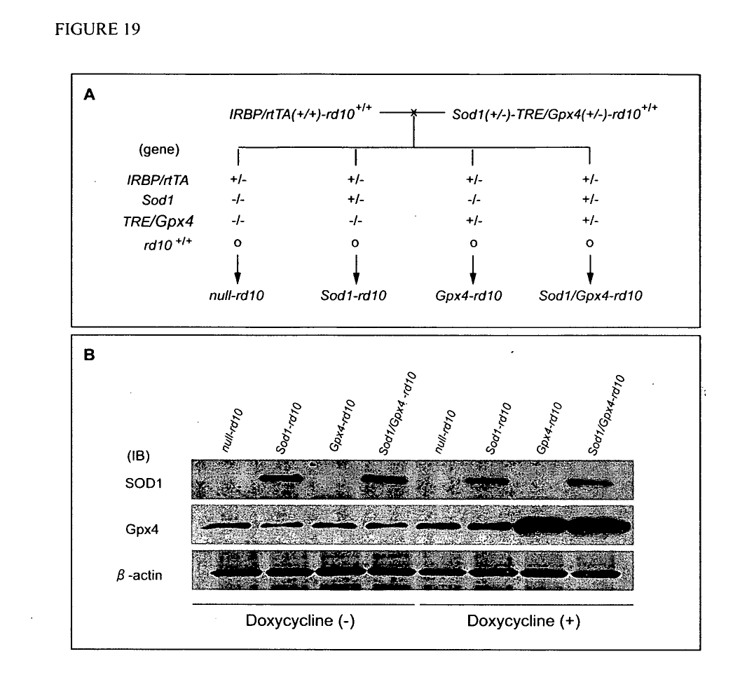

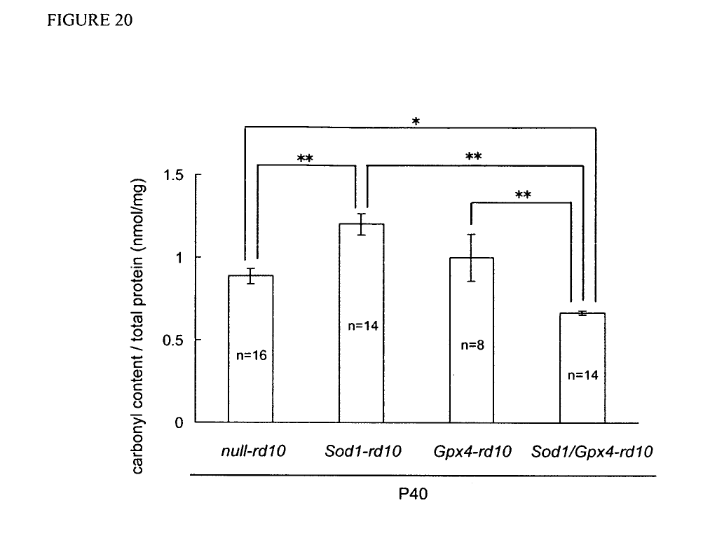

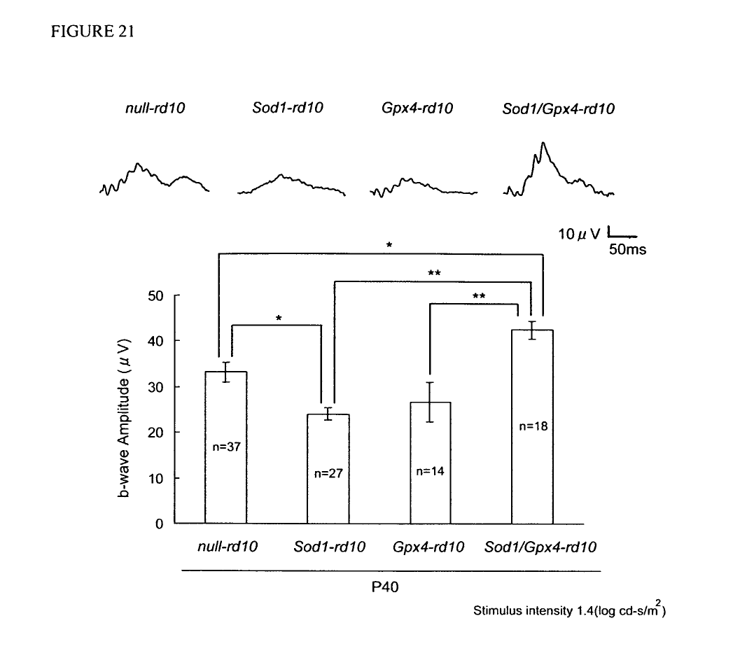

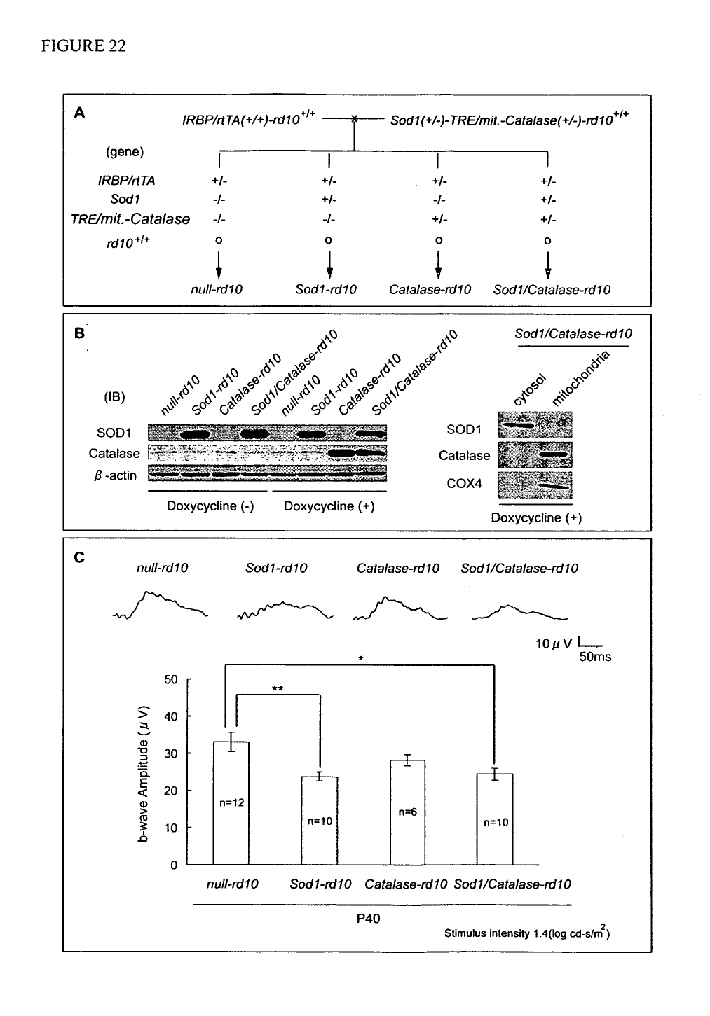

Transgenic mice carrying a β-actin promoter/human Sod1 transgene express high levels of SOD1 in the retina which reduces oxidative damage from intraocular injection of paraquat. Similarly, induced expression of murine cytoplasmic Gpx4 by treatment of IRBP/rtTA-TRE/Gpx4 mice with doxycycline also reduces paraquat-induced oxidative damage in the retina (Lu, 2008). To test the effects of over-expression of SOD1 and Gpx4 on the oxidative damage that occurs in cones of rd10^{+/+} mice, an elaborate crossing scheme was used to generate 4 groups of offspring, null-rd10, Sod1-rd10, Gpx4-rd10, and Sod1/Gpx4-rd10 mice (FIG. 19A). Immunoblots of retinal homogenates showed strong expression of human SOD1 in Sod1-rd10 and Sod1/Gpx4-rd10 mice (FIG. 19B). Background levels of murine Gpx4 were seen in all mice, but when Gpx4-rd10^{+/+} or Sod1/Gpx4-rd10^{+/+} mice were treated with doxycycline, they showed a substantial increase in Gpx4. In doxycycline-treated P40 mice, protein carbonyl content was significantly greater in Sod1-rd10 mice compared to null-rd10 or Sod1/Gpx4-rd10 mice and was significantly less in Sod1/Gpx4-rd10 mice compared to null-rd10, Sod1-rd10 or Gpx4-rd10 mice (FIG. 20). Low background photopic ERGs showed mean photopic b-wave amplitudes that were significantly higher in Sod1/Gpx4-rd10 mice compared to null-rd10, Sod1-rd10, or Gpx4-rd10 mice, and significantly lower in Sod1-rd10 mice than in null-rd10 mice (FIG. 21).

### Example 14

### Co-Expression of SOD1 and Mitochondrial-Targeted Catalase in Photoreceptors does not Preserve Cone Cell Function in rd10^{+/+} Mice

Increased expression of SOD2 increases oxidative stress and promotes cone cell death in rd10^{+/+} mice, but when SOD2 is co-expressed with Catalase that is targeted to mitochondria, cone function is improved compared to rd10^{+/+} mice with wild type levels of SOD2 and Catalase (Usui, 2009. Mol. Ther. 17: 778-786, incorporated herein by reference). We sought to determine if Catalase targeted to mitochondria reversed the damaging effects of over-expression of SOD1. A mating scheme was designed to generate 4 groups of offspring, null-rd10, Sod1-rd10, Catalase-rd10, and Sod1/Catalase-rd10 mice (FIG. 22A). Immunoblots of retinal homogenates showed strong expression of human SOD1 in Sod1-rd10 and Sod1/Catalase-rd10 and strong expression of Catalase in doxycycline-treated Catalase-rd10 and Sod1/Catalase-rd10 mice. Immunoblots of cytosolic and mitochondrial fractions of retinal homogenates showed that only the cytosolic fraction showed a substantial increase in SOD1 and only the mitochondrial fraction showed a substantial increase in Catalase and COX4, which is known to localize to mitochondria (FIG. 22B). Low background photopic ERGs at P40 showed a significant reduction in mean photopic b-wave amplitude in Sod1-rd10 mice and Sod1/Catalase-rd10 mice compared to null-rd10 mice (FIG. 22C).

Polypeptide and nucleic acid sequences referred to herein include the following:

| LOCUS NM_000454 981 bp mRNA linear PRI 21 JUN. 2009 | |
|---|---|
| DEFINITION Homo sapiens superoxide dismutase 1, soluble (SOD1), | |
| mRNA. | |
| MATKAVCVLKGDGPVQGIINFEQKESNGPVKVWGSIKGLTEGLHGFHVHEFGDNTAGCTSAGPHFNPLS | |
| RKHGGPKDEERHVGDLGNVTADKDGVADVSIEDSVISLSGDHCIIGRTLVVHEKADDLGKGGNEESTKT | |
| GNAGSRLACGVIGIAQ | |
| 1 | gtttggggcc agagtgggcg aggcgcggag gtctggccta taaagtagtc gcggagacgg |
| 61 | ggtgctggtt tgcgtcgtag tctcctgcag cgtctggggt ttccgttgca gtcctcggaa |
| 121 | ccaggacctc ggcgtggcct agcgagttat ggcgacgaag gccgtgtgcg tgctgaaggg |
| 181 | cgacggccca gtgcagggca tcatcaattt cgagcagaag gaaagtaatg gaccagtgaa |
| 241 | ggtgtgggga agcattaaag gactgactga aggcctgcat ggattccatg ttcatgagtt |
| 301 | tggagataat acagcaggct gtaccagtgc aggtcctcac tttaatcctc tatccagaaa |
| 361 | acacggtggg ccaaaggatg aagagaggca tgttggagac ttgggcaatg tgactgctga |
| 421 | caaagatggt gtggccgatg tgtctattga agattctgtg atctcactct caggagacca |
| 481 | ttgcatcatt ggccgcacac tggtggtcca tgaaaaagca gatgacttgg gcaaaggtgg |
| 541 | aaatgaagaa agtacaaaga caggaaacgc tggaagtcgt ttggcttgtg gtgtaattgg |
| 601 | gatcgcccaa taaacattcc cttggatgta gtctgaggcc ccttaactca tctgttatcc |
| 661 | tgctagctgt agaaatgtat cctgataaac attaaacact gtaatcttaa aagtgtaatt |
| 721 | gtgtgacttt ttcagagttg ctttaaagta cctgtagtga gaaactgatt tatgatcact |
| 781 | tggaagattt gtatagtttt ataaaactca gttaaaatgt ctgtttcaat gacctgtatt |
| 841 | ttgccagact taaatcacag atgggtatta aacttgtcag aatttctttg tcattcaagc |
| 901 | ctgtgaataa aaaccctgta tggcacttat tatgaggcta ttaaaagaat ccaaattcaa |
| 961 | actaaaaaaa aaaaaaaaaa a |
| LOCUS NM_000636 1593 bp mRNA linear PRI 07 JUN. 2009 | |
| DEFINITION Homo sapiens superoxide dismutase 2, mitochondrial | |
| (SOD2), nuclear gene encoding mitochondrial protein, transcript | |
| variant 1, mRNA. | |
| MLSRAVCGTSRQLAPVLGYLGSRQKHSLPDLPYDYGALEPHINAQIMQLHHSKHHAAYVNNLNVTEEKY | |
| QEALAKGDVTAQIALQPALKFNGGGHINHSIFWTNLSPNGGGEPKGELLEAIKRDFGSFDKFKEKLTAA | |
| SVGVQGSGWGWLGFNKERGHLQIAACPNQDPLQGTTGLIPLLGIDVWEHAYYLQYKNVRPDYLKAIWNV | |
| INWENVTERYMACKK | |
| 1 | gcggtgccct tgcggcgcag ctggggtcgc ggccctgctc cccgcgcttt cttaaggccc |
| 61 | gcgggcggcg caggagcggc actcgtggct gtggtggctt cggcagcggc ttcagcagat |
| 121 | cggcggcatc agcggtagca ccagcactag cagcatgttg agccgggcag tgtgcggcac |
| 181 | cagcaggcag ctggctccgg ttttggggta tctgggctcc aggcagaagc acagcctccc |
| 241 | cgacctgccc tacgactacg gcgccctgga acctcacatc aacgcgcaga tcatgcagct |
| 301 | gcaccacagc aagcaccacg cggcctacgt gaacaacctg aacgtcaccg aggagaagta |
| 361 | ccaggaggcg ttggccaagg gagatgttac agcccagata gctcttcagc ctgcactgaa |
| 421 | gttcaatggt ggtggtcata tcaatcatag cattttctgg acaaacctca gccctaacgg |
| 481 | tggtggagaa cccaaagggg agttgctgga agccatcaaa cgtgactttg gttcctttga |
| 541 | caagtttaag gagaagctga cggctgcatc tgttggtgtc caaggctcag gttggggttg |
| 601 | gcttggtttc aataaggaac ggggacactt acaaattgct gcttgtccaa atcaggatcc |
| 661 | actgcaagga acaacaggcc ttattccact gctggggatt gatgtgtggg agcacgctta |
| 721 | ctaccttcag tataaaaatg tcaggcctga ttatctaaaa gctatttgga atgtaatcaa |
| 781 | ctgggagaat gtaactgaaa gatacatggc ttgcaaaaag taaaccacga tcgttatgct |
| 841 | gagtatgtta agctctttat gactgttttt gtagtggtat agagtactgc agaatacagt |
| 901 | aagctgctct attgtagcat ttcttgatgt tgcttagtca cttatttcat aaacaactta |
| 961 | atgttctgaa taatttctta ctaaacattt tgttattggg caagtgattg aaaatagtaa |
| 1021 | atgctttgtg tgattgaatc tgattggaca ttttcttcag agagctaaat tacaattgtc |
| 1081 | atttataaaa ccatcaaaaa tattccatcc atatactttg gggacttgta gggatgcctt |
| 1141 | tctagtccta ttctattgca gttatagaaa atctagtctt ttgccccagt tacttaaaaa |
| 1201 | taaaatatta acactttccc aagggaaaca ctcggctttc tatagaaaat tgcacttttt |
| 1261 | gtcgagtaat cctctgcagt gatacttctg gtagatgtca cccagtggtt tttgttaggt |
| 1321 | caaatgttcc tgtatagttt ttgcaaatag agctgtatac tgtttaaatg tagcaggtga |
| 1381 | actgaactgg ggtttgctca cctgcacagt aaaggcaaac ttcaacagca aaactgcaaa |
| 1441 | aaggtggttt ttgcagtagg agaaaggagg atgtttattt gcagggcgcc aagcaaggag |
| 1501 | aattgggcag ctcatgcttg agacccaatc tccatgatga cctacaagct agagtattta |
| 1561 | aaggcagtgg taaatttcag gaaagcagaa gtt |
| LOCUS NM_001024465 1035 bp mRNA linear PRI 07 JUN. 2009 | |
| DEFINITION Homo sapiens superoxide dismutase 2, mitochondrial | |
| (SOD2), nucleargene encoding mitochondrial protein, transcript | |
| variant 2, mRNA. | |
| MLSRAVCGTSRQLAPVLGYLGSRQKHSLPDLPYDYGALEPHINAQIMQLHHSKHHAAYVNNLNVTEEKY | |
| QEALAKGDVTAQIALQPALKFNGGGHINHSIFWTNLSPNGGGEPKGELLEAIKRDFGSFDKFKEKLTAA | |
| SVGVQGSGWGWLGFNKERGHLQIAACPNQDPLQGTTGLIPLLGIDVWEHAYYLQYKNVRPDYLKAIWNV | |
| INWENVTERYMACKK | |
| 1 | gcggtgccct tgcggcgcag ctggggtcgc ggccctgctc cccgcgcttt cttaaggccc |
| 61 | gcgggcggcg caggagcggc actcgtggct gtggtggctt cggcagcggc ttcagcagat |
| 121 | cggcggcatc agcggtagca ccagcactag cagcatgttg agccgggcag tgtgcggcac |
| 181 | cagcaggcag ctggctccgg ttttggggta tctgggctcc aggcagaagc acagcctccc |
| 241 | cgacctgccc tacgactacg gcgccctgga acctcacatc aacgcgcaga tcatgcagct |
| 301 | gcaccacagc aagcaccacg cggcctacgt gaacaacctg aacgtcaccg aggagaagta |
| 361 | ccaggaggcg ttggccaagg gagatgttac agcccagata gctcttcagc ctgcactgaa |
| 421 | gttcaatggt ggtggtcata tcaatcatag cattttctgg acaaacctca gccctaacgg |
| 481 | tggtggagaa cccaaagggg agttgctgga agccatcaaa cgtgactttg gttcctttga |
| 541 | caagtttaag gagaagctga cggctgcatc tgttggtgtc caaggctcag gttggggttg |
| 601 | gcttggtttc aataaggaac ggggacactt acaaattgct gcttgtccaa atcaggatcc |
| 661 | actgcaagga acaacaggcc ttattccact gctggggatt gatgtgtggg agcacgctta |
| 721 | ctaccttcag tataaaaatg tcaggcctga ttatctaaaa gctatttgga atgtaatcaa |
| 781 | ctgggagaat gtaactgaaa gatacatggc ttgcaaaaag taaaccacga tcgttatgct |
| 841 | gatcataccc taatgatccc agcaagataa tgtcctgtct tctaagatgt gcatcaagcc |
| 901 | tggtacatac tgaaaaccct ataaggtcct ggataatttt tgtttgatta ttcattgaag |
| 961 | aaacatttat tttccaattg tgtgaagttt ttgactgtta ataaaagaat ctgtcaacca |
| 1021 | tcaaaaaaaa aaaaa |
| LOCUS NM_001024466 918 bp mRNA linear PRI 07 JUN. 2009 | |
| DEFINITION Homo sapiens superoxide dismutase 2, mitochondrial | |
| (SOD2), nuclear gene encoding mitochondrial protein, transcript | |
| variant 3, mRNA. | |
| MLSRAVCGTSRQLAPVLGYLGSRQKHSLPDLPYDYGALEPHINAQIMQLHHSKHHAAYVNNLNVTEEK | |
| YQEALAKGELLEAIKRDFGSFDKFKEKLTAASVGVQGSGWGWLGFNKERGHLQIAACPNQDPLQGTTGL | |
| IPLLGIDVWEHAYYLQYKNVRPDYLKAIWNVINWENVTERYMACKK | |
| 1 | gcggtgccct tgcggcgcag ctggggtcgc ggccctgctc cccgcgcttt cttaaggccc |
| 61 | gcgggcggcg caggagcggc actcgtggct gtggtggctt cggcagcggc ttcagcagat |
| 121 | cggcggcatc agcggtagca ccagcactag cagcatgttg agccgggcag tgtgcggcac |
| 181 | cagcaggcag ctggctccgg ttttggggta tctgggctcc aggcagaagc acagcctccc |
| 241 | cgacctgccc tacgactacg gcgccctgga acctcacatc aacgcgcaga tcatgcagct |
| 301 | gcaccacagc aagcaccacg cggcctacgt gaacaacctg aacgtcaccg aggagaagta |
| 361 | ccaggaggcg ttggccaagg gggagttgct ggaagccatc aaacgtgact ttggttcctt |
| 421 | tgacaagttt aaggagaagc tgacggctgc atctgttggt gtccaaggct caggttgggg |
| 481 | ttggcttggt ttcaataagg aacggggaca cttacaaatt gctgcttgtc caaatcagga |
| 541 | tccactgcaa ggaacaacag gccttattcc actgctgggg attgatgtgt gggagcacgc |
| 601 | ttactacctt cagtataaaa atgtcaggcc tgattatcta aaagctattt ggaatgtaat |
| 661 | caactgggag aatgtaactg aaagatacat ggcttgcaaa aagtaaacca cgatcgttat |
| 721 | gctgatcata ccctaatgat cccagcaaga taatgtcctg tcttctaaga tgtgcatcaa |
| 781 | gcctggtaca tactgaaaac cctataaggt cctggataat ttttgtttga ttattcattg |
| 841 | aagaaacatt tattttccaa ttgtgtgaag tttttgactg ttaataaaag aatctgtcaa |
| 901 | ccatcaaaaa aaaaaaaa |
| LOCUS NM_003102 PRI 24 MAY 2009 | |
| DEFINITION Homo sapiens superoxide dismutase 3, extracellular (SOD3) | |
| MLALLCSCLLLAAGASDAWTGEDSAEPNSDSAEWIRDMYAKVTEIWQEVMQRRDDDGALHAACQVQPSA | |
| TLDAAQPRVTGVVLFRQLAPRAKLDAFFALEGFPTEPNSSSRAIHVHQFGDLSQGCESTGPHYNPLAVP | |
| HPQHPGDFGNFAVRDGSLWRYRAGLAASLAGPHSIVGRAVVVHAGEDDLGRGGNQASVENGNAGRRLAC | |
| CVVGVCGPGLWERQAREHSERKKRRRESECKAA | |
| LOCUS NM_001752 PRI 24 MAY 2009 | |
| DEFINITION Homo sapiens catalase (CAT), mRNA. | |
| MADSRDPASDQMQHWKEQRAAQKADVLTTGAGNPVGDKLNVITVGPRGPLLVQDVVFTDEMAHFDRERI | |
| PERVVHAKGAGAFGYFEVTHDITKYSKAKVFEHIGKKTPIAVRFSTVAGESGSADTVRDPRGFAVKFYT | |
| EDGNWDLVGNNTPIFFIRDPILFPSFIHSQKRNPQTHLKDPDMVWDFWSLRPESLHQVSFLFSDRGIPD | |
| GHRHMNGYGSHTFKLVNANGEAVYCKFHYKTDQGIKNLSVEDAARLSQEDPDYGIRDLFNAIATGKYPS | |
| WTFYIQVMTFNQAETFPFNPFDLTKVWPHKDYPLIPVGKLVLNRNPVNYFAEVEQIAFDPSNMPPGIEA | |
| SPDKMLQGRLFAYPDTHRHRLGPNYLHIPVNCPYRARVANYQRDGPMCMQDNQGGAPNYYPNSFGAPEQ | |
| QPSALEHSIQYSGEVRRFNTANDDNVTQVRAFYVNVLNEEQRKRLCENIAGHLKDAQIFIQKKAVKNFT | |
| EVHPDYGSHIQALLDKYNAEKPKNAIHTFVQSGSHLAAREKANL | |
| 1 | ggcaacaggc agatttgcct gctgagggtg gagacccacg agccgaggcc tcctgcagtg |
| 61 | ttctgcacag caaaccgcac gctatggctg acagccggga tcccgccagc gaccagatgc |
| 121 | agcactggaa ggagcagcgg gccgcgcaga aagctgatgt cctgaccact ggagctggta |
| 181 | acccagtagg agacaaactt aatgttatta cagtagggcc ccgtgggccc cttcttgttc |
| 241 | aggatgtggt tttcactgat gaaatggctc attttgaccg agagagaatt cctgagagag |
| 301 | ttgtgcatgc taaaggagca ggggcctttg gctactttga ggtcacacat gacattacca |
| 361 | aatactccaa ggcaaaggta tttgagcata ttggaaagaa gactcccatc gcagttcggt |
| 421 | tctccactgt tgctggagaa tcgggttcag ctgacacagt tcgggaccct cgtgggtttg |
| 481 | cagtgaaatt ttacacagaa gatggtaact gggatctcgt tggaaataac acccccattt |
| 541 | tcttcatcag ggatcccata ttgtttccat cttttatcca cagccaaaag agaaatcctc |
| 601 | agacacatct gaaggatccg gacatggtct gggacttctg gagcctacgt cctgagtctc |
| 661 | tgcatcaggt ttctttcttg ttcagtgatc gggggattcc agatggacat cgccacatga |
| 721 | atggatatgg atcacatact ttcaagctgg ttaatgcaaa tggggaggca gtttattgca |
| 781 | aattccatta taagactgac cagggcatca aaaacctttc tgttgaagat gcggcgagac |
| 841 | tttcccagga agatcctgac tatggcatcc gggatctttt taacgccatt gccacaggaa |
| 901 | agtacccctc ctggactttt tacatccagg tcatgacatt taatcaggca gaaacttttc |
| 961 | catttaatcc attcgatctc accaaggttt ggcctcacaa ggactaccct ctcatcccag |
| 1021 | ttggtaaact ggtcttaaac cggaatccag ttaattactt tgctgaggtt gaacagatag |
| 1081 | ccttcgaccc aagcaacatg ccacctggca ttgaggccag tcctgacaaa atgcttcagg |
| 1141 | gccgcctttt tgcctatcct gacactcacc gccatcgcct gggacccaat tatcttcata |
| 1201 | tacctgtgaa ctgtccctac cgtgctcgag tggccaacta ccagcgtgac ggcccgatgt |
| 1261 | gcatgcagga caatcagggt ggtgctccaa attactaccc caacagcttt ggtgctccgg |
| 1321 | aacaacagcc ttctgccctg gagcacagca tccaatattc tggagaagtg cggagattca |
| 1381 | acactgccaa tgatgataac gttactcagg tgcgggcatt ctatgtgaac gtgctgaatg |
| 1441 | aggaacagag gaaacgtctg tgtgagaaca ttgccggcca cctgaaggat gcacaaattt |
| 1501 | tcatccagaa gaaagcggtc aagaacttca ctgaggtcca ccctgactac gggagccaca |
| 1561 | tccaggctct tctggacaag tacaatgctg agaagcctaa gaatgcgatt cacacctttg |
| 1621 | tgcagtccgg atctcacttg gcggcaaggg agaaggcaaa tctgtgaggc cggggccctg |
| 1681 | cacctgtgca gcgaagctta gcgttcatcc gtgtaacccg ctcatcactg gatgaagatt |
| 1741 | ctcctgtgct agatgtgcaa atgcaagcta gtggcttcaa aatagagaat cccactttct |
| 1801 | atagcagatt gtgtaacaat tttaatgcta tttccccagg ggaaaatgaa ggttaggatt |
| 1861 | taacagtcat ttaaaaaaaa aatttgtttt gacggatgat tggattattc atttaaaatg |
| 1921 | attagaaggc aagtttctag ctagaaatat gattttattt gacaaaattt gttgaaatta |
| 1981 | tgtatgttta catatcacct catggcctat tatattaaaa tatggctata aatatataaa |
| 2041 | aagaaaagat aaagatgatc tactcagaaa tttttatttt tctaaggttc tcataggaaa |
| 2101 | agtacattta atacagcagt gtcatcagaa gataacttga gcaccgtcat ggcttaatgt |
| 2161 | ttattcctga taataattga tcaaattcat ttttttcact ggagttacat taatgttaat |
| 2221 | tcagcactga tttcacaaca gatcaatttg taattgctta catttttaca ataaataatc |
| 2281 | tgtacgtaag aacaaaaaaa aaaaa |
| LOCUS NM_000581 921 bp mRNA linear PRI 21 JUN. 2009 | |
| DEFINITION Homo sapiens glutathione peroxidase 1 (GPX1), transcript | |
| variant 1, mRNA. | |
| MCAARLAAAAAAAQSVYAFSARPLAGGEPVSLGSLRGKVLLIENVASLUGTTVRDYTQMNELQRRLGPR | |
| GLVVLGFPCNQFGHQENAKNEEILNSLKYVRPGGGFEPNFMLFEKCEVNGAGAHPLFAFLREALPAPSD | |
| DATALMTDPKLITWSPVCRNDVAWNFEKFLVGPDGVPLRRYSRRFQTIDIEPDIEALLSQGPSCA | |
| 1 | cagttaaaag gaggcgcctg ctggcctccc cttacagtgc ttgttcgggg cgctccgctg |
| 61 | gcttcttgga caattgcgcc atgtgtgctg ctcggctagc ggcggcggcg gcggcggccc |
| 121 | agtcggtgta tgccttctcg gcgcgcccgc tggccggcgg ggagcctgtg agcctgggct |
| 181 | ccctgcgggg caaggtacta cttatcgaga atgtggcgtc cctctgaggc accacggtcc |
| 241 | gggactacac ccagatgaac gagctgcagc ggcgcctcgg accccggggc ctggtggtgc |
| 301 | tcggcttccc gtgcaaccag tttgggcatc aggagaacgc caagaacgaa gagattctga |
| 361 | attccctcaa gtacgtccgg cctggtggtg ggttcgagcc caacttcatg ctcttcgaga |
| 421 | agtgcgaggt gaacggtgcg ggggcgcacc ctctcttcgc cttcctgcgg gaggccctgc |
| 481 | cagctcccag cgacgacgcc accgcgctta tgaccgaccc caagctcatc acctggtctc |
| 541 | cggtgtgtcg caacgatgtt gcctggaact ttgagaagtt cctggtgggc cctgacggtg |
| 601 | tgcccctacg caggtacagc cgccgcttcc agaccattga catcgagcct gacatcgaag |
| 661 | ccctgctgtc tcaagggccc agctgtgcct agggcgcccc tcctaccccg gctgcttggc |
| 721 | agttgcagtg ctgctgtctc gggggggttt tcatctatga gggtgtttcc tctaaaccta |
| 781 | cgagggagga acacctgatc ttacagaaaa taccacctcg agatgggtgc tggtcctgtt |
| 841 | gatcccagtc tctgccagac caaggcgagt ttccccacta ataaagtgcc gggtgtcagc |
| 901 | agaaaaaaaa aaaaaaaaaa a |
| LOCUS NM_201397 1200 bp mRNA linear PRI 21 JUN. 2009 | |
| DEFINITION Homo sapiens glutathione peroxidase 1 (GPX1), transcript | |
| variant 2, mRNA. | |
| MCAARLAAAAAAAQSVYAFSARPLAGGEPVSLGSLRGKVLLIENVASLUGTTVRDYTQMNELQRRLGPR | |
| GLVVLGFPCNQFGHQVRRAERGGAGADVQ | |
| 1 | cagttaaaag gaggcgcctg ctggcctccc cttacagtgc ttgttcgggg cgctccgctg |
| 61 | gcttcttgga caattgcgcc atgtgtgctg ctcggctagc ggcggcggcg gcggcggccc |
| 121 | agtcggtgta tgccttctcg gcgcgcccgc tggccggcgg ggagcctgtg agcctgggct |
| 181 | ccctgcgggg caaggtacta cttatcgaga atgtggcgtc cctctgaggc accacggtcc |
| 241 | gggactacac ccagatgaac gagctgcagc ggcgcctcgg accccggggc ctggtggtgc |
| 301 | tcggcttccc gtgcaaccag tttgggcatc aggtgcgccg ggcggagcgg ggcggggcgg |
| 361 | gggcggacgt gcagtagtgg ctgggggcgc cggcggtgtg ctggtgggtg ccgtcggctc |
| 421 | catgcgcgga gagtctggct actctctcgt ttcctttctg ttgctcgtag ctgctgaaat |
| 481 | tcctctccgc ccttgggatt gcgcatggag ggcaaaatcc cggtgactca tagaaaatct |
| 541 | cccttgtttg tggttagaac gtttctctcc tcctcttgac cccgggttct agctgccctt |
| 601 | ctctcctgta ggagaacgcc aagaacgaag agattctgaa ttccctcaag tacgtccggc |
| 661 | ctggtggtgg gttcgagccc aacttcatgc tcttcgagaa gtgcgaggtg aacggtgcgg |
| 721 | gggcgcaccc tctcttcgcc ttcctgcggg aggccctgcc agctcccagc gacgacgcca |
| 781 | ccgcgcttat gaccgacccc aagctcatca cctggtctcc ggtgtgtcgc aacgatgttg |
| 841 | cctggaactt tgagaagttc ctggtgggcc ctgacggtgt gcccctacgc aggtacagcc |
| 901 | gccgcttcca gaccattgac atcgagcctg acatcgaagc cctgctgtct caagggccca |
| 961 | gctgtgccta gggcgcccct cctaccccgg ctgcttggca gttgcagtgc tgctgtctcg |
| 1021 | ggggggtttt catctatgag ggtgtttcct ctaaacctac gagggaggaa cacctgatct |
| 1081 | tacagaaaat accacctcga gatgggtgct ggtcctgttg atcccagtct ctgccagacc |
| 1141 | aaggcgagtt tccccactaa taaagtgccg ggtgtcagca gaaaaaaaaa aaaaaaaaaa |
| LOCUS NM_002085 PRI 24 MAY 2009 | |
| DEFINITION Homo sapiens glutathione peroxidase 4 (phospholipid | |
| hydroperoxidase) (GPX4), transcript variant 1, mRNA. | |
| MSLGRLCRLLKPALLCGALAAPGLAGTMCASRDDWRCARSMHEFSAKDIDGHMVNLDKYRGFVCIVTNV | |
| ASQUGKTEVNYTQLVDLHARYAECGLRILAFPCNQFGKQEPGSNEEIKEFAAGYNVKFDMFSKICVNGD | |
| DAHPLWKWMKIQPKGKGILGNAIKWNFTKFLIDKNGCVVKRYGPMEEPLVIEKDLPHYF | |
| 1 | gagcgctctg gagggcgtgg ccgtgggaaa ggaggcgcgg aaagccgacg cgcgtccatt |
| 61 | ggtcggctgg acgaggggag gagccgctgg ctcccagccc cgccgcgatg agcctcggcc |
| 121 | gcctttgccg cctactgaag ccggcgctgc tctgtggggc tctggccgcg cctggcctgg |
| 181 | ccgggaccat gtgcgcgtcc cgggacgact ggcgctgtgc gcgctccatg cacgagtttt |
| 241 | ccgccaagga catcgacggg cacatggtta acctggacaa gtaccggggc ttcgtgtgca |
| 301 | tcgtcaccaa cgtggcctcc cagtgaggca agaccgaagt aaactacact cagctcgtcg |
| 361 | acctgcacgc ccgatacgct gagtgtggtt tgcggatcct ggccttcccg tgtaaccagt |
| 421 | tcgggaagca ggagccaggg agtaacgaag agatcaaaga gttcgccgcg ggctacaacg |
| 481 | tcaaattcga tatgttcagc aagatctgcg tgaacgggga cgacgcccac ccgctgtgga |
| 541 | agtggatgaa gatccaaccc aagggcaagg gcatcctggg aaatgccatc aagtggaact |
| 601 | tcaccaagtt cctcatcgac aagaacggct gcgtggtgaa gcgctacgga cccatggagg |
| 661 | agcccctggt gatagagaag gacctgcccc actatttcta gctccacaag tgtgtggccc |
| 721 | cgcccgagcc cctgcccacg cccttggagc cttccaccgg cactcatgac ggcctgcctg |
| 781 | caaacctgct ggtggggcag acccgaaaat ccagcgtgca ccccgccgga ggaaggtccc |
| 841 | atggcctgct gggcttggct cggcgccccc acccctggct accttgtggg aataaacaga |
| 901 | caaattagcc tgctggaaaa aaaaaaaaaa aaaaaaaaaa aa |
| LOCUS NM_002083 PRI 15 FEB. 2009 | |
| DEFINITION Homo sapiens glutathione peroxidase 2 (gastrointestinal) | |
| (GPX2) | |
| MAFIAKSFYDLSAISLDGEKVDFNTFRGRAVLIENVASLUGTTTRDFTQLNELQCRFPRRLVVLGFPCN | |
| QFGHQENCQNEEILNSLKYVRPGGGYQPTFTLVQKCEVNGQNEHPVFAYLKDKLPYPYDDPFSLMTDPK | |
| LIIWSPVRRSDVAWNFEKFLIGPEGEPFRRYSRTFPTINIEPDIKRLLKVAI | |
| LOCUS NM_002084 PRI 10 MAY 2009 | |
| DEFINITION Homo sapiens glutathione peroxidase 3 (plasma) (GPX3) | |
| MARLLQASCLLSLLLAGFVSQSRGQEKSKMDCHGGISGTIYEYGALTIDGEEYIPFKQYAGKYVLFVNV | |
| ASYUGLTGQYIELNALQEELAPFGLVILGFPCNQFGKQEPGENSEILPTLKYVRPGGGFVPNFQLFEKG | |
| DVNGEKEQKFYTFLKNSCPPTSELLGTSDRLFWEPMKVHDIRWNFEKFLVGPDGIPIMRWHHRTTVSNV | |
| KMDILSYMRRQAALGVKRK | |
| LOCUS NM_001509 PRI 02 NOV. 2008 | |
| DEFINITION Homo sapiens glutathione peroxidase 5 (epididymal | |
| androgen-relatedprotein) (GPX5), transcript variant 1 | |
| MTTQLRVVHLLPLLLACFVQTSPKQEKMKMDCHKDEKGTIYDYEAIALNKNEYVSFKQYVGKHILFVNV | |
| ATYCGLTAQYPELNALQEELKPYGLVVLGFPCNQFGKQEPGDNKEILPGLKYVRPGGGFVPSFQLFEKG | |
| DVNGEKEQKVFSFLKHSCPHPSEILGTFKSISWDPVKVHDIRWNFEKFLVGPDGIPVMRWSHRATVSSV | |
| KTDILAYLKQFKTK | |
| LOCUS DQ088982 PRI 18 JUN. 2005 | |
| DEFINITION Homo sapiens glutathione peroxidase 6 (olfactory) (GPX6) | |
| MFQQFQASCLVLLFLVGFAQQTLKPQNRKVDCNKGVTGTIYEYGALTLNGEEYIQFKQFAGKHVLFVNV | |
| AAYUGLAAQYPELNALQEELKNFGVIVLAFPCNQFGKQEPGTNSEILLGLKYVCPGSGFVPSFQLFEKG | |
| DVNGEKEQKVFTFLKNSCPPTSDLLGSSSQLFWEPMKVHDIRWNFEKFLVGPDGVPVMHWFHQAPVSTV | |
| KSDILEYLKQFNTH | |
| LOCUS NM_015696 PRI 24 OCT. 2008 | |
| DEFINITION Homo sapiens glutathione peroxidase 7 (GPX7) | |
| MVAATVAAAWLLLWAAACAQQEQDFYDFKAVNIRGKLVSLEKYRGSVSLVVNVASECGFTDQHYRALQQ | |
| LQRDLGPHHFNVLAFPCNQFGQQEPDSNKEIESFARRTYSVSFPMFSKIAVTGTGAHPAFKYLAQTSGK | |
| EPTWNFWKYLVAPDGKVVGAWDPTVSVEEVRPQITALVRKLILLKREDL | |
| LOCUS NM_001008397 PRI 22 OCT. 2008 | |
| DEFINITION Homo sapiens glutathione peroxidase 8 (putative) (GPX8) | |
| MEPLAAYPLKCSGPRAKVFAVLLSIVLCTVTLFLLQLKFLKPKINSFYAFEVKDAKGRTVSLEKYKGKV | |
| SLVVNVASDCQLTDRNYLGLKELHKEFGPSHFSVLAFPCNQFGESEPRPSKEVESFARKNYGVTFPIFH | |
| KIKILGSEGEPAFRFLVDSSKKEPRWNFWKYLVNPEGQVVKFWKPEEPIEVIRPDIAALVRQVIIKKKE | |
| DL | |
| Chickent β globin promoter (CBP) | |
| 1 | actagttatt aatagtaatc aattacgggg tcattagttc atagcccata tatggagttc |
| 61 | cgcgttacat aacttacggt aaatggcccg cctggctgac cgcccaacga cccccgccca |
| 121 | ttgacgtcaa taatgacgta tgttcccata gtaacgccaa tagggacttt ccattgacgt |
| 181 | caatgggtgg agtatttacg gtaaactgcc cacttggcag tacatcaagt gtatcatatg |
| 241 | ccaagtacgc cccctattga cgtcaatgac ggtaaatggc ccgcctggca ttatgcccag |
| 301 | tacatgacct tatgggactt tcctacttgg cagtacatct acgtattagt catcgctatt |
| 361 | accatggtcg aggtgagccc cacgttctgc ttcactctcc ccatctcccc cccctcccca |
| 421 | cccccaattt tgtatttatt tattttttaa ttattttgtg cagcgatggg ggcggggggg |
| 481 | gggggggggc gcgcgccagg cggggcgggg cggggcgagg ggcggggcgg ggcgaggcgg |
| 541 | agaggtgcgg cggcagccaa tcagagcggc gcgctccgaa agtttccttt tatggcgagg |
| 601 | cggcggcggc ggcggcccta taaaaagcga agcgcgcggc gggcggggag tcgctgcgac |
| 661 | gctgccttcg ccccgtgccc cgctccgccg ccgcctcgcg ccgcccgccc cggctctgac |
| 721 | tgaccgcgtt actcccacag gtgagcgggc gggacggccc ttctcctccg ggctgtaatt |
| 781 | agcgcttggt ttaatgacgg cttgtttctt ttctgtggct gcgtgaaagc cttgaggggc |
| 841 | tccgggaggg ccctttgtgc ggggggagcg gctcgggggg tgcgtgcgtg tgtgtgtgcg |
| 901 | tggggagcgc cgcgtgcggc tccgcgctgc ccggcggctg tgagcgctgc gggcgcggcg |
| 961 | cggggctttg tgcgctccgc agtgtgcgcg aggggagcgc ggccgggggc ggtgccccgc |
| 1021 | ggtgcggggg gggctgcgag gggaacaaag gctgcgtgcg gggtgtgtgc gtgggggggt |
| 1081 | gagcaggggg tgtgggcgcg tcggtcgggc tgcaaccccc cctgcacccc cctccccgag |
| 1141 | ttgctgagca cggcccggct tcgggtgcgg ggctccgtac ggggcgtggc gcggggctcg |
| 1201 | ccgtgccggg cggggggtgg cggcaggtgg gggtgccggg cggggcgggg ccgcctcggg |
| 1261 | ccggggaggg ctcgggggag gggcgcggcg gcccccggag cgccggcggc tgtcgaggcg |
| 1321 | cggcgagccg cagccattgc cttttatggt aatcgtgcga gagggcgcag ggacttcctt |
| 1381 | tgtcccaaat ctgtgcggag ccgaaatctg ggaggcgccg ccgcaccccc tctagcgggc |
| 1441 | gcggggcgaa gcggtgcggc gccggcagga aggaaatggg cggggagggc cttcgtgcgt |
| 1501 | cgccgcgccg ccgtcccctt ctccctctcc agcctcgggg ctgtccgcgg ggggacggct |
| 1561 | gccttcgggg gggacggggc agggcggggt tcggcttctg gcgtgtgacc ggcggctcta |
| 1621 | gagcctctgc taaccatgtt catgccttct tctttttcct acagctcctg ggcaacgtgc |
| 1681 | tggttattgt gctgtctcat cattttggca aagaattcgg cttgatcgaa gcttgcccac |
| 1741 | c |
| Small (sm)CBA promoter | |
| 1 | aattcggtac cctagttatt aatagtaatc aattacgggg tcattagttc atagcccata |
| 61 | tatggagttc cgcgttacat aacttacggt aaatggcccg cctggctgac cgcccaacga |
| 121 | cccccgccca ttgacgtcaa taatgacgta tgttcccata gtaacgccaa tagggacttt |
| 181 | ccattgacgt caatgggtgg actatttacg gtaaactgcc cacttggcag tacatcaagt |
| 241 | gtatcatatg ccaagtacgc cccctattga cgtcaatgac ggtaaatggc ccgcctggca |
| 301 | ttatgcccag tacatgacct tatgggactt tcctacttgg cagtacatct acgtattagt |
| 361 | catcgctatt accatggtcg aggtgagccc cacgttctgc ttcactctcc ccatctcccc |
| 481 | cccctcccca cccccaattt tgtatttatt tattttttaa ttattttgtg cagcgatggg |
| 541 | ggcggggggg gggggggggc gcgcgccagg cggggcgggg cggggcgagg ggcggggcgg |
| 601 | ggcgaggcgg agaggtgcgg cggcagccaa tcagagcggc gcgctccgaa agtttccttt |
| 661 | tatggcgagg cggcggcggc ggcggcccta taaaaagcga agcgcgcggc gggcgggagt |
| 721 | cgctgcgacg ctgccttcgc cccgtgcccc gctccgccgc cgcctcgcgc cgcccgcccc |
| 781 | ggctctgact gaccgcgtta ctcccacagg tgagcgggcg ggacggccct tctcctccgg |
| 841 | gctgtaatta gcgcttggtt taatgacggc ttgtttcttt tctgtggctg cgtgaaagcc |
| 901 | ttgaggggct ccgggagcta gagcctctgc taaccatgtt catgccttct tctttttcct |
| 953 | acagctcctg ggcaacgtgc tggttattgt gctgtctcat cattttggca aag |

LOCUS NM_001145453 PRI 26 APR. 2009
DEFINITION Homo sapiens GDNF family receptor alpha 1 (GFRA1),
transcript variant 3
MFLATLYFALPLLDLLLSAEVSGGDRLDCVKASDQCLKEQSCSTKYRTLRQCVAGKETNFSLASGLEAK
DECRSAMEALKQKSLYNCRCKRGMKKEKNCLRIYWSMYQSLQGNDLLEDSPYEPVNSRLSDIFRVVPFI
SVEHIPKGNNCLDAAKACNLDDICKKYRSAYITPCTTSVSNDVCNRRKCHKALRQFFDKVPAKHSYGML
FCSCRDIACTERRRQTIVPVCSYEEREKPNCLNLQDSCKTNYICRSRLADFFTNCQPESRSVSSCLKEN
YADCLLAYSGLIGTVMTPNYIDSSSLSVAPWCDCSNSGNDLEECLKFLNFFKDNTCLKNAIQAFGNGSD
VTVWQPAFPVQTTTATTTTALRVKNKPLGPAGSENEIPTHVLPPCANLQAQKLKSNVSGNTHLCISNGN
YEKEGLGASSHITTKSMAAPPSCGLSPLLVLVVTALSTLLSLTETS
LOCUS NM_145793 PRI 26 APR. 2009
DEFINITION Homo sapiens GDNF family receptor alpha 1 (GFRA1),
transcript variant 2
MFLATLYFALPLLDLLLSAEVSGGDRLDCVKASDQCLKEQSCSTKYRTLRQCVAGKETNFSLASGLEAK
DECRSAMEALKQKSLYNCRCKRGMKKEKNCLRIYWSMYQSLQGNDLLEDSPYEPVNSRLSDIFRVVPFI
SVEHIPKGNNCLDAAKACNLDDICKKYRSAYITPCTTSVSNDVCNRRKCHKALRQFFDKVPAKHSYGML
FCSCRDIACTERRRQTIVPVCSYEEREKPNCLNLQDSCKTNYICRSRLADFFTNCQPESRSVSSCLKEN
YADCLLAYSGLIGTVMTPNYIDSSSLSVAPWCDCSNSGNDLEECLKFLNFFKDNTCLKNAIQAFGNGSD
VTVWQPAFPVQTTTATTTTALRVKNKPLGPAGSENEIPTHVLPPCANLQAQKLKSNVSGNTHLCISNGN
YEKEGLGASSHITTKSMAAPPSCGLSPLLVLVVTALSTLLSLTETS
LOCUS NM_005264 PRI 26 APR. 2009
DEFINITION Homo sapiens GDNF family receptor alpha 1 (GFRA1),
transcript variant 1
MFLATLYFALPLLDLLLSAEVSGGDRLDCVKASDQCLKEQSCSTKYRTLRQCVAGKETNFSLASGLEAK
DECRSAMEALKQKSLYNCRCKRGMKKEKNCLRIYWSMYQSLQGNDLLEDSPYEPVNSRLSDIFRVVPFI
SDVFQQVEHIPKGNNCLDAAKACNLDDICKKYRSAYITPCTTSVSNDVCNRRKCHKALRQFFDKVPAKH
SYGMLFCSCRDIACTERRRQTIVPVCSYEEREKPNCLNLQDSCKTNYICRSRLADFFTNCQPESRSVSS
CLKENYADCLLAYSGLIGTVMTPNYIDSSSLSVAPWCDCSNSGNDLEECLKFLNFFKDNTCLKNAIQAF
GNGSDVTVWQPAFPVQTTTATTTTALRVKNKPLGPAGSENEIPTHVLPPCANLQAQKLKSNVSGNTHLC
ISNGNYEKEGLGASSHITTKSMAAPPSCGLSPLLVLVVTALSTLLSLTETS
LOCUS NM_199234 PRI 26 APR. 2009
DEFINITION Homo sapiens glial cell derived neurotrophic factor
(GDNF), transcript variant 3
MGCRGCLPGAAPHRVRLPAANPENSRGKGRRGQRGKNRGCVLTAIHLNVTDLGLGYETKEELIFRYCSG
SCDAAETTYDKILKNLSRNRRLVSDKVGQACCRPIAFDDDLSFLDDNLVYHILRKHSAKRCGCI
LOCUS NM_199231 PRI 26 APR. 2009
DEFINITION Homo sapiens glial cell derived neurotrophic factor
(GDNF), transcript variant 2
MKLWDVVAVCLVLLHTASAFPLPAANMPEDYPDQFDDVMDFIQATIKRLKRSPDKQMAVLPRRERNRQA
AAANPENSRGKGRRGQRGKNRGCVLTAIHLNVTDLGLGYETKEELIFRYCSGSCDAAETTYDKILKNLS
RNRRLVSDKVGQACCRPIAFDDDLSFLDDNLINHILRKHSAKRCGCI
LOCUS NM_000514 PRI 26 APR. 2009
DEFINITION Homo sapiens glial cell derived neurotrophic factor
(GDNF), transcript variant 1
MKLWDVVAVCLVLLHTASAFPLPAGKRPPEAPAEDRSLGRRRAPFALSSDSNMPEDYPDQFDDVMDFIQ
ATIKRLKRSPDKQMAVLPRRERNRQAAAANPENSRGKGRRGQRGKNRGCVLTAIHLNVTDLGLGYETKE
ELIFRYCSGSCDAAETTYDKILKNLERNRRLVSDKVGQACCRPIAFDDDLSFLDDNLVYHILRKHSAKR
CGCI

Nucleic acids encoding the various polypeptide sequences can readily be determined by one of skill in the art, and any sequence encoding any of the polypeptide sequences of the invention falls within the scope of the invention.

All patents, patent applications, GenBank numbers in the version available as of the priority date of the instant application, and published references cited herein are hereby incorporated by reference in their entirety as if they were incorporated individually. While this invention has been particularly shown and described with references to preferred embodiments thereof, it will be understood by those skilled in the art that various changes in form and details may be made therein without departing from the scope of the invention encompassed by the appended claims.

1. A method for the prevention, amelioration, or treatment of a disease or condition associated with oxidative stress in a subject comprising administration of a therapeutically effective amount of a compound to the subject to increase the expression or activity of a at least an active fragment of a peroxididase in the subject.

2. The method of claim 1, wherein the active fragment of the peroxidase comprises the active fragment of a peroxidase selected from the group consisting of glutathione peroxidase (Gpx) 1, Gpx2, Gpx3, Gpx4, Gpx5, Gpx6, Gpx7, Gpx8, and catalase.

3. The method of claim 1, further comprising administration of a compound to the eye of the subject to increase the expression or activity of at least an active fragment of an active oxygen species metabolizing enzyme.

4. The method of claim 3, wherein the an active oxygen species metabolizing enzyme fragment of an active oxygen species metabolizing enzyme comprises an active oxygen species metabolizing enzyme selected from the group consisting of superoxide dismutase (SOD) 1, SOD 2, and SOD3.

5. The method of claim 1, wherein the subject comprises an eye, and the disease or condition associated with oxidative stress comprises an ocular disease and the compound of claim 1 or claim 3 or both are administered to the eye.

6. The method of claim 1, wherein a compound that increases the expression or activity of the peroxide metabolizing enzyme comprises an expression construct for expression of the at least the active fragment of a peroxide metabolizing enzyme operably linked to a promoter sequence.

7. The method of claim 1, wherein a compound that increases the expression or activity of the active fragment of an active oxygen species metabolizing enzyme comprises an expression construct for expression of the at least the active fragment of the active oxygen species metabolizing enzyme operably linked to a promoter sequence.

8. The method of claim 1, wherein an active fragment of the peroxidase and an active fragment of the active oxygen species metabolizing enzyme are targeted to a single cellular compartment.

9-26. (canceled)

27. The method of claim 1, further comprising identifying a subject prone to or suffering from a disease or condition associated with oxidative stress.

28. The method of claim 27, wherein a disease or condition associated with oxidative stress is selected from the group consisting of atherosclerosis, Parkinson's disease, heart failure, myocardial infarction, Alzheimer's disease, diabetes, chronic lung disease, diseases associated with mitochondrial dysfunction, diseases associated with chronic inflammation, retinitis pigmentosa, wet age related macular degeneration, dry age related macular degeneration, diabetic retinopathy, Lebers optic neuropathy, and optic neuritis.

29. The method of claim 27, wherein a disease or condition associated with oxidative stress comprises an ocular disease or condition associated with oxidative stress.

30. (canceled)

31. The method of claim 1, wherein the disease or condition associated with oxidative stress in an eye is selected from the group consisting of atherosclerosis, Parkinson's disease, heart failure, myocardial infarction, Alzheimer's disease, diabetes, chronic lung disease, diseases associated with mitochondrial dysfunction, diseases associated with chronic inflammation, retinitis pigmentosa, wet age related macular degeneration, dry age related macular degeneration, diabetic retinopathy, Lebers optic neuropathy, and optic neuritis.

32-33. (canceled)

34. A composition comprising a compound to increase the expression or activity of a at least an active peroxide metabolizing fragment of a peroxide metabolizing enzyme in a cell.

35. The composition of claim 34, wherein the active fragment of the peroxide metabolizing enzyme comprises the active fragment of an enzyme selected from the group consisting of glutathione peroxidase (Gpx) 1, Gpx2, Gpx3, Gpx4, Gpx5, Gpx6, Gpx7, Gpx8, and catalase.

36. The composition of claim 34, further comprising a compound to increase the expression or activity of at least an active fragment of an active oxygen species metabolizing enzyme.

37. The composition of claim 36, wherein the an active oxygen species metabolizing fragment of an active oxygen species metabolizing enzyme comprises an active oxygen species metabolizing enzyme selected from the group consisting of superoxide dismutase (SOD) 1, SOD 2, and SOD3.

38. The composition of claim 34, wherein the cell is in an eye.

39-56. (canceled)


SEQUENCE LISTING (60 sequences)

SEQ ID NO: 1 - protein, 29 aa, Homo sapiens
MMSFVSLLLVGILFWATEAEQLTKCEVFQ

SEQ ID NO: 2 - protein, 4 aa, Homo sapiens
KDEL

SEQ ID NO: 3 - protein, 25 aa, Homo sapiens
MLSLRQSIRFFKPATRTLCSSRYLL

SEQ ID NO: 4 - protein, 35 aa, Homo sapiens
MLFNLRILLNNAAFRNGHNFMVRNFRCGQPLQLGS

SEQ ID NO: 5 - protein, 14 aa, Homo sapiens
MVLPRLYTATSRAA

SEQ ID NO: 6 - protein, 14 aa, Homo sapiens
  VARIANT (2)..(2): /replace=“Leu” or “Ala”
MVLPRLYTATSRAA

SEQ ID NO: 7 - protein, 8 aa, Homo sapiens
PPKKKRKV

SEQ ID NO: 8 - protein, 8 aa, Homo sapiens
GSSKSKPK

SEQ ID NO: 9 - DNA, 28 bp, Artificial Sequence
  source: /note=“Description of Artificial Sequence Synthetic primer”
gcctcgagatgtgtgctgctcggctctc

SEQ ID NO: 10 - DNA, 28 bp, Artificial Sequence
  source: /note=“Description of Artificial Sequence Synthetic primer”
gcggatccttaggagttgccagactgct

SEQ ID NO: 11 - DNA, 28 bp, Artificial Sequence
  source: /note=“Description of Artificial Sequence Synthetic primer”
gcctcgagatgtgtgcatcccgcgatga

SEQ ID NO: 12 - DNA, 28 bp, Artificial Sequence
  source: /note=“Description of Artificial Sequence Synthetic primer”
gcggatccctagagatagcacggcaggt

SEQ ID NO: 13 - DNA, 21 bp, Artificial Sequence
  source: /note=“Description of Artificial Sequence Synthetic primer”
atggcgatgaaagcggtgtgc

SEQ ID NO: 14 - DNA, 21 bp, Artificial Sequence
  source: /note=“Description of Artificial Sequence Synthetic primer”
ttactgcgcaatcccaatcac

SEQ ID NO: 15 - DNA, 21 bp, Artificial Sequence
  source: /note=“Description of Artificial Sequence Synthetic primer”
atgttgtgtcgggcggcgtgc

SEQ ID NO: 16 - DNA, 21 bp, Artificial Sequence
  source: /note=“Description of Artificial Sequence Synthetic primer”
tcacttcttgcaagctgtgta

SEQ ID NO: 17 - DNA, 18 bp, Artificial Sequence
  source: /note=“Description of Artificial Sequence Synthetic primer”
cacgctgttttgacctcc

SEQ ID NO: 18 - DNA, 16 bp, Artificial Sequence
  source: /note=“Description of Artificial Sequence Synthetic primer”
gtctggcaactcctaa

SEQ ID NO: 19 - DNA, 21 bp, Artificial Sequence
  source: /note=“Description of Artificial Sequence Synthetic primer”
catcagccctaatccatctga

SEQ ID NO: 20 - DNA, 21 bp, Artificial Sequence
  source: /note=“Description of Artificial Sequence Synthetic primer”
cgcgactaacaatcaaagtga

SEQ ID NO: 21 - DNA, 20 bp, Artificial Sequence
  source: /note=“Description of Artificial Sequence Synthetic primer”
gcttgatagcctccagcaac

SEQ ID NO: 22 - DNA, 20 bp, Artificial Sequence
  source: /note=“Description of Artificial Sequence Synthetic primer”
tctggagaagtgcggagatt

SEQ ID NO: 23 - DNA, 20 bp, Artificial Sequence
  source: /note=“Description of Artificial Sequence Synthetic primer”
agtcagggtggacctcagtg

SEQ ID NO: 24 - DNA, 29 bp, Artificial Sequence
  source: /note=“Description of Artificial Sequence Synthetic primer”
gtttaccgatgcccttggaattgacgagt

SEQ ID NO: 25 - DNA, 30 bp, Artificial Sequence
  source: /note=“Description of Artificial Sequence Synthetic primer”
gatgtggcgagatgctcttgaagtctggta

SEQ ID NO: 26 - DNA, 24 bp, Artificial Sequence
  source: /note=“Description of Artificial Sequence Synthetic primer”
catcccacctgagctcacagaaag

SEQ ID NO: 27 - DNA, 24 bp, Artificial Sequence
  source: /note=“Description of Artificial Sequence Synthetic primer”
gcctacaacagaggagcttctagc

SEQ ID NO: 28 - DNA, 24 bp, Artificial Sequence
  source: /note=“Description of Artificial Sequence Synthetic primer”
ctttctattctctgtcagcaaagc

SEQ ID NO: 29 - DNA, 24 bp, Artificial Sequence
  source: /note=“Description of Artificial Sequence Synthetic primer”
catgagtagggtaaacatggtctg

SEQ ID NO: 30 - protein, 154 aa, Homo sapiens [sequence omitted]

SEQ ID NO: 31 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 32 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 33 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 34 - protein, 222 aa, Homo sapiens; identical to SEQ ID NO: 32

SEQ ID NO: 35 - DNA, 1035 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 36 - protein, 183 aa, Homo sapiens
MLSRAVCGTSRQLAPVLGYLGSRQKHSLPDLPYDYGALEPHINAQIMQLHHSKHHAAYVNNLNVTEEKYQEALAKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 37 - DNA, 918 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 38 - protein, 240 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 39 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 40 - DNA, 2305 bp, Homo sapiens
ggcaacaggcagatttgcctgctgagggtggagacccacgagccgaggcctcctgcagtgttctgcacagcaaaccgcacgctatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgacggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacgtaagaacaaaaaaaaaaaa

SEQ ID NO: 41 - protein, 203 aa, Homo sapiens
  MOD_RES (49)..(49): Selenocysteine
MCAARLAAAAAAAQSVYAFSARPLAGGEPVSLGSLRGKVLLIENVASLCGTTVRDYTQMNELQRRLGPRGLVVLGFPCNQFGHQENAKNEEILNSLKYVRPGGGFEPNFMLFEKCEVNGAGAHPLFAFLREALPAPSDDATALMTDPKLITWSPVCRNDVAWNFEKFLVGPDGVPLRRYSRRFQTIDIEPDIEALLSQGPSCA

SEQ ID NO: 42 - DNA, 921 bp, Homo sapiens
cagttaaaaggaggcgcctgctggcctccccttacagtgcttgttcggggcgctccgctggcttcttggacaattgcgccatgtgtgctgctcggctagcggcggcggcggcggcggcccagtcggtgtatgccttctcggcgcgcccgctggccggcggggagcctgtgagcctgggctccctgcggggcaaggtactacttatcgagaatgtggcgtccctctgaggcaccacggtccgggactacacccagatgaacgagctgcagcggcgcctcggaccccggggcctggtggtgctcggcttcccgtgcaaccagtttgggcatcaggagaacgccaagaacgaagagattctgaattccctcaagtacgtccggcctggtggtgggttcgagcccaacttcatgctcttcgagaagtgcgaggtgaacggtgcgggggcgcaccctctcttcgccttcctgcgggaggccctgccagctcccagcgacgacgccaccgcgcttatgaccgaccccaagctcatcacctggtctccggtgtgtcgcaacgatgttgcctggaactttgagaagttcctggtgggccctgacggtgtgcccctacgcaggtacagccgccgcttccagaccattgacatcgagcctgacatcgaagccctgctgtctcaagggcccagctgtgcctagggcgcccctcctaccccggctgcttggcagttgcagtgctgctgtctcgggggggttttcatctatgagggtgtttcctctaaacctacgagggaggaacacctgatcttacagaaaataccacctcgagatgggtgctggtcctgttgatcccagtctctgccagaccaaggcgagtttccccactaataaagtgccgggtgtcagcagaaaaaaaaaaaaaaaaaaa

SEQ ID NO: 43 - protein, 98 aa, Homo sapiens
  MOD_RES (49)..(49): Selenocysteine
MCAARLAAAAAAAQSVYAFSARPLAGGEPVSLGSLRGKVLLIENVASLCGTTVRDYTQMNELQRRLGPRGLVVLGFPCNQFGHQVRRAERGGAGADVQ

SEQ ID NO: 44 - DNA, 1200 bp, Homo sapiens
cagttaaaaggaggcgcctgctggcctccccttacagtgcttgttcggggcgctccgctggcttcttggacaattgcgccatgtgtgctgctcggctagcggcggcggcggcggcggcccagtcggtgtatgccttctcggcgcgcccgctggccggcggggagcctgtgagcctgggctccctgcggggcaaggtactacttatcgagaatgtggcgtccctctgaggcaccacggtccgggactacacccagatgaacgagctgcagcggcgcctcggaccccggggcctggtggtgctcggcttcccgtgcaaccagtttgggcatcaggtgcgccgggcggagcggggcggggcgggggcggacgtgcagtagtggctgggggcgccggcggtgtgctggtgggtgccgtcggctccatgcgcggagagtctggctactctctcgtttcctttctgttgctcgtagctgctgaaattcctctccgcccttgggattgcgcatggagggcaaaatcccggtgactcatagaaaatctcccttgtttgtggttagaacgtttctctcctcctcttgaccccgggttctagctgcccttctctcctgtaggagaacgccaagaacgaagagattctgaattccctcaagtacgtccggcctggtggtgggttcgagcccaacttcatgctcttcgagaagtgcgaggtgaacggtgcgggggcgcaccctctcttcgccttcctgcgggaggccctgccagctcccagcgacgacgccaccgcgcttatgaccgaccccaagctcatcacctggtctccggtgtgtcgcaacgatgttgcctggaactttgagaagttcctggtgggccctgacggtgtgcccctacgcaggtacagccgccgcttccagaccattgacatcgagcctgacatcgaagccctgctgtctcaagggcccagctgtgcctagggcgcccctcctaccccggctgcttggcagttgcagtgctgctgtctcgggggggttttcatctatgagggtgtttcctctaaacctacgagggaggaacacctgatcttacagaaaataccacctcgagatgggtgctggtcctgttgatcccagtctctgccagaccaaggcgagtttccccactaataaagtgccgggtgtcagcagaaaaaaaaaaaaaaaaaaa

SEQ ID NO: 45 - protein, 197 aa, Homo sapiens
  MOD_RES (73)..(73): Selenocysteine
MSLGRLCRLLKPALLCGALAAPGLAGTMCASRDDWRCARSMHEFSAKDIDGHMVNLDKYRGFVCIVTNVASQCGKTEVNYTQLVDLHARYAECGLRILAFPCNQFGKQEPGSNEEIKEFAAGYNVKFDMFSKICVNGDDAHPLWKWMKIQPKGKGILGNAIKWNFTKFLIDKNGCVVKRYGPMEEPLVIEKDLPHYF

SEQ ID NO: 46 - DNA, 942 bp, Homo sapiens
gagcgctctggagggcgtggccgtgggaaaggaggcgcggaaagccgacgcgcgtccattggtcggctggacgaggggaggagccgctggctcccagccccgccgcgatgagcctcggccgcctttgccgcctactgaagccggcgctgctctgtggggctctggccgcgcctggcctggccgggaccatgtgcgcgtcccgggacgactggcgctgtgcgcgctccatgcacgagttttccgccaaggacatcgacgggcacatggttaacctggacaagtaccggggcttcgtgtgcatcgtcaccaacgtggcctcccagtgaggcaagaccgaagtaaactacactcagctcgtcgacctgcacgcccgatacgctgagtgtggtttgcggatcctggccttcccgtgtaaccagttcgggaagcaggagccagggagtaacgaagagatcaaagagttcgccgcgggctacaacgtcaaattcgatatgttcagcaagatctgcgtgaacggggacgacgcccacccgctgtggaagtggatgaagatccaacccaagggcaagggcatcctgggaaatgccatcaagtggaacttcaccaagttcctcatcgacaagaacggctgcgtggtgaagcgctacggacccatggaggagcccctggtgatagagaaggacctgccccactatttctagctccacaagtgtgtggccccgcccgagcccctgcccacgcccttggagccttccaccggcactcatgacggcctgcctgcaaacctgctggtggggcagacccgaaaatccagcgtgcaccccgccggaggaaggtcccatggcctgctgggcttggctcggcgcccccacccctggctaccttgtgggaataaacagacaaattagcctgctggaaaaaaaaaaaaaaaaaaaaaaaaaa

SEQ ID NO: 47 - protein, 190 aa, Homo sapiens
  MOD_RES (40)..(40): Selenocysteine
MAFIAKSFYDLSAISLDGEKVDFNTFRGRAVLIENVASLCGTTTRDFTQLNELQCRFPRRLVVLGFPCNQFGHQENCQNEEILNSLKYVRPGGGYQPTFTLVQKCEVNGQNEHPVFAYLKDKLPYPYDDPFSLMTDPKLIIWSPVRRSDVAWNFEKFLIGPEGEPFRRYSRTFPTINIEPDIKRLLKVAI

SEQ ID NO: 48 - protein, 226 aa, Homo sapiens
  MOD_RES (73)..(73): Selenocysteine
MARLLQASCLLSLLLAGFVSQSRGQEKSKMDCHGGISGTIYEYGALTIDGEEYIPFKQYAGKYVLFVNVASYCGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 49 - protein, 221 aa, Homo sapiens
MTTQLRVVHLLPLLLACFVQTSPKQEKMKMDCHKDEKGTIYDYEAIALNKNEYVSFKQYVGKHILFVNVATYCGLTAQYPELNALQEELKPYGLVVLGFPCNQFGKQEPGDNKEILPGLKYVRPGGGFVPSFQLFEKGDVNGEKEQKVFSFLKHSCPHPSEILGTFKSISWDPVKVHDIRWNFEKFLVGPDGIPVMRWSHRATVSSVKTDILAYLKQFKTK

SEQ ID NO: 50 - protein, 221 aa, Homo sapiens
  MOD_RES (73)..(73): Selenocysteine
MFQQFQASCLVLLFLVGFAQQTLKPQNRKVDCNKGVTGTIYEYGALTLNGEEYIQFKQFAGKHVLFVNVAAYCGLAAQYPELNALQEELKNFGVIVLAFPCNQFGKQEPGTNSEILLGLKYVCPGSGFVPSFQLFEKGDVNGEKEQKVFTFLKNSCPPTSDLLGSSSQLFWEPMKVHDIRWNFEKFLVGPDGVPVMHWFHQAPVSTVKSDILEYLKQFNTH

SEQ ID NO: 51 - protein, 187 aa, Homo sapiens
MVAATVAAAWLLLWAAACAQQEQDFYDFKAVNIRGKLVSLEKYRGSVSLVVNVASECGFTDQHYRALQQLQRDLGPHHFNVLAFPCNQFGQQEPDSNKEIESFARRTYSVSFPMFSKIAVTGTGAHPAFKYLAQTSGKEPTWNFWKYLVAPDGKVVGAWDPTVSVEEVRPQITALVRKLILLKREDL

SEQ ID NO: 52 - protein, 209 aa, Homo sapiens
MEPLAAYPLKCSGPRAKVFAVLLSIVLCTVTLFLLQLKFLKPKINSFYAFEVKDAKGRTVSLEKYKGKVSLVVNVASDCQLTDRNYLGLKELHKEFGPSHFSVLAFPCNQFGESEPRPSKEVESFARKNYGVTFPIFHKIKILGSEGEPAFRFLVDSSKKEPRWNFWKYLVNPEGQVVKFWKPEEPIEVIRPDIAALVRQVIIKKKEDL

SEQ ID NO: 53 - DNA, 1741 bp, Artificial Sequence
  source: /note=“Description of Artificial Sequence Synthetic polynucleotide”
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcttgatcgaagcttgcccacc

SEQ ID NO: 54 - DNA, 953 bp, Artificial Sequence
  source: /note=“Description of Artificial Sequence Synthetic polynucleotide”
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 55 - protein, 460 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 56 - protein, 460 aa, Homo sapiens; identical to SEQ ID NO: 55

SEQ ID NO: 57 - protein, 465 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 58 - protein, 133 aa, Homo sapiens
MGCRGCLPGAAPHRVRLPAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 59 - protein, 185 aa, Homo sapiens
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 60 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI